SEQUENCE LISTING (168 sequences)

SEQ ID NO: 1 - protein, 11 aa, synthetic construct
RASQDISNYLN

SEQ ID NO: 2 - protein, 7 aa, synthetic construct
YTSILHS

SEQ ID NO: 3 - protein, 9 aa, synthetic construct
QQGNTLPWT

SEQ ID NO: 4 - protein, 5 aa, synthetic construct
IYDMS

SEQ ID NO: 5 - protein, 17 aa, synthetic construct
YISSGGGTTYYPDTVKG

SEQ ID NO: 6 - protein, 14 aa, synthetic construct
HSGYGSSYGVLFAY

SEQ ID NO: 7 - protein, 108 aa, synthetic construct
DIQLTQTTSSLSASLGDRVTISCRASQDISNYLNWYQQKPDGTVKLLIYYTSILHSGVPSRFSGSGSGTDYSLTISNLEQEDFATYFCQQGNTLPWTFGGGTKLEIKR

SEQ ID NO: 8 - protein, 123 aa, synthetic construct
QVQLQESGGGLVKPGGSLKLSCAASGFAFSIYDMSWVRQTPEKRLEWVAYISSGGGTTYYPDTVKGRFTISRDNAKNTLYLQMSSLKSEDTAMYYCARHSGYGSSYGVLFAYWGQGTLVTVSS

SEQ ID NO: 9 - protein, 108 aa, synthetic construct
DIQLTQSPSSLSASVGDRVTISCRASQDISNYLNWYQQKPGKAPKLLIYYTSILHSGVPSRFSGSGSGTEFTLTISSLQPEDFATYFCQQGNTLPWTFGGGTKVEIKR

SEQ ID NO: 10 - protein, 123 aa, synthetic construct
EVQLVESGGGLVQPGGSLRLSCAASGFSFSIYDMSWVRQAPGKGLEWVAYISSGGGTTYYPDTVKGRFTISRDNAKNSLYLQMNSLRVEDTALYYCARHSGYGSSYGVLFAYWGQGTLVTVSS

SEQ ID NO: 11 - protein, 17 aa, synthetic construct
KSSQSVLYSANHKNYLA

SEQ ID NO: 12 - protein, 7 aa, synthetic construct
WASTRES

SEQ ID NO: 13 - protein, 8 aa, synthetic construct
HQYLSSWT

SEQ ID NO: 14 - protein, 5 aa, synthetic construct
SYWLH

SEQ ID NO: 15 - protein, 17 aa, synthetic construct
YINPRNDYTEYNQNFKD

SEQ ID NO: 16 - protein, 7 aa, synthetic construct
RDITTFY

SEQ ID NO: 17 - protein, 111 aa, synthetic construct
DIQLTQSPSSLAVSAGENVTMSCKSSQSVLYSANHKNYLAWYQQKPGQSPKLLIYWASTRESGVPDRFTGSGSGTDFTLTISRVQVEDLAIYYCHQYLSSWTGGGTKLEIK

SEQ ID NO: 18 - protein, 116 aa, synthetic construct
QVQLQESGAELSKPGASVKMSCKASGYTFTSYWLHWIKQRPGQGLEWIGYINPRNDYTEYNQNFKDKATLTADKSSSTAYMQLSSLTSEDSAVYYCARRDITTFYWGQGTTLTVSS

SEQ ID NO: 19 - protein, 112 aa, synthetic construct [sequence omitted]

SEQ ID NO: 20 - protein, 116 aa, synthetic construct [sequence omitted]

SEQ ID NO: 21 - protein, 15 aa, synthetic construct
RASESLDNYGIRFLT

SEQ ID NO: 22 - protein, 7 aa, synthetic construct
AASNQGS

SEQ ID NO: 23 - protein, 9 aa, synthetic construct
QQTKEVPWS

SEQ ID NO: 24 - protein, 5 aa, synthetic construct
DSNIH

SEQ ID NO: 25 - protein, 17 aa, synthetic construct
YIYPYNGGTDYNQKFKN

SEQ ID NO: 26 - protein, 7 aa, synthetic construct
GNPWLAY

SEQ ID NO: 27 - protein, 112 aa, synthetic construct
DIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKR

SEQ ID NO: 28 - protein, 116 aa, synthetic construct [sequence omitted]

SEQ ID NO: 29 - protein, 17 aa, synthetic construct
KSSQSVFFSSSQKNYLA

SEQ ID NO: 30 - protein, 7 aa, synthetic construct
WASTRES

SEQ ID NO: 31 - protein, 8 aa, synthetic construct
HQYLSSRT

SEQ ID NO: 32 - protein, 5 aa, synthetic construct
SYYIH

SEQ ID NO: 33 - protein, 17 aa, synthetic construct
VIYPGNDDISYNQKFQG

SEQ ID NO: 34 - protein, 9 aa, synthetic construct
EVRLRYFDV

SEQ ID NO: 35 - protein, 113 aa, synthetic construct
EIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKR

SEQ ID NO: 36 - protein, 119 aa, synthetic construct
QVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTTVSS

SEQ ID NO: 37 - protein, 15 aa, synthetic construct
RASESVDNYGISFMN

SEQ ID NO: 38 - protein, 7 aa, synthetic construct
AASNQGS

SEQ ID NO: 39 - protein, 9 aa, synthetic construct
QQSKEVPWT

SEQ ID NO: 40 - protein, 5 aa, synthetic construct
DYNMH

SEQ ID NO: 41 - protein, 17 aa, synthetic construct
YIYPYNGGTGYNQKFKS

SEQ ID NO: 42 - protein, 7 aa, synthetic construct
GRPAMDY

SEQ ID NO: 43 - protein, 111 aa, synthetic construct
DIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIK

SEQ ID NO: 44 - protein, 116 aa, synthetic construct
QVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSS

SEQ ID NO: 45 - protein, 16 aa, synthetic construct
RSSQSLVHRNGNTYLH

SEQ ID NO: 46 - protein, 7 aa, synthetic construct
TVSNRFS

SEQ ID NO: 47 - protein, 9 aa, synthetic construct
SQSSHVPPT

SEQ ID NO: 48 - protein, 5 aa, synthetic construct
NYGVN

SEQ ID NO: 49 - protein, 17 aa, synthetic construct
WINPNTGEPTFDDDFKG

SEQ ID NO: 50 - protein, 11 aa, synthetic construct
SRGKNEAWFAY

SEQ ID NO: 51 - protein, 113 aa, synthetic construct
DIQLTQTPLSLPVSLGDQASISCRSSQSLVHRNGNTYLHWYLQKPGQSPKLLIYTVSNRFSGVPDRFSGSGSGTDFTLKISRVEAEDLGLYFCSQSSHVPPTFGAGTKLEIKR

SEQ ID NO: 52 - protein, 120 aa, synthetic construct
QVQLQQSGPELKKPGETVKVTCKTSGYTFTNYGVNWIKQTPGEGLQWMGWINPNTGEPTFDDDFKGRFAFSLESSASTAFLQISNLKNEDMGTYFCSRSRGKNEAWFAYWGQGTLVTVSS

SEQ ID NO: 53 - protein, 113 aa, synthetic construct
DIQLTQSPLSLPVTLGQPASISCRSSQSLVHRNGNTYLHWFQQRPGQSPRLLIYTVSNRFSGVPDRFSGSGSGTDFTLKISRVEAEDVGVYFCSQSSHVPPTFGAGTRLEIKR

SEQ ID NO: 54 - protein, 120 aa, synthetic construct
QVQLQQSGSELKKPGASVKVSCKASGYTFTNYGVNWIKQAPGQGLQWMGWINPNTGEPTFDDDFKGRFAFSLDTSVSTAYLQISSLKADDTAVYFCSRSRGKNEAWFAYWGQGTLVTVSS

SEQ ID NO: 55 - protein, 11 aa, synthetic construct
RASQSISSYLN

SEQ ID NO: 56 - protein, 7 aa, synthetic construct
AASSLQS

SEQ ID NO: 57 - protein, 9 aa, synthetic construct
QQSYSTPLT

SEQ ID NO: 58 - protein, 5 aa, synthetic construct
SYGMH

SEQ ID NO: 59 - protein, 17 aa, synthetic construct
VIWFDGTKKYYTDSVKG

SEQ ID NO: 60 - protein, 15 aa, synthetic construct
DRGIGARRGPYYMDV

SEQ ID NO: 61 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKR

SEQ ID NO: 62 - protein, 124 aa, synthetic construct
QVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSS

SEQ ID NO: 63 - protein, 124 aa, synthetic construct
EVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSS

SEQ ID NO: 64 - protein, 16 aa, synthetic construct
RSSQSIVHSNGNTYLE

SEQ ID NO: 65 - protein, 7 aa, synthetic construct
KVSNRFS

SEQ ID NO: 66 - protein, 9 aa, synthetic construct
FQGSHVPPT

SEQ ID NO: 67 - protein, 5 aa, synthetic construct
SFGMH

SEQ ID NO: 68 - protein, 17 aa, synthetic construct
YISSGSSTIYYGDTVKG

SEQ ID NO: 69 - protein, 15 aa, synthetic construct
EGGYYYGRSYYTMDY

SEQ ID NO: 70 - protein, 112 aa, synthetic construct
DVVMTQSPLSLPVTPGAPASISCRSSQSIVHSNGNTYLEWYLQKPGQSPKLLIYKVSNRFSGVPDRFSGSGSGTDFTLRISRVEAEDVGIYYCFQGSHVPPTFGPGTKLEIK

SEQ ID NO: 71 - protein, 123 aa, synthetic construct
EVQLVESGGGLVQPGGSLRLSCSASGFTFSSFGMHWVRQAPGKGLEWVAYISSGSSTIYYGDTVKGRFTISRDNAKNSLFLQMSSLRAEDTAVYYCAREGGYYYGRSYYTMDYWGQGTTVTVS

SEQ ID NO: 72 - protein, 12 aa, synthetic construct
RASQSVSSSYLA

SEQ ID NO: 73 - protein, 7 aa, synthetic construct
GASSRAT

SEQ ID NO: 74 - protein, 9 aa, synthetic construct
LQIYNMPIT

SEQ ID NO: 75 - protein, 5 aa, synthetic construct
SYAMS

SEQ ID NO: 76 - protein, 16 aa, synthetic construct
INASGTRTYYADSVKG

SEQ ID NO: 77 - protein, 17 aa, synthetic construct
GKGNTHKPYGYVRYFDV

SEQ ID NO: 78 - protein, 110 aa, synthetic construct
DIVLTQSPATLSLSPGERATLSCRASQSVSSSYLAWYQQKPGQAPRLLIYGASSRATGVPARFSGSGSGTDFTLTISSLEPEDFATYYCLQIYNMPITFGQGTKVEIKRT

SEQ ID NO: 79 - protein, 124 aa, synthetic construct
QVQLVESGGGLVQPGGSLRLSCAASGFTFSSYAMSWVRQAPGKGLEWVAINASGTRTYYADSVKGRFTISRDNSKNTLYLQMNLRAEDTAVYYCARGKGNTHKPYGYVRYFDVWGQGTLVTVSS

SEQ ID NO: 80 - protein, 16 aa, synthetic construct
RSSQSLVYSNGDTYLH

SEQ ID NO: 81 - protein, 6 aa, synthetic construct
KVSNRF

SEQ ID NO: 82 - protein, 9 aa, synthetic construct
SQSTHVPWT

SEQ ID NO: 83 - protein, 10 aa, synthetic construct
GFTFSSYGMS

SEQ ID NO: 84 - protein, 17 aa, synthetic construct
SINSNGGSTYYPDSVKG

SEQ ID NO: 85 - skipped

SEQ ID NO: 86 - protein, 113 aa, synthetic construct
DIVMTQSPLSLPVTPGEPASISCRSSQSLVYSNGDTYLHWYLQKPGQSPQLLIYKVSNRFSGVPDRFSGSGSGTDFTLKISRVEAEDVGVYYCSQSTHVPWTFGQGTKVEIKR

SEQ ID NO: 87 - protein, 112 aa, synthetic construct
EVQLVESGGGLVQPGGSLRLSCAASGFTFSSYGMSWVRQAPGKGLELVASINSNGGSTYYPDSVKGRFTISRDNAKNSLYLQMNSLRAEDTAVYYCASGDYWGQGTTVTVSS

SEQ ID NO: 88 - protein, 11 aa, synthetic construct
RASQDISTSLA

SEQ ID NO: 89 - protein, 7 aa, synthetic construct
GTSYRAT

SEQ ID NO: 90 - protein, 11 aa, synthetic construct
QHRRDWPPGAT

SEQ ID NO: 91 - protein, 5 aa, synthetic construct
DYWMH

SEQ ID NO: 92 - protein, 17 aa, synthetic construct
RLKSDGSSRSYADFVKG

SEQ ID NO: 93 - protein, 9 aa, synthetic construct
ELRGWSIEY

SEQ ID NO: 94 - protein, 109 aa, synthetic construct
ELVLTQSPATLSLSPGERATLFCRASQDISTSLAWYQQKPGQVPRLLIYGTSYRATGISARFSGRGSGTDFTLTISSLEPEDFALYYCQHRRDWPPGATFGQGTRLEIK

SEQ ID NO: 95 - protein, 118 aa, synthetic construct
EVQLLESGGGLVQPGGSLRLSCTASGFVFSDYWMHWVRQAPGKGLVWISRLKSDGSSRSYADFVKGRFTVSRDNVKNTLFLQINSLRAEDTAVYYCARELRGWSIEYWGHGTQVTVSS

SEQ ID NO: 96 - protein, 211 aa, synthetic construct
DIQLTQTTSSLSASLGDRVTISCRASQDISNYLNWYQQKPDGTVKLLIYYTSILHSGVPSRFSGSGSGTDYSLTISNLEQEDFATYFCQQGNTLPWTFGGGTKLEIKAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 97 - protein, 708 aa, synthetic construct
QVQLQESGGGLVKPGGSLKLSCAASGFAFSIYDMSWVRQTPEKRLEWVAYISSGGGTTYYPDTVKGRFTISRDNAKNTLYLQMSSLKSEDTAMYYCARHSGYGSSYGVLFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFTFSSYGMSWVRQAPGKGLELVASINSNGGSTYYPDSVKGRFTISRDNAKNSLYLQMNSLRAEDTAVYYCASGDYWGQGTTVTVSSGGGGSGGGGSGGGGSDIVMTQSPLSLPVTPGEPASISCRSSQSLVYSNGDTYLHWYLQKPGQSPQLLIYKVSNRFSGVPDRFSGSGSGTDFTLKISRVEAEDVGVYYCSQSTHVPWTFGQGTKVEIKR

SEQ ID NO: 98 - protein, 718 aa, synthetic construct
QVQLQESGGGLVKPGGSLKLSCAASGFAFSIYDMSWVRQTPEKRLEWVAYISSGGGTTYYPDTVKGRFTISRDNAKNTLYLQMSSLKSEDTAMYYCARHSGYGSSYGVLFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCSASGFTFSSFGMHWVRQAPGKGLEWVAYISSGSSTIYYGDTVKGRFTISRDNAKNSLFLQMSSLRAEDTAVYYCAREGGYYYGRSYYTMDYWGQGTTVTVSGGGGSGGGGSGGGGSDVVMTQSPLSLPVTPGAPASISCRSSQSIVHSNGNTYLEWYLQKPGQSPKLLIYKVSNRFSGVPDRFSGSGSGTDFTLRISRVEAEDVGIYYCFQGSHVPPTFGPGTKLEIK

SEQ ID NO: 99 - protein, 718 aa, synthetic construct
QVQLQESGGGLVKPGGSLKLSCAASGFAFSIYDMSWVRQTPEKRLEWVAYISSGGGTTYYPDTVKGRFTISRDNAKNTLYLQMSSLKSEDTAMYYCARHSGYGSSYGVLFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFTFSSYAMSWVRQAPGKGLELVAINASGTRTYYADSVKGRFTISRDNAKNSLYLQMNSLRAEDTAVYYCASGKGNTHKPYGYVRYFDVWGQGTTVTVSSGGGGSGGGGSGGGGSDIVLTQSPATLSLSPGERATLSCRASQSVSSSYLAWYQQKPGQAPRLLIYGASSRATGVPARFSGSGSGTDFTLTISSLEPEDFATYYCLQIYNMPITFGQGTKVEIKRT

SEQ ID NO: 100 - protein, 715 aa, synthetic construct
QVQLQESGGGLVKPGGSLKLSCAASGFAFSIYDMSWVRQTPEKRLEWVAYISSGGGTTYYPDTVKGRFTISRDNAKNTLYLQMSSLKSEDTAMYYCARHSGYGSSYGVLFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKR

SEQ ID NO: 101 - protein, 714 aa, synthetic construct
QVQLQESGGGLVKPGGSLKLSCAASGFAFSIYDMSWVRQTPEKRLEWVAYISSGGGTTYYPDTVKGRFTISRDNAKNTLYLQMSSLKSEDTAMYYCARHSGYGSSYGVLFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLLESGGGLVQPGGSLRLSCTASGFVFSDYWMHWVRQAPGKGLVWISRLKSDGSSRSYADFVKGRFTVSRDNVKNTLFLQINSLRAEDTAVYYCARELRGWSIEYWGHGTQVTVSSASTKGGGGSGGGGSGGGGSELVLTQSPATLSLSPGERATLFCRASQDISTSLAWYQQKPGQVPRLLIYGTSYRATGISARFSGRGSGTDFTLTISSLEPEDFALYYCQHRRDWPPGATFGQGTRLEIK

SEQ ID NO: 102 - protein, 214 aa, synthetic construct
DIQLTQSPSSLSASVGDRVTISCRASQDISNYLNWYQQKPGKAPKLLIYYTSILHSGVPSRFSGSGSGTEFTLTISSLQPEDFATYFCQQGNTLPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - protein, 721 aa, synthetic construct
EVQLVESGGGLVQPGGSLRLSCAASGFSFSIYDMSWVRQAPGKGLEWVAYISSGGGTTYYPDTVKGRFTISRDNAKNSLYLQMNSLRVEDTALYYCARHSGYGSSYGVLFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKPRGGGGSGGGGSGGGGSEVQLVESGGGLVQPGGSLRLSCSASGFTFSSFGMHWVRQAPGKGLEWVAYISSGSSTIYYGDTVKGRFTISRDNAKNSLFLQMSSLRAEDTAVYYCAREGGYYYGRSYYTMDYWGQGTTVTVSGGGGSGGGGSGGGGSDVVMTQSPLSLPVTPGAPASISCRSSQSIVHSNGNTYLEWYLQKPGQSPKRLLIYKVSNRFSGVPDRFSGSGSGTDFTLRISRVEAEDVGIYYCFQGSHVPPTFGPGTKLEIK

SEQ ID NO: 104 - protein, 719 aa, synthetic construct
EVQLVESGGGLVQPGGSLRLSCAASGFSFSIYDMSWVRQAPGKGLEWVAYISSGGGTTYYPDTVKGRFTISRDNAKNSLYLQMNSLRVEDTALYYCARHSGYGSSYGVLFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKPRGGGGSGGGGSGGGGSQVQLVESGGGLVQPGGSLRLSCAASGFTFSSYAMSWVRQAPGKGLEWVAINASGTRTYYADSVKGRFTISRDNSKNTLYLQMNLRAEDTAVYYCARGKGNTHKPYGYVRYFDVWGQGTLVTVSSGGGGSGGGGSGGGGSDIVLTQSPATLSLSPGERATLSCRASQSVSSSYLAWYQQKPGQAPRLLIYGASSRATGVPARFSGSGSGTDFTLTISSLEPEDFATYYCLQIYNMPITFGQGTKVEIKRT

SEQ ID NO: 105 - protein, 717 aa, synthetic construct
EVQLVESGGGLVQPGGSLRLSCAASGFSFSIYDMSWVRQAPGKGLEWVAYISSGGGTTYYPDTVKGRFTISRDNAKNSLYLQMNSLRVEDTALYYCARHSGYGSSYGVLFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKPRGGGGSGGGGSGGGGSQVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKR

SEQ ID NO: 106 - protein, 716 aa, synthetic construct
EVQLVESGGGLVQPGGSLRLSCAASGFSFSIYDMSWVRQAPGKGLEWVAYISSGGGTTYYPDTVKGRFTISRDNAKNSLYLQMNSLRVEDTALYYCARHSGYGSSYGVLFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKPRGGGGSGGGGSGGGGSEVQLLESGGGLVQPGGSLRLSCTASGFVFSDYWMHWVRQAPGKGLVWISRLKSDGSSRSYADFVKGRFTVSRDNVKNTLFLQINSLRAEDTAVYYCARELRGWSIEYWGHGTQVTVSSASTKGGGGSGGGGSGGGGSELVLTQSPATLSLSPGERATLFCRASQDISTSLAWYQQKPGQVPRLLIYGTSYRATGISARFSGRGSGTDFTLTISSLEPEDFALYYCQHRRDWPPGATFGQGTRLEIK

SEQ ID NO: 107 - protein, 710 aa, synthetic construct
EVQLVESGGGLVQPGGSLRLSCAASGFSFSIYDMSWVRQAPGKGLEWVAYISSGGGTTYYPDTVKGRFTISRDNAKNSLYLQMNSLRVEDTALYYCARHSGYGSSYGVLFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKPRGGGGSGGGGSGGGGSEVQLVESGGGLVQPGGSLRLSCAASGFTFSSYGMSWVRQAPGKGLELVASINSNGGSTYYPDSVKGRFTISRDNAKNSLYLQMNSLRAEDTAVYYCASGDYWGQGTTVTVSSGGGGSGGGGSGGGGSDIVMTQSPLSLPVTPGEPASISCRSSQSLVYSNGDTYLHWYLQKPGQSPQLLIYKVSNRFSGVPDRFSGSGSGTDFTLKISRVEAEDVGVYYCSQSTHVPWTFGQGTKVEIKR

SEQ ID NO: 108 - protein, 218 aa, synthetic construct
DIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 109 - protein, 710 aa, synthetic construct
EVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKPRGGGGSGGGGSGGGGSQVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKR

SEQ ID NO: 110 - protein, 703 aa, synthetic construct
EVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKPRGGGGSGGGGSGGGGSEVQLVESGGGLVQPGGSLRLSCAASGFTFSSYGMSWVRQAPGKGLELVASINSNGGSTYYPDSVKGRFTISRDNAKNSLYLQMNSLRAEDTAVYYCASGDYWGQGTTVTVSSGGGGSGGGGSGGGGSDIVMTQSPLSLPVTPGEPASISCRSSQSLVYSNGDTYLHWYLQKPGQSPQLLIYKVSNRFSGVPDRFSGSGSGTDFTLKISRVEAEDVGVYYCSQSTHVPWTFGQGTKVEIKR

SEQ ID NO: 111 - protein, 714 aa, synthetic construct
EVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKPRGGGGSGGGGSGGGGSEVQLVESGGGLVQPGGSLRLSCSASGFTFSSFGMHWVRQAPGKGLEWVAYISSGSSTIYYGDTVKGRFTISRDNAKNSLFLQMSSLRAEDTAVYYCAREGGYYYGRSYYTMDYWGQGTTVTVSGGGGSGGGGSGGGGSDVVMTQSPLSLPVTPGAPASISCRSSQSIVHSNGNTYLEWYLQKPGQSPKRLLIYKVSNRFSGVPDRFSGSGSGTDFTLRISRVEAEDVGIYYCFQGSHVPPTFGPGTKLEIK

SEQ ID NO: 112 - protein, 712 aa, synthetic construct
EVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKPRGGGGSGGGGSGGGGSQVQLVESGGGLVQPGGSLRLSCAASGFTFSSYAMSWVRQAPGKGLEWVAINASGTRTYYADSVKGRFTISRDNSKNTLYLQMNLRAEDTAVYYCARGKGNTHKPYGYVRYFDVWGQGTLVTVSSGGGGSGGGGSGGGGSDIVLTQSPATLSLSPGERATLSCRASQSVSSSYLAWYQQKPGQAPRLLIYGASSRATGVPARFSGSGSGTDFTLTISSLEPEDFATYYCLQIYNMPITFGQGTKVEIKRT

SEQ ID NO: 113 - protein, 709 aa, synthetic construct
EVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKPRGGGGSGGGGSGGGGSEVQLLESGGGLVQPGGSLRLSCTASGFVFSDYWMHWVRQAPGKGLVWISRLKSDGSSRSYADFVKGRFTVSRDNVKNTLFLQINSLRAEDTAVYYCARELRGWSIEYWGHGTQVTVSSASTKGGGGSGGGGSGGGGSELVLTQSPATLSLSPGERATLFCRASQDISTSLAWYQQKPGQVPRLLIYGTSYRATGISARFSGRGSGTDFTLTISSLEPEDFALYYCQHRRDWPPGATFGQGTRLEIK

SEQ ID NO: 114 - protein, 220 aa, synthetic construct
EIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - protein, 713 aa, synthetic construct
QVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKPRGGGGSGGGGSGGGGSQVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKR

SEQ ID NO: 116 - protein, 706 aa, synthetic construct
QVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKPRGGGGSGGGGSGGGGSEVQLVESGGGLVQPGGSLRLSCAASGFTFSSYGMSWVRQAPGKGLELVASINSNGGSTYYPDSVKGRFTISRDNAKNSLYLQMNSLRAEDTAVYYCASGDYWGQGTTVTVSSGGGGSGGGGSGGGGSDIVMTQSPLSLPVTPGEPASISCRSSQSLVYSNGDTYLHWYLQKPGQSPQLLIYKVSNRFSGVPDRFSGSGSGTDFTLKISRVEAEDVGVYYCSQSTHVPWTFGQGTKVEIKR

SEQ ID NO: 117 - protein, 717 aa, synthetic construct
QVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKPRGGGGSGGGGSGGGGSEVQLVESGGGLVQPGGSLRLSCSASGFTFSSFGMHWVRQAPGKGLEWVAYISSGSSTIYYGDTVKGRFTISRDNAKNSLFLQMSSLRAEDTAVYYCAREGGYYYGRSYYTMDYWGQGTTVTVSGGGGSGGGGSGGGGSDVVMTQSPLSLPVTPGAPASISCRSSQSIVHSNGNTYLEWYLQKPGQSPKRLLIYKVSNRFSGVPDRFSGSGSGTDFTLRISRVEAEDVGIYYCFQGSHVPPTFGPGTKLEIK

SEQ ID NO: 118 - protein, 715 aa, synthetic construct
QVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKPRGGGGSGGGGSGGGGSQVQLVESGGGLVQPGGSLRLSCAASGFTFSSYAMSWVRQAPGKGLEWVAINASGTRTYYADSVKGRFTISRDNSKNTLYLQMNLRAEDTAVYYCARGKGNTHKPYGYVRYFDVWGQGTLVTVSSGGGGSGGGGSGGGGSDIVLTQSPATLSLSPGERATLSCRASQSVSSSYLAWYQQKPGQAPRLLIYGASSRATGVPARFSGSGSGTDFTLTISSLEPEDFATYYCLQIYNMPITFGQGTKVEIKRT

SEQ ID NO: 119 - protein, 712 aa, synthetic construct
QVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKPRGGGGSGGGGSGGGGSEVQLLESGGGLVQPGGSLRLSCTASGFVFSDYWMHWVRQAPGKGLVWISRLKSDGSSRSYADFVKGRFTVSRDNVKNTLFLQINSLRAEDTAVYYCARELRGWSIEYWGHGTQVTVSSASTKGGGGSGGGGSGGGGSELVLTQSPATLSLSPGERATLFCRASQDISTSLAWYQQKPGQVPRLLIYGTSYRATGISARFSGRGSGTDFTLTISSLEPEDFALYYCQHRRDWPPGATFGQGTRLEIK

SEQ ID NO: 120 - protein, 218 aa, synthetic construct
DIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 121 - protein, 706 aa, synthetic construct
QVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGPRGGGGSGGGGSGGGGSQVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKR

SEQ ID NO: 122 - protein, 699 aa, synthetic construct
QVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGPRGGGGSGGGGSGGGGSEVQLVESGGGLVQPGGSLRLSCAASGFTFSSYGMSWVRQAPGKGLELVASINSNGGSTYYPDSVKGRFTISRDNAKNSLYLQMNSLRAEDTAVYYCASGDYWGQGTTVTVSSGGGGSGGGGSGGGGSDIVMTQSPLSLPVTPGEPASISCRSSQSLVYSNGDTYLHWYLQKPGQSPQLLIYKVSNRFSGVPDRFSGSGSGTDFTLKISRVEAEDVGVYYCSQSTHVPWTFGQGTKVEIKR

SEQ ID NO: 123 - protein, 710 aa, synthetic construct
QVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGPRGGGGSGGGGSGGGGSEVQLVESGGGLVQPGGSLRLSCSASGFTFSSFGMHWVRQAPGKGLEWVAYISSGSSTIYYGDTVKGRFTISRDNAKNSLFLQMSSLRAEDTAVYYCAREGGYYYGRSYYTMDYWGQGTTVTVSGGGGSGGGGSGGGGSDVVMTQSPLSLPVTPGAPASISCRSSQSIVHSNGNTYLEWYLQKPGQSPKRLLIYKVSNRFSGVPDRFSGSGSGTDFTLRISRVEAEDVGIYYCFQGSHVPPTFGPGTKLEIK

SEQ ID NO: 124 - protein, 708 aa, synthetic construct
QVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGPRGGGGSGGGGSGGGGSQVQLVESGGGLVQPGGSLRLSCAASGFTFSSYAMSWVRQAPGKGLEWVAINASGTRTYYADSVKGRFTISRDNSKNTLYLQMNLRAEDTAVYYCARGKGNTHKPYGYVRYFDVWGQGTLVTVSSGGGGSGGGGSGGGGSDIVLTQSPATLSLSPGERATLSCRASQSVSSSYLAWYQQKPGQAPRLLIYGASSRATGVPARFSGSGSGTDFTLTISSLEPEDFATYYCLQIYNMPITFGQGTKVEIKRT

SEQ ID NO: 125 - protein, 705 aa, synthetic construct
QVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGPRGGGGSGGGGSGGGGSEVQLLESGGGLVQPGGSLRLSCTASGFVFSDYWMHWVRQAPGKGLVWISRLKSDGSSRSYADFVKGRFTVSRDNVKNTLFLQINSLRAEDTAVYYCARELRGWSIEYWGHGTQVTVSSASTKGGGGSGGGGSGGGGSELVLTQSPATLSLSPGERATLFCRASQDISTSLAWYQQKPGQVPRLLIYGTSYRATGISARFSGRGSGTDFTLTISSLEPEDFALYYCQHRRDWPPGATFGQGTRLEIK

SEQ ID NO: 126 - protein, 220 aa, synthetic construct
DIQLTQSPLSLPVTLGQPASISCRSSQSLVHRNGNTYLHWFQQRPGQSPRLLIYTVSNRFSGVPDRFSGSGSGTDFTLKISRVEAEDVGVYFCSQSSHVPPTFGAGTRLEIKRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 127 - protein, 714 aa, synthetic construct
QVQLQQSGSELKKPGASVKVSCKASGYTFTNYGVNWIKQAPGQGLQWMGWINPNTGEPTFDDDFKGRFAFSLDTSVSTAYLQISSLKADDTAVYFCSRSRGKNEAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKPRGGGGSGGGGSGGGGSQVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKR

SEQ ID NO: 128 - protein, 707 aa, synthetic construct
QVQLQQSGSELKKPGASVKVSCKASGYTFTNYGVNWIKQAPGQGLQWMGWINPNTGEPTFDDDFKGRFAFSLDTSVSTAYLQISSLKADDTAVYFCSRSRGKNEAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKPRGGGGSGGGGSGGGGSEVQLVESGGGLVQPGGSLRLSCAASGFTFSSYGMSWVRQAPGKGLELVASINSNGGSTYYPDSVKGRFTISRDNAKNSLYLQMNSLRAEDTAVYYCASGDYWGQGTTVTVSSGGGGSGGGGSGGGGSDIVMTQSPLSLPVTPGEPASISCRSSQSLVYSNGDTYLHWYLQKPGQSPQLLIYKVSNRFSGVPDRFSGSGSGTDFTLKISRVEAEDVGVYYCSQSTHVPWTFGQGTKVEIKR

SEQ ID NO: 129 - protein, 718 aa, synthetic construct
QVQLQQSGSELKKPGASVKVSCKASGYTFTNYGVNWIKQAPGQGLQWMGWINPNTGEPTFDDDFKGRFAFSLDTSVSTAYLQISSLKADDTAVYFCSRSRGKNEAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKPRGGGGSGGGGSGGGGSEVQLVESGGGLVQPGGSLRLSCSASGFTFSSFGMHWVRQAPGKGLEWVAYISSGSSTIYYGDTVKGRFTISRDNAKNSLFLQMSSLRAEDTAVYYCAREGGYYYGRSYYTMDYWGQGTTVTVSGGGGSGGGGSGGGGSDVVMTQSPLSLPVTPGAPASISCRSSQSIVHSNGNTYLEWYLQKPGQSPKRLLIYKVSNRFSGVPDRFSGSGSGTDFTLRISRVEAEDVGIYYCFQGSHVPPTFGPGTKLEIK

SEQ ID NO: 130 - protein, 716 aa, synthetic construct
QVQLQQSGSELKKPGASVKVSCKASGYTFTNYGVNWIKQAPGQGLQWMGWINPNTGEPTFDDDFKGRFAFSLDTSVSTAYLQISSLKADDTAVYFCSRSRGKNEAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKPRGGGGSGGGGSGGGGSQVQLVESGGGLVQPGGSLRLSCAASGFTFSSYAMSWVRQAPGKGLEWVAINASGTRTYYADSVKGRFTISRDNSKNTLYLQMNLRAEDTAVYYCARGKGNTHKPYGYVRYFDVWGQGTLVTVSSGGGGSGGGGSGGGGSDIVLTQSPATLSLSPGERATLSCRASQSVSSSYLAWYQQKPGQAPRLLIYGASSRATGVPARFSGSGSGTDFTLTISSLEPEDFATYYCLQIYNMPITFGQGTKVEIKRT

SEQ ID NO: 131 - protein, 713 aa, synthetic construct
QVQLQQSGSELKKPGASVKVSCKASGYTFTNYGVNWIKQAPGQGLQWMGWINPNTGEPTFDDDFKGRFAFSLDTSVSTAYLQISSLKADDTAVYFCSRSRGKNEAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKPRGGGGSGGGGSGGGGSEVQLLESGGGLVQPGGSLRLSCTASGFVFSDYWMHWVRQAPGKGLVWISRLKSDGSSRSYADFVKGRFTVSRDNVKNTLFLQINSLRAEDTAVYYCARELRGWSIEYWGHGTQVTVSSASTKGGGGSGGGGSGGGGSELVLTQSPATLSLSPGERATLFCRASQDISTSLAWYQQKPGQVPRLLIYGTSYRATGISARFSGRGSGTDFTLTISSLEPEDFALYYCQHRRDWPPGATFGQGTRLEIK

SEQ ID NO: 132 - protein, 847 aa, synthetic construct
MHLLGPWLLLLVLEYLAFSDSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESKTEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRRVAVGLGSCLAILILAICGLKLQRRWKRTQSQQGLQENSSGQSFFVRNKKVRRAPLSEGPHSLGCYNPMMEDGISYTTLRFPEMNIPRTGDAESSEMQRPPPDCDDTVTYSALHKRQVGDYENVIPDFPEDEGIHYSELIQFGVGERPQAQENVDYVILKH

SEQ ID NO: 133 - protein, 364 aa, synthetic construct
MPLLLLLPLLWAGALAMDPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 134 - protein, 296 aa, synthetic construct
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKVLTKCQEEVSHIPAVHPGSFRPKCDENGNYLPLQCYGSIGYCWCVFPNGTEVPNTRSRGHHNCSESLELEDPSSGLGVTKQDLGPVPM

SEQ ID NO: 135 - protein, 279 aa, synthetic construct
RPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQY

SEQ ID NO: 136 - protein, 112 aa, synthetic construct
DIVLTQTPASLAVSLGQRATISCKASQSVDYDGDSYMNWYQQKPGQPPKLLIYAASNLESGIPARFSGSGSGTDFTLNIHPVEEEDAATYYCQQSNEDPFTFGGGTKLEIKR

SEQ ID NO: 137 - protein, 117 aa, synthetic construct
QVQLQQSGPELVKPGASVKMSCKASGYTFTNYVTHWVKQKPGQGLEWIGYINPYSDGSKYNEKFKGKATLTSDESSSTAYMELSSLTSEDSAVYYCARGKTEWFPYWGQGTTVTVSS

SEQ ID NO: 138 - protein, 776 aa, synthetic construct
MHLLGPWLLLLVLEYLAFSDSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESKTEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRRAEPKSCDKTHTCPPCPGERVQLAHHFSEPEITLIIFGVMAGVIGTILLISYGIRRLIKKSPSDVKPLPSPDTDVPLSSVEIENPETSDQ

SEQ ID NO: 139 - protein, 735 aa, synthetic construct
MHLLGPWLLLLVLEYLAFSDSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESKTEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRRAEPKSCDKTHTCPPCPLTTSGIVTMSHQALGFTLTGLLGTLVTMGLLT

SEQ ID NO: 140 - protein, 13 aa, synthetic construct
CLLNFSCYGYPIQ

SEQ ID NO: 141 - protein, 22 aa, synthetic construct
VFTRSELKFSPQWSHHGKIVTC

SEQ ID NO: 142 - protein, 5 aa, synthetic construct
GGGGS

SEQ ID NO: 143 - protein, 5 aa, synthetic construct
EAAAK

SEQ ID NO: 144 - protein, 5 aa, synthetic construct
PAPAP

SEQ ID NO: 145 - protein, 23 aa, synthetic construct
DIQLTQSPSSLSASVGDRVTISC

SEQ ID NO: 146 - protein, 15 aa, synthetic construct
WYQQKPGKAPKLLIY

SEQ ID NO: 147 - protein, 32 aa, synthetic construct
GVPSRFSGSGSGTEFTLTISSLQPEDFATYFC

SEQ ID NO: 148 - protein, 10 aa, synthetic construct
FGGGTKVEIK

SEQ ID NO: 149 - protein, 30 aa, synthetic construct
EVQLVESGGGLVQPGGSLRLSCAASGFSFS

SEQ ID NO: 150 - protein, 14 aa, synthetic construct
WVRQAPGKGLEWVA

SEQ ID NO: 151 - protein, 32 aa, synthetic construct
RFTISRDNAKNSLYLQMNSLRVEDTALYYCAR

SEQ ID NO: 152 - protein, 11 aa, synthetic construct
WGQGTLVTVSS

SEQ ID NO: 153 - protein, 23 aa, synthetic construct
DIQLTQTTSSLSASLGDRVTISC

SEQ ID NO: 154 - protein, 15 aa, synthetic construct
WYQQKPDGTVKLLIY

SEQ ID NO: 155 - protein, 32 aa, synthetic construct
GVPSRFSGSGSGTDYSLTISNLEQEDFATYFC

SEQ ID NO: 156 - protein, 11 aa, synthetic construct
FGGGTKLEIKR

SEQ ID NO: 157 - protein, 30 aa, synthetic construct
QVQLQESGGGLVKPGGSLKLSCAASGFAFS

SEQ ID NO: 158 - protein, 14 aa, synthetic construct
WVRQTPEKRLEWVA

SEQ ID NO: 159 - protein, 32 aa, synthetic construct
RFTISRDNAKNTLYLQMSSLKSEDTAMYYCAR

SEQ ID NO: 160 - protein, 11 aa, synthetic construct
WGQGTLVTVSS

SEQ ID NO: 161 - protein, 12 aa, synthetic construct
GSAGSAAGSGEF

SEQ ID NO: 162 - protein, 18 aa, synthetic construct
KESGSVSSEQLAQFRSLD

SEQ ID NO: 163 - protein, 13 aa, synthetic construct
EGKSSGSGSESKS

SEQ ID NO: 164 - protein, 27 aa, synthetic construct
SSGNSNANSRGPSFSSGLVPLSLRGSH

SEQ ID NO: 165 - protein, 6 aa, synthetic construct
GGGGGG

SEQ ID NO: 166 - protein, 7 aa, synthetic construct
PAPAPAP

SEQ ID NO: 167 - protein, 9 aa, synthetic construct
PAPAPAPAP

SEQ ID NO: 168 - protein, 11 aa, synthetic construct
PAPAPAPAPAP

(19) United States
(12) Patent Application Publication
(10) Pub. No.: US 2025/0282852 A1
(43) Pub. Date: Sep. 11, 2025

(54) METHODS OF TREATING NEUROLOGICAL DISEASES

(71) Applicant: SINOMAB BIOSCIENCE LIMITED, Pak Shek Kok (NT), Hong Kong (CN)

(72) Inventors: Chi Ho CHONG, Pak Shek Kok (NT), Hong Kong (CN); Shui On LEUNG, Pak Shek Kok (NT), Hong Kong (CN)

(73) Assignee: SINOMAB BIOSCIENCE LIMITED, Pak Shek Kok (NT), Hong Kong (CN)

(21) Appl. No.: 18/858,323

(22) PCT Filed: Apr. 18, 2023
(86) PCT No.: PCT/CN2023/088863

§ 371 Date: Oct. 20, 2024

Related U.S. Application Data
(60) Provisional application No. 63/333,308, filed on Apr. 21, 2022.

(51) Int. Cl. C07K 16/18; A61P 25/28; C07K 16/28

(52) U.S. Cl. CPC C07K 16/18; A61P 25/28; C07K 16/2803; C07K 16/2833; C07K 2317/31; C07K 2317/565; C07K 2317/73; C07K 2317/77

(57) ABSTRACT

Provided herein are methods of promoting removal of beta-amyloid (Aβ) plaque, methods of reducing neuroinflammation, and methods treating a neurological disorder (e.g., Alzheimer's Disease) with certain bispecific antibodies or antigen-binding fragments with one specificity against an internalizing antigen expressed on the surface of neurological cells and the other specificity against a toxic form of Aβ protein. Exemplary antibodies, characteristics thereof, and methods of screening for additional therapeutic bispecific antibodies are also described herein.

### 1. PRIORITY

This application claims priority to U.S. Provisional Application No. 63/333,308 filed Apr. 21, 2022, the entire contents of which are incorporated by reference herein.

### 2. SEQUENCE LISTING

The instant application contains a Sequence Listing compliant with WIPO Standard ST. 26 entitled “SBLO10PCTSL.xml” created Mar. 29, 2023 that is 171,282 bytes in size and hereby incorporated by reference in its entirety.

### 3. FIELD OF THE PRESENT INVENTION

The present invention relates to molecular biology and neurobiology, specifically, to the identification and uses of antibodies in the treatment of various neurological disorders relating to amyloid-β and/or neuroinflammation, such as Alzheimer's disease, amyloidoses and β-amyloid pathology.

### 4. BACKGROUND OF THE PRESENT INVENTION

Alzheimer's disease (AD) is a progressive neurodegenerative disorder characterized by amyloid-β (Aβ)-containing extracellular plaque. The causes leading to the conditions are generally unknown. It has been shown that removal of Aβ plaque and probably other forms, including but not limited to, soluble oligomer, protofibril, fibril, N-terminal truncated form, and post-translational modified form, of Aβ that trigger the formation of plaques, can lead to clinical benefits in AD patients. However, there are only limited therapeutic options available for Aβ plaque removal, which are commonly associated with vascular side effects such as ARIA-E and ARIA-H. Accordingly, there are great needs for novel methods to remove Aβ plaques more efficiently and for therapeutic options for AD with less side effects. Methods provided in the present disclosure meet this need and provide related advantages.

### 5. SUMMARY OF THE PRESENT INVENTION

Provided herein are method of promoting removal of beta-amyloid (Aβ) plaque and probably other forms, including but not limited to soluble oligomer, protofibril, fibril, N-terminal truncated form, and post-translational modified form, of Aβ that trigger the formation of plaques (collectively referred to as Aβ) in a subject in need thereof, comprising administering to the subject a therapeutically effective amount of a bispecific antibody or antigen-binding fragment thereof that specifically binds to surface receptors or antigens expressed on neurons, glia or endothelial cells on one end, and Aβ on the other end wherein the surface receptor/antigen binding portion of the bispecific antibody or antigen-binding fragment (a) induces internalization of the surface receptors/antigens and/or (b) suppresses microglia, astrocyte, oligodendrocyte, pericyte brain endothelial cell or other neuro-immunological cell activation which promote neuro-inflammation and/or (c) reduces pathogenic synaptic pruning by microglia, astrocyte or other neuro-immunological cell and/or (d) modulate antigen presentation by microglia, astrocyte, endothelial cell, or other neuro-immunological cell to infiltrating T cells and/or (c) promote remyelination of damaged myelin sheath by oligodendrocyte or other neuro-immunological cells.

Provided herein are also method of reducing neuroinflammation in a subject in need thereof, comprising administering to the subject a therapeutically effective amount of a bispecific antibody or antigen-binding fragment thereof that specifically binds to surface receptors or antigens expressed on neurological cells on one end and Aβ on the other end wherein the surface receptor/antigen binding portion of the bispecific antibody or antigen-binding fragment (a) induces internalization of the surface receptors/antigens and/or (b) suppresses microglia, astrocyte, oligodendrocyte, pericyte brain endothelial cell or other neuro-immunological cell activation which promote neuro-inflammation and/or (c) reduces pathogenic synaptic pruning by microglia, astrocyte or other neuro-immunological cell and/or (d) modulate antigen presentation by microglia, astrocyte, endothelial cell, or other neuro-immunological cell to infiltrating T cells and/or (c) promote remyelination of damaged myelin sheath by oligodendrocyte or other neuro-immunological cells.

In some embodiments of the methods provided herein, the subject has clinical or pre-clinical Alzheimer's disease, prodromal Alzheimer's disease, Down's syndrome, clinical or pre-clinical amyloid angiopathy (CAA), Parkinson's disease, multi-infarct dementia, cerebral amyloid angiopathy, glaucoma, pre-eclampsia, cognitive impairment, memory loss, or a vascular disorder caused by pathogenic Aβ peptide in blood vessels.

Provided herein are also methods of treating an Aβ-related disease or disorder in a subject in need thereof, comprising administering to the subject a therapeutically effective amount of a bispecific antibody or antigen-binding fragment thereof that specifically binds to surface receptors or proteins expressed on neurological cells on one end and Aβ on the other, wherein the surface receptor/antigen binding portion of the bispecific antibody or antigen-binding fragment targeting neurological cells (a) induces internalization of the surface receptors/antigens and/or (b) suppresses microglia, astrocyte, oligodendrocyte, pericyte brain endothelial cell or other neuro-immunological cell activation which promotes neuro-inflammation and/or (c) reduces pathogenic synaptic pruning by microglia, astrocyte or other neuro-immunological cell and/or (d) modulates antigen presentation by microglia, astrocyte, endothelial cell, or other neuro-immunological cell to infiltrating T cells and/or (c) promotes remyelination of damaged myelin sheath by oligodendrocyte or other neuro-immunological cells.

Provided herein are also methods of treating a disease or disorder associated with neuroinflammation in a subject in need thereof, comprising administering to the subject a therapeutically effective amount of a bispecific antibody or antigen-binding fragment thereof that specifically binds to surface receptors or antigens expressed on neurological cells on one end and Aβon the other, wherein the surface receptor/antigen binding portion of the bispecific antibody or antigen-binding fragment targeting neurological cells (a) induces internalization of the surface receptors/antigens and/or (b) suppresses microglia, astrocyte, oligodendrocyte, pericyte brain endothelial cell or other neuro-immunological cell activation which promote neuro-inflammation and/or (c) reduces pathogenic synaptic pruning by microglia, astrocyte or other neuro-immunological cell and/or (d) modulate antigen presentation by microglia, astrocyte, endothelial cell, or other neuro-immunological cell to infiltrating T cells and/or (c) promote remyelination of damaged myelin sheath by oligodendrocyte or other neuro-immunological cells.

In some embodiments, the Aβ-related or neuroinflammation-related disease or disorder is clinical or pre-clinical Alzheimer's disease, prodromal Alzheimer's disease, Down's syndrome, clinical or pre-clinical amyloid angiopathy (CAA), Parkinson's disease, multi-infarct dementia, cerebral amyloid angiopathy, glaucoma, pre-eclampsia, cognitive impairment, memory loss, or a vascular disorder caused by pathogenic Aβ peptide in blood vessels. In some embodiments, the Aβ-related disease or disorder is Alzheimer's disease.

In some embodiments, the antibody or antigen-binding fragment used in the methods described herein is a bispecific antibody or antigen-binding fragment.

In some embodiments, the bispecific antibody or antigen-binding fragment used in the methods disclosed herein is a bispecific antibody in a format selected from the group consisting of an IgG1 antibody, an IgG2 antibody, an IgG3 antibody, and an IgG4 antibody. In some embodiments, the bispecific antibody is in the format of an IgG1 antibody.

In some embodiments, the bispecific antibody or antigen-binding fragment used in the methods disclosed herein comprises the surface receptor/antigen binding portion in an antibody format selected from the group consisting of an IgG1 antibody, an IgG2 antibody, an IgG3 antibody, and an IgG4 antibody, whereas the Aβ-binding antibody is in the form of a single chain Fv (scFv). In some embodiments, the bispecific antibody is in the form of an IgG1 antibody.

In some embodiments, the bispecific antibody or antigen-binding fragment used in the methods disclosed herein comprises the Aβ-binding portion in an antibody format selected from the group consisting of an IgG1 antibody, an IgG2 antibody, an IgG3 antibody, and an IgG4 antibody, whereas the surface receptor/antigen-bind ing antibody is in the form of a single chain Fv (scFv). In some embodiments, the bispecific antibody is in the form of an IgG1 antibody.

In some embodiments, the bispecific antibody or antigen-binding fragment used in the methods disclosed herein is selected from the group consisting of a Fab, a Fab′, a F (ab′) 2, a Fv, a scFv, a (scFv) 2, a single domain antibody (sdAb), and a heavy chain antibody (HCAb).

In some embodiments, the bispecific antibody or antigen-binding used in the methods disclosed herein is a chimeric antibody or antigen-binding, a humanized antibody or antigen-binding, or a human antibody or antigen-binding.

In some embodiments, the bispecific antibody or antigen-binding fragment used in methods disclosed herein specifically binds to surface receptors or antigens expressed on neurological cells on one end and Aβ protein on the other.

In some embodiments, the bispecific antibody or antigen-binding fragment used in methods disclosed herein specifically binds to surface receptors or antigens expressed on microglia cells on one end and Aβ protein on the other.

In some embodiments, the bispecific antibody or antigen-binding fragment used in methods disclosed herein specifically binds to human CD22 expressed on neurological cells on one end and Aβprotein on the other.

In some embodiments, the bispecific antibody or antigen-binding fragment used in methods disclosed herein specifically binds to CLLNFSCYGYPIQ (SEQ ID NO: 122) and VFTRSELKFSPQWSHHGKIVTC (SEQ ID NO: 123) of human CD22 expressed on neurological cells on one end and Aβ protein on the other.

In some embodiments, the bispecific antibody or antigen-binding fragment used in methods disclosed herein specifically binds to human CD22 expressed on microglia cells on one end and Aβprotein on the other.

In some embodiments, the bispecific antibody or antigen-binding fragment used in methods disclosed herein specifically binds to CLLNFSCYGYPIQ (SEQ ID NO: 122) and VFTRSELKFSPQWSHHGKIVTC (SEQ ID NO: 123) of human CD22 expressed on microglia cells on one end and Aβ protein on the other.

In some embodiments, the bispecific antibody or antigen-binding fragment used in methods disclosed herein specifically binds to human CD33 in neurological cells on one end and Aβ protein on the other.

In some embodiments, the bispecific antibody or antigen-binding fragment used in methods disclosed herein specifically binds to human CD33 in microglia cells on one end and Aβ protein on the other.

In some embodiments, the bispecific antibody or antigen-binding fragment used in methods disclosed herein specifically binds to human CD74 in neurological cells on one end and Aβ protein on the other.

In some embodiments, the bispecific antibody or antigen-binding fragment used in methods disclosed herein specifically binds to human CD74 in microglia cells on one end and Aβ protein on the other.

In some embodiments, the surface receptor/antigen binding portion of the bispecific antibody or antigen-binding fragment used in methods disclosed herein comprises a light chain variable region (VL) comprising a VL CDR1, VL CDR2, and VL CDR3 that have the amino acid sequences of SEQ ID NO: 1, SEQ ID NO: 2, and SEQ ID NO: 3, respectively; and a heavy chain variable region (VH) comprising a VH CDR1, VH CDR2, and VH CDR3 that have the amino acid sequence of SEQ ID NO: 4, SEQ ID NO: 5, and SEQ ID NO: 6, respectively.

In some embodiments, the surface receptor/antigen binding portion of the bispecific antibody or antigen-binding fragment used in methods disclosed herein is a chimeric antibody or antigen-binding fragment. In some embodiments, the VL and VH of the chimeric antibody or antigen-binding fragment have the amino acid sequences of SEQ ID NO: 7 and SEQ ID NO: 8, respectively. In some embodiments, the chimeric antibody or antigen-binding fragment is SM03.

In some embodiments, the surface receptor/antigen binding portion of the bispecific antibody or antigen-binding fragment used in methods disclosed herein is a humanized antibody or antigen-binding fragment. In some embodiments, the VL and VH of the humanized antibody or antigen-binding fragment have the amino acid sequences of SEQ ID NO: 9 and SEQ ID NO: 10, respectively. In some embodiments, the humanized antibody or antigen-binding fragment is SM06.

In some embodiments, the surface receptor/antigen binding portion of the bispecific antibody or antigen-binding fragment used in methods disclosed herein comprises a light chain variable region (VL) comprising a VL CDR1, VL CDR2, and VL CDR3 that have the amino acid sequences of SEQ ID NO: 11, SEQ ID NO: 12, and SEQ ID NO: 13, respectively; and a heavy chain variable region (VH) comprising a VH CDR1, VH CDR2, and VH CDR3 that have the amino acid sequence of SEQ ID NO: 14, SEQ ID NO: 15, and SEQ ID NO: 16, respectively.

In some embodiments, the surface receptor/antigen binding portion of the bispecific antibody or antigen-binding fragment used in methods disclosed herein is a chimeric antibody or antigen-binding fragment. In some embodiments, the VL and VH of the chimeric antibody or antigen-binding fragment have the amino acid sequences of SEQ ID NO: 17 and SEQ ID NO: 18, respectively. In some embodiments, the chimeric antibody or antigen-binding fragment is LL2.

In some embodiments, the surface receptor/antigen binding portion of the bispecific antibody or antigen-binding fragment used in methods disclosed herein is a humanized antibody or antigen-binding fragment specific for human CD22. In some embodiments, the VL and VH of the humanized antibody or antigen-binding fragment have the amino acid sequences of SEQ ID NO: 19 and SEQ ID NO: 20, respectively. In some embodiments, the humanized antibody or antigen-binding fragment is hLL2.

In some embodiments, the receptor binding portion of the bispecific antibody or antigen-binding fragment used in methods disclosed herein comprises a light chain variable region (VL) comprising a VL CDR1, VL CDR2, and VL CDR3 that have the amino acid sequences of SEQ ID NO: 21, SEQ ID NO: 22, and SEQ ID NO: 23, respectively; and a heavy chain variable region (VH) comprising a VH CDR1, VH CDR2, and VH CDR3 that have the amino acid sequence of SEQ ID NO: 24, SEQ ID NO: 25, and SEQ ID NO: 26, respectively.

In some embodiments, the surface receptor or antigen binding portion of the bispecific antibody or antigen-binding fragment used in methods disclosed herein is a humanized antibody or antigen-binding fragment. In some embodiments, the VL and VH of the humanized antibody or antigen-binding fragment have the amino acid sequences of SEQ ID NO: 27 and SEQ ID NO: 28, respectively. In some embodiments, the humanized antibody or antigen-binding fragment is Gemtuzumab.

In some embodiments, the receptor binding portion of the bispecific antibody or antigen-binding fragment used in methods disclosed herein comprises a light chain variable region (VL) comprising a VL CDR1, VL CDR2, and VL CDR3 that have the amino acid sequences of SEQ ID NO: 29, SEQ ID NO: 30, and SEQ ID NO: 31, respectively; and a heavy chain variable region (VH) comprising a VH CDR1, VH CDR2, and VH CDR3 that have the amino acid sequence of SEQ ID NO: 32, SEQ ID NO: 33, and SEQ ID NO: 34 respectively.

In some embodiments, the surface receptor or antigen binding portion of the bispecific antibody or antigen-binding fragment used in methods disclosed herein is a humanized antibody or antigen-binding fragment. In some embodiments, the VL and VH of the humanized antibody or antigen-binding fragment have the amino acid sequences of SEQ ID NO: 35 and SEQ ID NO: 36, respectively. In some embodiments, the humanized antibody or antigen-binding fragment is HuMy9-6.

In some embodiments, the receptor binding portion of the bispecific antibody or antigen-binding fragment used in methods disclosed herein comprises a light chain variable region (VL) comprising a VL CDR1, VL CDR2, and VL CDR3 that have the amino acid sequences of SEQ ID NO: 37, SEQ ID NO: 38, and SEQ ID NO: 39, respectively; and a heavy chain variable region (VH) comprising a VH CDR1, VH CDR2, and VH CDR3 that have the amino acid sequence of SEQ ID NO: 40, SEQ ID NO: 41, and SEQ ID NO: 42, respectively.

In some embodiments, the surface receptor or antigen binding portion of the bispecific antibody or antigen-binding fragment used in methods disclosed herein is a humanized antibody or antigen-binding fragment. In some embodiments, the VL and VH of the humanized antibody or antigen-binding fragment have the amino acid sequences of SEQ ID NO: 43 and SEQ ID NO: 44, respectively. In some embodiments, the humanized antibody or antigen-binding fragment is Lintuzumab.

In some embodiments, the receptor binding portion of the bispecific antibody or antigen-binding fragment used in methods disclosed herein comprises a light chain variable region (VL) comprising a VL CDR1, VL CDR2, and VL CDR3 that have the amino acid sequences of SEQ ID NO: 45, SEQ ID NO: 46, and SEQ ID NO: 47, respectively; and a heavy chain variable region (VH) comprising a VH CDR1, VH CDR2, and VH CDR3 that have the amino acid sequence of SEQ ID NO: 48, SEQ ID NO: 49, and SEQ ID NO: 50 respectively.

In some embodiments, the surface receptor or antigen binding portion of the bispecific antibody or antigen-binding fragment used in methods disclosed herein is a chimeric antibody or antigen-binding fragment. In some embodiments, the VL and VH of the chimeric antibody or antigen-binding fragment have the amino acid sequences of SEQ ID NO: 51 and SEQ ID NO: 52, respectively. In some embodiments, the chimeric antibody or antigen-binding fragment is LL1.

In some embodiments, the surface receptor or antigen binding portion of the bispecific antibody or antigen-binding fragment used in methods disclosed herein is a humanized antibody or antigen-binding fragment. In some embodiments, the VL and VH of the humanized antibody or antigen-binding fragment have the amino acid sequences of SEQ ID NO: 53 and SEQ ID NO: 54, respectively. In some embodiments, the humanized antibody or antigen-binding fragment is hLL1.

In some embodiments, the Amyloid β binding portion of the bispecific antibody or antigen-binding fragment used in methods disclosed herein comprises a light chain variable region (VL) comprising a VL CDR1, VL CDR2, and VL CDR3 that have the amino acid sequences of SEQ ID NO: 55, SEQ ID NO: 56, and SEQ ID NO: 57, respectively; and a heavy chain variable region (VH) comprising a VH CDR1, VH CDR2, and VH CDR3 that have the amino acid sequence of SEQ ID NO: 58, SEQ ID NO: 59, and SEQ ID NO: 60 respectively.

In some embodiments, the Amyloid β or antigen binding portion of the bispecific antibody or antigen-binding fragment used in methods disclosed herein is a humanized antibody or antigen-binding fragment. In some embodiments, the VL and VH of the humanized antibody or antigen-binding fragment have the amino acid sequences of SEQ ID NO: 61 and SEQ ID NO: 62 or SEQ ID NO: 63, respectively. In some embodiments, the humanized antibody or antigen-binding fragment is Aducanumab.

In some embodiments, the Amyloid β binding portion of the bispecific antibody or antigen-binding fragment used in methods disclosed herein comprises a light chain variable region (VL) comprising a VL CDR1, VL CDR2, and VL CDR3 that have the amino acid sequences of SEQ ID NO: 64, SEQ ID NO: 65, and SEQ ID NO: 66, respectively; and a heavy chain variable region (VH) comprising a VH CDR1, VH CDR2, and VH CDR3 that have the amino acid sequence of SEQ ID NO: 67, SEQ ID NO: 68, and SEQ ID NO: 69 respectively.

In some embodiments, the Amyloid β or antigen binding portion of the bispecific antibody or antigen-binding fragment used in methods disclosed herein is a humanized antibody or antigen-binding fragment. In some embodiments, the VL and VH of the humanized antibody or antigen-binding fragment have the amino acid sequences of SEQ ID NO: 70 and SEQ ID NO: 71, respectively. In some embodiments, the humanized antibody or antigen-binding fragment is BAN2401.

In some embodiments, the Amyloid β portion of the bispecific antibody or antigen-binding fragment used in methods disclosed herein comprises a light chain variable region (VL) comprising a VL CDR1, VL CDR2, and VL CDR3 that have the amino acid sequences of SEQ ID NO: 72, SEQ ID NO: 73, and SEQ ID NO: 74, respectively; and a heavy chain variable region (VH) comprising a VH CDR1, VH CDR2, and VH CDR3 that have the amino acid sequence of SEQ ID NO: 75, SEQ ID NO: 76, and SEQ ID NO: 77 respectively.

In some embodiments, the Amyloid β or antigen binding portion of the bispecific antibody or antigen-binding fragment used in methods disclosed herein is a humanized antibody or antigen-binding fragment. In some embodiments, the VL and VH of the humanized antibody or antigen-binding fragment have the amino acid sequences of SEQ ID NO: 78 and SEQ ID NO: 79, respectively. In some embodiments, the humanized antibody or antigen-binding fragment is Gantenerumab.

In some embodiments, the Amyloid β binding portion of the bispecific antibody or antigen-binding fragment used in methods disclosed herein comprises a light chain variable region (VL) comprising a VL CDR1, VL CDR2, and VL CDR3 that have the amino acid sequences of SEQ ID NO: 80, SEQ ID NO: 81, and SEQ ID NO: 82, respectively; and a heavy chain variable region (VH) comprising a VH CDR1, VH CDR2, and VH CDR3 that have the amino acid sequence of SEQ ID NO: 83, SEQ ID NO: 84, and SEQ ID NO: 85 respectively.

In some embodiments, the Amyloid β or antigen binding portion of the bispecific antibody or antigen-binding fragment used in methods disclosed herein is a humanized antibody or antigen-binding fragment. In some embodiments, the VL and VH of the humanized antibody or antigen-binding fragment have the amino acid sequences of SEQ ID NO: 86 and SEQ ID NO: 87, respectively. In some embodiments, the humanized antibody or antigen-binding fragment is Crenczumab.

In some embodiments, the Amyloid β binding portion of the bispecific antibody or antigen-binding fragment used in methods disclosed herein comprises a light chain variable region (VL) comprising a VL CDR1, VL CDR2, and VL CDR3 that have the amino acid sequences of SEQ ID NO: 88, SEQ ID NO: 89, and SEQ ID NO: 90, respectively; and a heavy chain variable region (VH) comprising a VH CDR1, VH CDR2, and VH CDR3 that have the amino acid sequence of SEQ ID NO: 91, SEQ ID NO: 92, and SEQ ID NO: 93 respectively.

In some embodiments, the Amyloid β or antigen binding portion of the bispecific antibody or antigen-binding fragment used in methods disclosed herein is a humanized antibody or antigen-binding fragment. In some embodiments, the VL and VH of the humanized antibody or antigen-binding fragment have the amino acid sequences of SEQ ID NO: 94 and SEQ ID NO: 95, respectively. In some embodiments, the humanized antibody or antigen-binding fragment is AD38.

In some embodiments, the antigen binding portion of the bispecific antibody or antigen-binding fragment used in methods disclosed herein is a humanized antibody or antigen-binding fragment. In some embodiments, the light chain and heavy chain of the humanized antibody or antigen-binding fragment have the amino acid sequences of SEQ ID NO: 96 and SEQ ID NO: 97, respectively. In some embodiments, the humanized antibody or antigen-binding fragment is SM03-crc.

In some embodiments, the antigen binding portion of the bispecific antibody or antigen-binding fragment used in methods disclosed herein is a humanized antibody or antigen-binding fragment. In some embodiments, the light chain and heavy chain of the humanized antibody or antigen-binding fragment have the amino acid sequences of SEQ ID NO: 96 and SEQ ID NO: 98, respectively. In some embodiments, the humanized antibody or antigen-binding fragment is SM03-ban.

In some embodiments, the antigen binding portion of the bispecific antibody or antigen-binding fragment used in methods disclosed herein is a humanized antibody or antigen-binding fragment. In some embodiments, the light chain and heavy chain of the humanized antibody or antigen-binding fragment have the amino acid sequences of SEQ ID NO: 96 and SEQ ID NO: 99, respectively. In some embodiments, the humanized antibody or antigen-binding fragment is SM03-gan.

In some embodiments, the antigen binding portion of the bispecific antibody or antigen-binding fragment used in methods disclosed herein is a humanized antibody or antigen-binding fragment. In some embodiments, the light chain and heavy chain of the humanized antibody or antigen-binding fragment have the amino acid sequences of SEQ ID NO: 96 and SEQ ID NO: 100, respectively. In some embodiments, the humanized antibody or antigen-binding fragment is SM03-adu.

In some embodiments, the antigen binding portion of the bispecific antibody or antigen-binding fragment used in methods disclosed herein is a humanized antibody or antigen-binding fragment. In some embodiments, the light chain and heavy chain of the humanized antibody or antigen-binding fragment have the amino acid sequences of SEQ ID NO: 101 and SEQ ID NO: 102, respectively. In some embodiments, the humanized antibody or antigen-binding fragment is SM06-ban.

In some embodiments, the antigen binding portion of the bispecific antibody or antigen-binding fragment used in methods disclosed herein is a humanized antibody or antigen-binding fragment. In some embodiments, the light chain and heavy chain of the humanized antibody or antigen-binding fragment have the amino acid sequences of SEQ ID NO: 101 and SEQ ID NO: 103, respectively. In some embodiments, the humanized antibody or antigen-binding fragment is SM06-gan.

In some embodiments, the antigen binding portion of the bispecific antibody or antigen-binding fragment used in methods disclosed herein is a humanized antibody or antigen-binding fragment. In some embodiments, the light chain and heavy chain of the humanized antibody or antigen-binding fragment have the amino acid sequences of SEQ ID NO: 101 and SEQ ID NO: 104, respectively. In some embodiments, the humanized antibody or antigen-binding fragment is SM06-adu.

In some embodiments, the antigen binding portion of the bispecific antibody or antigen-binding fragment used in methods disclosed herein is a humanized antibody or antigen-binding fragment. In some embodiments, the light chain and heavy chain of the humanized antibody or antigen-binding fragment have the amino acid sequences of SEQ ID NO: 101 and SEQ ID NO: 105, respectively. In some embodiments, the humanized antibody or antigen-binding fragment is SM06-ad38.

In some embodiments, the antigen binding portion of the bispecific antibody or antigen-binding fragment used in methods disclosed herein is a humanized antibody or antigen-binding fragment. In some embodiments, the light chain and heavy chain of the humanized antibody or antigen-binding fragment have the amino acid sequences of SEQ ID NO: 106 and SEQ ID NO: 107, respectively. In some embodiments, the humanized antibody or antigen-binding fragment is Gem-adu.

In some embodiments, the antigen binding portion of the bispecific antibody or antigen-binding fragment used in methods disclosed herein is a humanized antibody or antigen-binding fragment. In some embodiments, the light chain and heavy chain of the humanized antibody or antigen-binding fragment have the amino acid sequences of SEQ ID NO: 108 and SEQ ID NO: 109, respectively. In some embodiments, the humanized antibody or antigen-binding fragment is 96-adu.

In some embodiments, the antigen binding portion of the bispecific antibody or antigen-binding fragment used in methods disclosed herein is a humanized antibody or antigen-binding fragment. In some embodiments, the light chain and heavy chain of the humanized antibody or antigen-binding fragment have the amino acid sequences of SEQ ID NO: 110 and SEQ ID NO: 111, respectively. In some embodiments, the humanized antibody or antigen-binding fragment is Lin-adu.

In some embodiments, the antigen binding portion of the bispecific antibody or antigen-binding fragment used in methods disclosed herein is a humanized antibody or antigen-binding fragment. In some embodiments, the light chain and heavy chain of the humanized antibody or antigen-binding fragment have the amino acid sequences of SEQ ID NO: 112 and SEQ ID NO: 113, respectively. In some embodiments, the humanized antibody or antigen-binding fragment is hLL1-adu.

In some embodiments of the methods disclosed herein, the bispecific antibody or antigen-binding fragment is administered intravenously, intramuscularly, subcutaneously, intracranially, intrathecally, intraventricularly, intraperitoneally, intranasally, parenterally, topically, or intradermally. In some embodiments, the bispecific antibody or antigen-binding fragment is administered intravenously. In some embodiments, the bispecific antibody or antigen-binding fragment is administered subcutaneously.

In some embodiments of the methods disclosed herein, the bispecific antibody or antigen-binding fragment is administered in a therapeutically effective amount within the range of 1-50 mg/kg of body weight of the subject. In some embodiments, therapeutically effective amount is about 1, about 2, about 3, about 5, about 10, about 15, or about 30 mg/kg of body weight of the subject. In some embodiments, the bispecific antibody or antigen-binding fragment is administered in a therapeutically effective amount at 300-1,200 mg per dose. In some embodiments, the bispecific antibody or antigen-binding fragment is administered biweekly or monthly. In some embodiments, the bispecific antibody or antigen-binding fragment is administered in multiple doses. In some embodiments, the bispecific antibody or antigen-binding fragment is administered in multiple doses over a period of at least three months, at least six months, or at least one year.

In some embodiments of the methods disclosed herein, the bispecific antibody or antigen-binding fragment is administered in combination with a second therapeutic agent. In some embodiments, the second therapeutic agent is an anti-beta-amyloid antibody, an anti-CD22 antibody, an anti-CD33 antibody, an anti-CD74 antibody, a Tau aggregation inhibitor, a Tau protein modulator, a cholinesterase inhibitor, an acetylcholinesterase inhibitor, an N-methyl D-aspartate (NMDA) antagonist, a β-secretase inhibitor, or an insulin sensitizer.

In some embodiments, the second therapeutic agent is an anti-beta-amyloid antibody selected from the group consisting of aducanumab, donanemab, gantenerumab, lecanemab, crenezumab, bapincuzumab, solanezumab and AD-38.

In some embodiments, the second therapeutic agent is an anti-CD22 antibody selected from the group consisting of SM03, SM06, Epratuzumab, Moxctumomab pasudotox, inotuzumab ozogamicin, OXS-1550, SCRI-CAR22v2, MB-CAR-T19-22, CD22-CART, AUTO-3, UCART-22, TRPH-222, JCAR-018, ThisCART22, JNJ-75348780, YT-19/22, AUTO-1-NG, CTA-101, GC-022, LB-1909, MendCART, JJO-686, SCRI-CAR19X22v1, Hb22.7 and anti-CD22-NMS-249.

In some embodiments, the second therapeutic agent is an anti-CD33 antibody selected from the group consisting of AL003, gemtuzumab ozogamicin, lintuzumab, vadastuximab talirine, BI836858, VCAR-33, VOR-33, OXS-3550, AMV-564, cluvixtamab, LB-1910, JNJ-67571244, PRGN-3006, ICG-136, GEM-333,.

In some embodiments, the second therapeutic agent is an anti-CD74 antibody selected from the group consisting of milatuzumab, STRO-001, hLL1-CL2A-SN-38, milatuzumab-Fab-veltuzumab, 74-(20)-(20), 2L-rpRNAse-milatuzumab-g4P, milatuzumab-doxorubicin conjugate, and HuMax-CD74-ADC.

In some embodiments of the methods disclosed herein, the subject is a human subject.

### 6. BRIEF DESCRIPTION OF DRAWINGS

FIGS. 1A and 1B provide flow cytometry binding data showing the expression of CD22 (FIG. 1A) and CD33 and CD74 (FIG. 1B) in human microglia cell line, HMC-3.

FIG. 2 provides the Octet in vitro binding data showing the binding of Aβ1-42 to recombinant CD22 protein. As shown, CD22 binds to oligomeric Aβ1-42 with a K_{D} of 2.79 nM.

FIG. 3 provides SDS-PAGE data on the size of the CD22-Aβ bispecific antibodies SM03-Adu and SM06-Adu.

FIG. 4 provides ELISA data showing the binding of the bispecific antibodies SM03-Adu, SM06-Adu, SM03-BAN, SM06-BAN to both CD22 and Aβ.

FIG. 5 provides flow cytometry data showing the binding of the CD22-AB bispecific antibody SM03-Adu to HMC-3 cells.

FIG. 6 depicts the enhanced inhibition of TNF-α expression by SM03-BAN as compared to either the anti-Aβ antibody (BAN 2401) or the anti-CD22 antibody (SM03) alone.

FIG. 7 illustrates the enhanced suppressive effect of SM03-Adu on IL-6 release as compared to the anti Aβ antibody (Aducanumab) and the anti-CD22 antibody (SM03)

FIG. 8 illustrates the suppression of Aβ-induced IL-1β release in human PBMC by the bispecific antibodies SM03-Adu and SM06-Adu.

FIG. 9 illustrates the Aβ-induced apoptotic cell death of oligodendrocytes by the bispecific antibodies SM03-Adu and SM06-Adu.

FIG. 10 illustrates the rates of internalization of the bispecific antibodies SM03-Adu, SM06-Adu, hLL1-Adu, and Gem-Adu.

FIG. 11 illustrates the Aβ clearance of 2 different version of SM03-Adu as compared to either the anti-Aβ monoclonal antibody (Aducanumab) or SM03 alone.

### 7. DETAILED DESCRIPTION OF THE PREFERRED EMBODIMENTS

Alzheimer's disease (AD) is a progressive neurodegenerative disorder characterized by amyloid-β (Aβ) containing extracellular plaque. Effective therapeutic options are still in great need. Disclosed herein are inventors' surprising discovery that bispecific antibody targeting surface receptors, namely CD22, CD33 or CD74, and oligomeric or fibrillar Aβ1-42 promote the removal of Aβ plaque by inducing internalization of surface receptors. Additionally, it was found that the bispecific antibodies disclosed herein can also suppress autoimmunity in the central nervous system (CNS), providing further therapeutic benefits. Accordingly, in some embodiments, provided herein are methods of reducing Aβ accumulation and neuroinflammation, and treating related disease or disorder (e.g., AD) via a CD22/CD33/CD74-mediated mechanism.

Dementia is estimated to increase from 50 million in 2010 to 113 million by 2050 worldwide. AD is the major cause of dementia and is often complicated with other neurodegenerative and cerebrospinal pathology. The neuropathologie features begin 15 to 20 years before obvious cognitive symptoms. Individuals with AD will progress from their normal baseline cognitive abilities through subtle changes of the preclinical stages, to obvious symptoms of brain dysfunction, termed prodromal AD, and finally, to AD dementia, which eventually impairs ability to perform activities of daily living (ADL) that they were previously independently performing, owing to cognitive deficits. The preclinical stage of AD is often considered the stage occurring before a clinical diagnosis of a cognitive disorder is considered. AD dementia is a synaptic dysfunction disease encompassing changes in molecular, cellular and connectome level. Clinical manifestation of AD patients includes dementia with amnestic or non-amnestic symptoms. This is often accompanied with verbal, visuospatial processing, and executive dysfunction. (Scharre, Practical Neurology (2019); Dubois et al., Alzheimers Dement (2016), 12 (3): 292-323.) Neuropathological hallmark of AD is characterized by excessive neuroinflammation, accumulation of extracellular Aβ containing plaque and hyper-phosphorylated tau protein. Aβ plaque is widespread in cortical and hippocampal region and causes disrupted neuronal network via triggering neuronal loss and promotion of neuroinflammation via microglia.

Aβ is derived by amyloid precursor protein, which is a transmembrane protein enriched in neuronal surface membrane. Physiologically it is cleaved by a-secretase and later γ-secretase to form APPsα. It functions in regulation of synaptic strength via modulating calcium flux and potassium channel. In amyloidogenic pathway, Aβ is cleaved by β-secretase and subsequently γ-secretase to form the Aβ1-42. Aβ1-42 misfolds in a β-sheet conformation and aggregates into toxic oligomeric form. In accordance with the amyloid cascade hypothesis, toxic oligomeric Aβ1-42 aggregate into plaques which lead to neurotoxicity and dementia. Oligomeric Aβ1-42 directly binds to metabotropic glutamate receptor 5 (mGluR5), N-Methyl-D-aspartic acid (NMDA) receptor, and other neuron receptors such as α-7 nicotinic acetylcholine receptor and insulin receptors to induce pathological changes in synaptic strength and morphology of dendritic spines. Oligomeric Aβ1-42 could also stimulate microglia cell via pattern recognition receptors, namely Toll-like receptors (TLRs), Nod-like receptors (NLRs), RIG-like receptors (RLRs), AIM2-like receptors (ALRs), to trigger neuroinflammation. Aβ1-42 induces pro-inflammatory cytokines, namely IL-1β, IL-8 and TNFα, released by microglia. Also, it activates NLRP3 inflammasome cascade to secrete ASC specks protein (apoptosis-associated speck-like protein containing a caspase-1 recruitment domain) for the nucleation of further aggregation of Aβ1-42.

Clearance of toxic oligomeric Aβ has been proposed to render clinical benefits in AD patients. As in 2021, of the 17 disease-modifying therapeutic in Phase III, 29% of them target Aβ as their mechanism of action (MOA). Anti-Aβ antibody, including aducanumab, donanemab, gantenerumab, lecanemab, crenezumab, bapincuzumab and solanezumab target various toxic form of Aβ via Fc receptor-mediated phagocytosis and non-Fc receptor-mediated clearance of Aβ.

Aducanumab is an IgG1 mAb selectively targeting soluble oligomer and insoluble fibrils of Aβ.Aducanumab binds amino acids 3-6 and recognizes conformational epitope on aggregated Aβ but not monomeric form. The binding of the antibody to Aβaggregate triggers clearance by Fc gamma receptor-mediated phagocytosis, restoring calcium homeostasis of neuronal network in AD patients. Aducanumab was approved by FDA in June 2021 for the treatment of AD as the clinical studies consistently displayed reduction of amyloid plaques.

In addition to AD, other disease or disorders associated with abnormal accumulation of Aβ include, for example, Down's syndrome, clinical or pre-clinical amyloid angiopathy (CAA), Parkinson's disease, multi-infarct dementia, cerebral amyloid angiopathy, glaucoma, pre-cclampsia, cognitive impairment, memory loss, or a vascular disorder caused by pathogenic Aβ peptide in blood vessels.

Before the present disclosure is further described, it is to be understood that the disclosure is not limited to the particular embodiments set forth herein, and it is also to be understood that the terminology used herein is for the purpose of describing particular embodiments, and is not intended to be limiting.

Unless otherwise defined herein, scientific and technical terms used in the present disclosures shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, genetics and protein and nucleic acid chemistry and hybridization described herein are those well-known and commonly used in the art.

The term “a” or “an” entity refers to one or more of that entity; for example, “a vector,” is understood to represent one or more vectors.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and B″ (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Exemplary genes and polypeptides are described herein with reference to GenBank numbers, GI numbers and/or SEQ ID NOS. It is understood that one skilled in the art can readily identify homologous sequences by reference to sequence sources, including but not limited to GenBank (ncbi. nlm. nih. gov/genbank/) and EMBL (embl. org/).

### 7.1 Aβ Clearance and Neuroinflammation Reduction

There are several receptor proteins that are found on the surface of neurological cells such as microglia, astrocyte, oligodendrocyte that play a role in regulating the immune system in the CNS. Of particular interest is that some of which, such as human CD22, CD33, and CD74 can also induce receptor internalization. Bispecific antibodies that target these receptors on one end and Aβ protein on the other end will have the dual function of modulating pro-inflammatory activities and inducing Aβ internalization and clearance, thereby achieving enhanced therapeutic effects for the treatment of neurological disorders.

CD22 is a B cell restricted antigen that belongs to the immunoglobulin (Ig) superfamily. CD22 is a type I transmembrane sialoglycoprotein, also known as sialic acid binding immunoglobulin-type lectins (Siglecs). Human CD22 specifically binds to the structure: N-acetylneuraminic acid α (2-6) galactose (NeuAc-α (2-6) Gal), which is known to be expressed on hematopoietic cells, liver cells, lung epithelial cells, splenic cords and follicles, ileum stoma, heart stroma, blood vessels, skin epithelial cell secretions in eccrine sweat glands, colon stromal cells, liver sinusoids and stromal cells, and cells in central nervous system (CNS) such as the microglia cells in brain white matter (Gagneux et al., Journal of Biological Chemistry (2003), 278 (48): 48245-50; Safaiyan et al., Neuron (2021), 109 (7): 1100-17. e10). CD22 binds to its specific ligand distributed either on the same cell (cis-binding), or on a different cell (trans-binding). Cis-binding of CD22 is commonly found between neighboring molecules where the glycan binding site of a CD22 molecule is ligated to the glycan molecule of another CD22 or glycoprotein on the same cell, forming homo-oligomers or homo-multimers. Cis-binding of CD22 is demonstrated to exert a masking effect on the molecule, preventing the Siglec from forming cell to cell ligation (trans-binding). In resting B cells, CD22 is a prominent cis-ligand for itself, forming CD22 homo-oligomers. Trans-binding of CD22 results in the physical association of the Siglec with B-cell receptor (BCR) to exert a maximal inhibitory response, which is required for immune tolerance via eliciting inhibitory immunological signals (Pessutto et al., 1987. J. Immunol. 138:98-103; Doody et al. 1995. Science 269:242-344; Lanoue et al. 2002. Eur. J. Immunol. 32:348-355; Courtney et al. 2009. PNAS 106:2500-2505). Additional information about human CD22, including its exemplary amino acid sequences can be found in public database such as UniProt (UniProt KB/Swiss-Prot ID: P20273) and GENBANK (NCBI Ref. NP_001172028.1, NP_001172029.1, NP_001172030.1, NP_001265346.1, NP_001762.2). An exemplary sequence is also provided below.

| (SEQ ID NO: 114) | |
|---|---|
| 1 | MHLLGPWLLL LVLEYLAFSD SSKWVFEHPE TLYAWEGACV WIPCTYRALD GDLESFILFH |
| 61 | NPEYNKNTSK FDGTRLYEST KDGKVPSEQK RVQFLGDKNK NCTLSIHPVH LNDSGQLGLR |
| 121 | MESKTEKWME RIHLNVSERP FPPHIQLPPE IQESQEVTLT CLLNFSCYGY PIQLQWLLEG |
| 181 | VPMRQAAVTS TSLTIKSVFT RSELKFSPQW SHHGKIVTCQ LQDADGKFLS NDTVQLNVKH |
| 241 | TPKLEIKVTP SDAIVREGDS VTMTCEVSSS NPEYTTVSWL KDGTSLKKQN TFTLNLREVT |
| 301 | KDQSGKYCCQ VSNDVGPGRS EEVFLQVQYA PEPSTVQILH SPAVEGSQVE FLCMSLANPL |
| 361 | PTNYTWYHNG KEMQGRTEEK VHIPKILPWH AGTYSCVAEN ILGTGQRGPG AELDVQYPPK |
| 421 | KVTTVIQNPM PIREGDTVTL SCNYNSSNPS VTRYEWKPHG AWEEPSLGVL KIQNVGWDNT |
| 481 | TIACAACNSW CSWASPVALN VQYAPRDVRV RKIKPLSEIH SGNSVSLQCD FSSSHPKEVQ |
| 541 | FFWEKNGRLL GKESQLNFDS ISPEDAGSYS CWVNNSIGQT ASKAWTLEVL YAPRRLRVSM |
| 601 | SPGDQVMEGK SATLTCESDA NPPVSHYTWF DWNNQSLPYH SQKLRLEPVK VQHSGAYWCQ |
| 661 | GTNSVGKGRS PLSTLTVYYS PETIGRRVAV GLGSCLAILI LAICGLKLQR RWKRTQSQQG |
| 721 | LQENSSGQSF FVRNKKVRRA PLSEGPHSLG CYNPMMEDGI SYTTLRFPEM NIPRTGDAES |
| 781 | SEMQRPPPDC DDTVTYSALH KRQVGDYENV IPDFPEDEGI HYSELIQFGV GERPQAQENV |
| 841 | DYVILKH |

CD33 is a type I transmembrane sialoglycoprotein, also known as sialic acid binding immunoglobulin-type lectins (Siglecs). Human CD33 is expressed on hematopoietic and phagocytic cells, namely hematopoietic progenitors, myelomonocytic precursors, macrophages, monocytes, dendritic cells, and microglial cells. Human CD33 preferentially binds to N-acetylneuraminic acid α (2-6) galactose (NeuAc-α (2-6) Gal). Upon ligand binding, the ITIM motif of human CD33 is phosphorylated and recruit SHP phosphatase. This subsequently downregulated activating signaling pathway initiated by ITAM-containing receptors. CD33 has been identified as an AD-associated gene: SNP rs3865444C is an AD-risk allele that promotes the expression of CD33 in microglia and limits Aβ phagocytosis; whereas SNP rs3865444A is an AD protective allele that causes the production of a truncated CD33 lacking a sialic acid binding domain (D2). The resulting truncated CD33 is not expressed on membrane surface and resides in the cytoplasm. Additional information about human CD33, including its exemplary amino acid sequences can be found in public database such as UniProt (UniProt KB/Swiss-Prot ID: P20138) and GENBANK (NCBI Ref. NP_001076087.1, NP_001171079.1, NP_001763.3). An exemplary sequence is also provided below.

| (Seg No.115) | |
|---|---|
| 1 | MPLLLLLPLL WAGALAMDPN FWLQVQESVT VQEGLCVLVP CTFFHPIPYY DKNSPVHGYW |
| 61 | FREGAIISRD SPVATNKLDQ EVQEETQGRF RLLGDPSRNN CSLSIVDARR RDNGSYFFRM |
| 121 | ERGSTKYSYK SPQLSVHVTD LTHRPKILIP GTLEPGHSKN LTCSVSWACE QGTPPIFSWL |
| 181 | SAAPTSLGPR TTHSSVLIIT PRPQDHGTNL TCQVKFAGAG VITERTIQLN VTYVPQNPTT |
| 241 | GIFPGDGSGK QETRAGVVHG AIGGAGVTAL LALCLCLIFF IVKTHRRKAA RTAVGRNDTH |
| 301 | PTTGSASPKH QKKSKLHGPT ETSSCSGAAP TVEMDEELHY ASLNFHGMNP SKDTSTEYSE |
| 361 | VRTQ |

Human CD74 is a type II single spanning transmembrane protein with 4 isoforms. Expression of CD74 is found in lymphocytic and myeloid lineage, such as B cells, T cells, monocyte, macrophage, and dendritic cells. CD74 serves as chaperon of MHCII protein and regulates antigen presentation, endocytic membrane trafficking dendritic cell migration and macrophage migration inhibitory factor (MIF) signaling cascade. CD74 is also expressed in human microglia and is upregulated in neurofibrillar y tangles in Alzheimer's disease. CD74 has been shown to interact with amyloid precursor protein and suppress the production of Aβ. Additional information about human CD74, including its exemplary amino acid sequences can be found in public database such as UniProt (UniProt KB/Swiss-Prot ID: P04233) and GENBANK (NCBI Ref. NP_001020329.1, NP_001020330.1, NP_001351012.1, NP_001351013.1, NP_004346.1). An exemplary sequence is also provided below.

| (Seq No. 116) | |
|---|---|
| 1 | MHRRRSRSCR EDQKPVMDEQ RDLISNNEQL PMLGRRPGAP ESKCSRGALY TGFSILVTLL |
| 61 | LAGQATTAYF LYQQQGRLCK LTVTSQNLQL ENLRMKLPKP PKPVSKMRMA TPLLMQALPM |
| 121 | GALPQGPMQN ATKYGNMTED HVMHLLQNAD PLKVYPPLKG SFPENLRHLK NTMETIDWKV |
| 181 | FESWMHHWLL FEMSRHSLEQ KPTDAPPKES LELEDPSSGL GVTKQDLGPV PM |

CD22 is moderately expressed on microglia cells, which are the major antigen presenting cells in the CNS. The presented antigen (e.g., Aβ or other self-antigens) can be recognized by infiltrating regulatory T cells which dampen the activation of microglia via secretion of IL-10. It was discovered by the inventors of previous disclosure that certain anti-CD22 antibodies can induce internalization of CD22 in human microglia. It was also found that CD22 binds to oligomeric Aβ1-42, and that the internalization of CD22 promotes clearance of Aβ, via both phagocytosis of CD22-bound Aβ1-42 as well as macropinocytosis of soluble Aβ.

Antibodies specific for CD22, CD33 and CD74 were found to induce rapid internalization of the antigen bound antibodies. In the present disclosure, the inventors take advantage of the presence of these internalizing antigens on microglia, astrocyte and oligodendrocyte and present the invention of employing bispecific antibody that specifically bind to CD22, CD33 or CD74 on one end and Aβ on the other end for enhanced elimination of amyloid plaque. Accordingly, in some embodiments, provided herein are methods of promoting removal of Aβ using bispecific antibodies targeting CD22, CD33 or CD74 and Aβ disclosed herein. In some embodiments, provided herein are methods of treating an Aβ-related disease or disorder using bispecific antibodies disclosed herein. In some embodiments, the Aβ-related disease or disorder disease is AD.

As used herein and understood in the art, the “internalization” of CD22, CD33 or CD74 refers to the endocytosis of CD22, CD33 or CD 74 which is the process by which a cell invaginates and engulfs CD22, CD33 or CD74 molecules. There are three types of endocytic process: pinocytosis, phagocytosis and receptor-mediated endocytosis. Pinocytosis is the process by which the cell engulfs small quantities of extracellular fluid (along with small particles that might be present) into the cell via the process of invagination, which is also referred to as “cell-drinking.” Phagocytosis is the process by which a relatively large molecule or organism (such as a bacterial cell) is engulfed by the cell. Receptor mediated endocytosis is the process by which the material binds directly onto the receptor protein of the cell-membrane, which initiates the process of invagination as well as the formation of a protein-covering layer known as the clathrin-coat. The vesicle that is formed in receptor mediated endocytosis contains this clathrin protein coating. CD22, CD33 or CD74 on the cell membrane is internalized upon binding to the specific antibodies via clathrin-mediated endocytosis.

Current anti-Aβ antibody therapies for AD largely rely on FcγR-mediated phagocytosis to reduce amyloid burden, which are associated with the undesired side effects. Specifically, magnetic resonance imaging (MRI) abnormalities, such as signal changes representing “vasogenic edema” (VE) and microhemorrhages (mH) have been observed in patients administered with amyloid-modifying therapeutic agents (anti-Aβ antibody) in a dose dependent manner. Amyloid-related imaging abnormalities (ARIA) is suggested by the Alzheimer's Association Rescarch Roundtable Workgroup to describe vasogenic edema (ARIA-E) and microhemorrhages and hemosiderosis (ARIA-H). ARIA-E is largely caused by inflammation and tightly coupled to mAb that binds to fibril form of Aβ. The FcγR-mediated inflammation in microglia can take a major role. For example, the inflammation mediated by FcγR-induced proinflammatory cytokine releases exacerbate damage to vascular integrity. Crenzumab, a humanized IgG4 antibody targeting all form of Aβ is associated with less ARIA-E events, as the IgG4 Fc region has reduced association to FcγR of microglia as compared to, for example, an IgG1 Fc.

As such, bispecific antibodies targeting CD22, CD33 or CD47 on one end and Aβ on the other end provided herein provide a novel mechanism of Aβ clearance that is not only highly efficient, but also associated with reduced vascular side effect like ARIA-E and/or ARIA-H. Unlike the Aβ-targeting antibodies, the bispecific antibodies disclosed herein promotes Aβ clearance via CD22, CD33 or CD74 internalization, which does not involve the cross-linking of the therapeutic antibodies with FcγR, thereby avoiding the subsequent pro-inflammatory response. Accordingly, provided here are methods of efficient Aβ clearance that have reduced vascular side effect. In some embodiments, methods provided herein do not cause vascular side effect.

The bispecific antibodies targeting CD22 and Aβ (e.g., SM03 or SM06) disclosed herein, in addition to promoting Aβ removal via CD22 internalization, (a) promote cis-trans conversion of CD22 and/or (b) induce internalization of CD22. The bispecific antibodies targeting CD22 and Aβ disclosed herein promote cis-trans conversion of CD22 by disrupting the cis-binding of CD22 on the surface of B-cells, microglia cells or oligodendrocyte as well as accelerating the internalization of surface CD22. When the CD22 molecules are internalized with antibodies that disrupt the cis-binding of the homo-cluster, the CD22 molecules are recycled to the cell surface with the antibodies, where the antibodies sterically hinder further cis-binding, thereby promoting trans-binding of the recycled CD22. As such, the bispecific antibodies targeting CD22 and Aβ disclosed herein can also promote immune-tolerance in both central and peripheral immune system by disrupting cis-binding of CD22 on B cell, B cell-derived cell lines and microglia, and promoting cis-trans conversion of 2,6-sialic acid binding of self-tissue, which allows regaining immune tolerance of self-tissue and dampens pro-inflammatory cytokine release. By promoting trans-binding of CD22 to neuronal 2,6-sialic acid, the anti-CD22 antibodies disclosed herein inhibit phagocytosis of synapse and neuronal death, as well as suppress NF-κB signaling and interleukin-6 (IL-6) secretion in microglia cells. Thus, methods disclosed herein have the additional therapeutic benefit of reducing neuroinflammation. Accordingly, in some embodiments, provided herein are methods of reducing neuroinflammation using anti-CD22 antibodies disclosed herein. In some embodiments, provided herein are methods of treating a neuroinflammation-associated disease or disorder using anti-CD22 antibodies disclosed herein.

The bispecific antibodies targeting CD33 and Aβ (e.g., Mylot-Aβ, 96-Aβ or Lin-Aβ) disclosed herein induce internalization of CD33 and induce SHP-1 phosphorylation and subsequent inhibition of microglial activation.

The bispecific antibodies targeting CD74 and Aβ (e.g., LL1-Aβ) disclosed herein induce internalization of CD74 and block MIF signaling cascade. This inhibitory effect on MIF reduces neuroinflammation in microglia. Anti-CD74 antibody disclosed herein increased the interaction of CD74 and APP and reduced the production of Aβ.

Provided herein are methods of promoting removal of Aβ plaque in a subject in need thereof that comprise administering to the subject a therapeutically effective amount of a bispecific antibody or antigen-binding fragment thereof that specifically binds to CD22, CD33 or CD74 on one end and Aβ on the other end wherein the bispecific antibody or antigen-binding fragment (a) induces internalization of the surface receptors/antigens and/or (b) suppresses microglia, astrocyte, oligodendrocyte, pericyte brain endothelial cell or other neuro-immunological cell activation which promote neuro-inflammation and/or (c) reduces pathogenic synaptic pruning by microglia, astrocyte or other neuro-immunological cell and/or (d) modulate antigen presentation by microglia, astrocyte, endothelial cell, or other neuro-immunological cell to infiltrating T cells and/or (c) promote remyelination of damaged myelin sheath by oligodendrocyte or other neuro-immunological cells. Provided herein are also methods of treating an Aβ-related disease or disorder in a subject in need thereof that comprise administering to the subject a therapeutically effective amount of an antibody or antigen-binding fragment thereof that specifically binds to CD22, CD33 or CD74 wherein the antibody or antigen-binding fragment (a) induces internalization of the surface receptors/antigens and/or (b) suppresses microglia, astrocyte, oligodendrocyte, pericyte brain endothelial cell or other neuro-immunological cell activation which promote neuro-inflammation and/or (c) reduces pathogenic synaptic pruning by microglia, astrocyte or other neuro-immunological cell and/or (d) modulate antigen presentation by microglia, astrocyte, endothelial cell, or other neuro-immunological cell to infiltrating T cells and/or (c) promote remyclination of damaged myelin sheath by oligodendrocyte or other neuro-immunological cells. In some embodiments, the subject is a human. In some embodiments, the antibody or antigen-binding fragment thereof specifically binds to human CD22, CD33 or CD74. In some embodiments, the Aβ-related disease or disorder is AD. In some embodiments, the Aβ-related disease or disorder is preclinical AD.

In some embodiments, provided herein are uses of a bispecific antibody or antigen-binding fragment thereof that specifically binds to CD22, CD33 or CD47 on one end and Aβ on the other end for removing Aβ plaque. In some embodiments, provided herein are uses of a bispecific antibody or antigen-binding fragment thereof that specifically binds to CD22, CD33 or CD74 on one end and Aβ on the other end for the preparation of a medicament for the removal of Aβ plaque. In some embodiments, provided herein are uses of a bispecific antibody or antigen-binding fragment thereof that specifically binds to CD22, CD33 or CD74 on one end and Aβ on the other end in the treatment of an Aβ-related disease or disorder. In some embodiments, provided herein are uses of a bispecific antibody or antigen-binding fragment thereof that specifically binds to CD22, CD33 or CD74 on one end and Aβ on the other end for the preparation of a medicament for the treatment of an Aβ-related disease or disorder. In the uses described above, the bispecific antibody or antigen-binding fragment (a) induces internalization of the surface receptors/antigens and/or (b) suppresses microglia, astrocyte, oligodendrocyte, pericyte brain endothelial cell or other neuro-immunological cell activation which promote neuro-inflammation and/or (c) reduces pathogenic synaptic pruning by microglia, astrocyte or other neuro-immunological cell and/or (d) modulate antigen presentation by microglia, astrocyte, endothelial cell, or other neuro-immunological cell to infiltrating T cells and/or (c) promote remyelination of damaged myelin sheath by oligodendrocyte or other neuro-immunological cells. In some embodiments, the Aβ-related disease or disorder is AD. In some embodiments, the Aβ-related disease or disorder is preclinical AD. In some embodiments, the antibody or antigen-binding fragment thereof specifically binds to human CD22, CD33 or CD74.

With the treatment of the CD22, CD33 or CD74 bispecific antibodies disclosed herein, the rate of internalization of Aβ can reach 5.86×10⁶ pg/s/cell, as measured using ELISA of Aβ-treated microglia cell lysate (data not shown). In some embodiments of the uses or methods provided herein, Aβ is removed at a rate of 0.86 pg/s.

Methods provided herein can treat an Aβ-related disease or disorder. The Aβ-related disease or disorder can be clinical or pre-clinical AD, prodromal AD, Down's syndrome, clinical or pre-clinical amyloid angiopathy (CAA), Parkinson's disease, multi-infarct dementia, cerebral amyloid angiopathy, glaucoma, pre-eclampsia, cognitive impairment, memory loss, or a vascular disorder caused by pathogenic Aβ peptide in blood vessel.

In some embodiments, methods provided herein can be used to treat AD. As described above and well known in the art, removal of oligomeric Aβ is known to provide clinical benefits in AD. The AD can be clinical AD, pre-clinical AD or prodromal AD. In some embodiments, methods provided herein can be used to treat clinical AD. In some embodiments, methods provided herein can be used to treat pre-clinical AD. In some embodiments, methods provided herein can be used to treat prodromal AD.

In some embodiments, methods provided herein can be used to treat Down's syndrome. In some embodiments, methods provided herein can be used to treat clinical or pre-clinical CAA. In some embodiments, methods provided herein can be used to treat Parkinson's disease. In some embodiments, methods provided herein can be used to treat multi-infarct dementia. In some embodiments, methods provided herein can be used to treat cerebral amyloid angiopathy. In some embodiments, methods provided herein can be used to treat glaucoma. In some embodiments, methods provided herein can be used to treat pre-eclampsia. In some embodiments, methods provided herein can be used to treat cognitive impairment. In some embodiments, methods provided herein can be used to treat memory loss. In some embodiments, methods provided herein can be used to treat a vascular disorder caused by pathogenic Aβ peptide in blood vessel. Provided herein are also methods of reducing neuroinflammation in a subject in need thereof that comprise administering to the subject a therapeutically effective amount of a bispecific antibody or antigen-binding fragment thereof that, in addition to binding to Aβ protein, specifically binds to CD22, CD33 or CD74, wherein the bispecific antibody or antigen-binding fragment (a) induces internalization of the surface receptors/antigens and/or (b) suppresses microglia, astrocyte, oligodendrocyte, pericyte brain endothelial cell or other neuro-immunological cell activation which promote neuro-inflammation and/or (c) reduces pathogenic synaptic pruning by microglia, astrocyte or other neuro-immunological cell and/or (d) modulate antigen presentation by microglia, astrocyte, endothelial cell, or other neuro-immunological cell to infiltrating T cells and/or (e) promote remyelination of damaged myelin sheath by oligodendrocyte or other neuro-immunological cells. Provided herein are also methods of treating a disease or disorder associated with neuroinflammation in a subject in need thereof that comprise administering to the subject a therapeutically effective amount of a bispecific antibody or antigen-binding fragment thereof that the receptor-binding portion specifically binds to CD22, CD33 or CD74, wherein the bispecific antibody or antigen-binding fragment (a) induces internalization of the surface receptors/antigens and/or (b) suppresses microglia, astrocyte, oligodendrocyte, pericyte brain endothelial cell or other neuro-immunological cell activation which promote neuro-inflammation and/or (c) reduces pathogenic synaptic pruning by microglia, astrocyte or other neuro-immunological cell and/or (d) modulate antigen presentation by microglia, astrocyte, endothelial cell, or other neuro-immunological cell to infiltrating T cells and/or (e) promote remyelination of damaged myelin sheath by oligodendrocyte or other neuro-immunological cells. In some embodiments, the methods provided herein prevent synaptic phagocytosis and neuronal death. In some embodiments, the methods provided herein prevent synaptic phagocytosis and neuronal death by at least 20%, at least 30, at least 40%, at least over 50%, or at least over 60%. In some embodiments, the methods provided herein prevent synaptic phagocytosis and neuronal death by at least 50%. In some embodiments, the subject is a human. In some embodiments, the antibody or antigen-binding fragment thereof specifically binds to human CD22, CD33 or CD74.

In some embodiments, provided herein are uses of a bispecific antibody or antigen-binding fragment thereof that specifically binds to CD22, CD33 or CD74 for reducing neuroinflammation. In some embodiments, provided herein are uses of a bispecific antibody or antigen-binding fragment thereof that specifically binds to CD22, CD33 or CD74 for the preparation of a medicament for the reduction of neuroinflammation. In some embodiments, provided herein are uses of a bispecific antibody or antigen-binding fragment thereof that specifically binds to CD22, CD33 or CD74 in the treatment of a disease or disorder associated with neuroinflammation. In some embodiments, provided herein are uses of a bispecific antibody or antigen-binding fragment thereof that specifically binds to CD22, CD33 or CD74 for the preparation of a medicament for the treatment of a disease or disorder associated with neuroinflammation. In the uses described above, the bispecific antibody or antigen-binding fragment (a) induces internalization of the surface receptors/antigens and/or (b) suppresses microglia, astrocyte, oligodendrocyte, pericyte brain endothelial cell or other neuro-immunological cell activation which promote neuro-inflammation and/or (c) reduces pathogenic synaptic pruning by microglia, astrocyte or other neuro-immunological cell and/or (d) modulate antigen presentation by microglia, astrocyte, endothelial cell, or other neuro-immunological cell to infiltrating T cells and/or (c) promote remyelination of damaged myelin sheath by oligodendrocyte or other neuro-immunological cells. In some embodiments, the antibody or antigen-binding fragment thereof specifically binds to human CD22, CD33 or CD74.

In some embodiments, the anti-CD22, CD33 or CD74 bispecific antibody or antigen-binding fragment used in the methods described herein induces internalization of CD22, CD33 or CD74. In some embodiments, the anti-CD22 bispecific antibody or antigen-binding fragment, and possibly the anti-CD33 bispecific antibody or antigen-binding fragment used in the methods described herein promote cis-trans conversion of CD22 and CD33. In some embodiments, the anti-CD22 and anti-CD33 bispecific antibodies or antigen-binding fragments used in the methods described herein promote cis-trans conversion and induce internalization of CD22 and CD33.

As used herein, the term “treat” and its grammatical equivalents in connection with a disease or a condition, or a subject having a disease or a condition refer to an action that suppresses, eliminates, reduces, and/or ameliorates a symptom, the severity of the symptom, and/or the frequency of the symptom associated with the disease or disorder being treated. For example, when used in reference to AD, the term “treat” and its grammatical equivalents refer to an action that reduces the severity of the disease, or retards or slows the progression of the disease, including, but not limited to (a) reducing the amount of Aβ plaque, or slowing the rate of accumulation of pathologic AB, or (b) delaying, ameliorating or minimizing one or more symptoms associated with AD, such as dementia or cognitive impairment.

As used herein, the term “administer” and its grammatical equivalents refer to the act of delivering, or causing to be delivered, a therapeutic or a pharmaceutical composition to the body of a subject by a method described herein or otherwise known in the art. The therapeutic can be a compound, a polypeptide, or a cell. Administering a therapeutic or a pharmaceutical composition includes prescribing a therapeutic or a pharmaceutical composition to be delivered into the body of a subject. Exemplary forms of administration include oral dosage forms, such as tablets, capsules, syrups, suspensions; injectable dosage forms, such as intravenous (IV), intramuscular (IM), or intraperitoneal (IP); subcutaneous (SC), transdermal dosage forms, including creams, jellies, powders, or patches; buccal dosage forms; inhalation powders, sprays, suspensions, and rectal suppositories.

As used herein, the terms “effective amount,” “therapeutically effective amount,” and their grammatical equivalents refer to the administration of an agent to a subject, either alone or as a part of a pharmaceutical composition and either in a single dose or as part of a series of doses, in an amount that is capable of having any detectable, positive effect on any symptom, aspect, or characteristics of a disease, disorder or condition when administered to the subject. The therapeutically effective amount can be ascertained by measuring relevant physiological effects. The exact amount required vary from subject to subject, depending on the age, weight, and general condition of the subject, the severity of the condition being treated, the judgment of the clinician, and the like. A therapeutically effective amount is also one in which any toxic or detrimental effects of the therapeutic agent are outweighed by the therapeutically beneficial effects. An appropriate “effective amount” in any individual case can vary according to factors such as the disease state, age, sex, and weight of the individual, and can be determined by one of ordinary skill in the art using routine experimentation. A “prophylactically effective amount” refers to an amount effective, at dosages and for periods of time necessary, to achieve the desired prophylactic result, for example, the delay or prevention of the onset of a disease or disorder. Typically, since a prophylactic dose is used in subjects prior to or at an earlier stage of disease, the prophylactically effective amount is commonly less than the therapeutically effective amount.

The term “subject” as used herein refers to any animal (e.g., a mammal), including, but not limited to, humans, non-human primates, canines, felines, rodents, and the like, which is to be the recipient of a particular treatment. A subject can be a human. A subject can be a patient with a particular disease.

### 7.2 Antibodies Specific for Internalizing Receptors Expressed on Microglia Cells, Oligodendrocyte, Astrocyte.

As described in the section above, provided herein are methods and uses of bispecific antibodies or antigen-binding fragment thereof, that, in addition to binding to Aβ protein, specifically bind to internalizing receptors and/or antigens expressed on microglia cells such as CD22, CD33 and CD74. The term “antibody,” and its grammatical equivalents as used herein refer to an immunoglobulin molecule that recognizes and specifically binds a target, such as a protein, polypeptide, peptide, carbohydrate, polynucleotide, lipid, or a combination of any of the foregoing, through at least one antigen-binding site wherein the antigen-binding site is usually within the variable region of the immunoglobulin molecule. As used herein, the term encompasses intact polyclonal antibodies, intact monoclonal antibodies, single-domain antibodies (sdAbs; e.g., camelid antibodies, alpaca antibodies), single-chain Fv (scFv) antibodies, heavy chain antibodies (HCAbs), light chain antibodies (LCAbs), multispecific antibodies, bispecific antibodies, monospecific antibodies, monovalent antibodies, and any other modified immunoglobulin molecule comprising an antigen-binding site (e.g., dual variable domain immunoglobulin molecules) as long as the antibodies exhibit the desired biological activity. Antibodies also include, but are not limited to, mouse antibodies, rabbit antibodies, camel antibodies, primate antibodies, chimeric antibodies, humanized antibodies, and human antibodies. An antibody can be any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. In some embodiments, an antibody can comprise four polypeptide chains, two heavy (H) chains and two light (L) chains interconnected by disulfide bonds. Each heavy chain is comprised of a heavy chain variable region (abbreviated herein as HCVR or VH) and a heavy chain constant region. Each light chain is comprised of a light chain variable region (abbreviated herein as LCVR or VL) and a light chain constant region.

Unless expressly indicated otherwise, the term “antibody” as used herein include “antigen-binding fragment” of intact antibodies. The term “antigen-binding fragment” as used herein refers to a portion or fragment of an intact antibody that is the antigenic determining variable region of an intact antibody. Examples of antigen-binding fragments include, but are not limited to, Fab (a monovalent fragment consisting of the VL, VH, CL and CHI domains without the hinge region), Fab′ (amonovalent fragment consisting of the VL, VH, CL and CHI domains attached with a hinge region), F(ab′) 2 (a bivalent fragment comprising two Fab fragments linked by a disulfide bridge at the hinge region), Fd (a fragment consisting of the VH and CHI domains), Fv (a fragment consisting of the VL and VH domains of a single arm of an antibody), linear antibodies, single chain antibody molecules (e.g., scFv, which is a single polypeptide chain having VL and VH regions joined by recombinant means), heavy chain antibodies (HCAbs), light chain antibodies (LCAbs), disulfide-linked scFv (dsscFv), diabodies (bivalent, bispecific antibodies), tribodies, tetrabodies, minibodies, dual variable domain antibodies (DVD), single variable domain antibodies (sdAbs or dAbs; e.g., camelid antibodies, alpaca antibodies), and single variable domain of heavy chain antibodies (VHH), and bispecific or multispecific antibodies formed from antibody fragments. A “bispecific” antibody is an artificial hybrid antibody having two different antigen binding sites, which recognize and specifically bind two different targets. Bispecific antibodies can be produced by a variety of methods including fusion of hybridomas or linking of Fab′ fragments. See, e.g., Songsivilai &Lachmann, Clin. Exp. Immunol. 79:315-321 (1990); Kostelny et al., J. Immunol. 148, 1547-1553 (1992); Labrijn A F, et. al. Bispecific antibodies: a mechanistic review of the pipeline. Nat Rev Drug Discov. 18 (8): 585-608 (2019); Nic S et. al. Biology drives the discovery of bispecific antibodies as innovative therapeutics. Antib Ther. 17; 3 (1): 18-62 (2020).

An antibody or antigen-binding fragment thereof can be part of a larger immunoadhesion molecules, formed by covalent or noncovalent association of the antibody or antigen-binding fragment with one or more other proteins or peptides. Examples of such immunoadhesion molecules include use of the streptavidin core region to make a tetrameric scFv molecule (Kipriyanov et al. (1995) Human antibodies and Hybridomas 6:93-101) and use of a cysteine residue, a marker peptide and a C-terminal polyhistidine tag to make bivalent and biotinylated scFv molecules (Kipriyanov et al. (1994) Mol. Immunol. 31:1047-1058). Antigen-binding fragments, such as Fab, Fab′ and F(ab′) 2 fragments, can be prepared from whole antibodies using conventional techniques, such as papain or pepsin digestion, respectively, of whole antibodies. Moreover, antibodies, antigen-binding fragments and immunoadhession molecules can be obtained using standard recombinant DNA techniques, as described herein.

The term “humanized antibody” as used herein refers to forms of non-human (e.g., murine) antibodies that are specific immunoglobulin chains, chimeric immunoglobulins, or fragments thereof that contain minimal non-human sequences. Typically, humanized antibodies are human immunoglobulin. In some instances, the Fv framework region residues of a human immunoglobulin are replaced with the corresponding residues in an antibody from a non-human species. In some instances, residues of the CDRs are replaced by residues from the CDRs of a non-human species (e.g., mouse, rat, hamster, camel) that have the desired specificity, affinity, and/or binding capability. The humanized antibody can be further modified by the substitution of additional residues either in the Fv framework region and/or within the replaced non-human residues to refine and optimize antibody specificity, affinity, and/or binding capability. The term “human antibody” as used herein refers to an antibody produced by a human or an antibody having an amino acid sequence corresponding to an antibody produced by a human made using any of the techniques known in the art.

The term “heavy chain” when used in reference to an antibody refers to a polypeptide chain of about 50-70 kDa, wherein the amino-terminal portion includes a variable region (VH) of about 120 to 130 or more amino acids and a carboxy-terminal portion that includes a constant region. In some embodiments, the heavy chain constant region is comprised of three domains, CH1, CH2 and CH3, and there is a short flexible hinge region connecting the CH1 and CH2 domains. The constant region can be one of five distinct types, referred to as alpha (α), delta (δ), epsilon (ε), gamma (γ) and mu (μ), based on the amino acid sequence of the heavy chain constant region. The distinct heavy chains differ in size: α, δ and γ contain approximately 450 amino acids, while u and ε contain approximately 550 amino acids. When combined with a light chain, these distinct types of heavy chains give rise to five well known classes of antibodies, IgA, IgD, IgE, IgG and IgM, respectively, including four subclasses of IgG, namely IgG1, IgG2, IgG3 and IgG4. A heavy chain can be a human heavy chain.

The term “light chain” when used in reference to an antibody refers to a polypeptide chain of about 25 kDa, wherein the amino-terminal portion includes a variable region of about 100 to about 110 or more amino acids and a carboxy-terminal portion that includes a constant region. The light chain constant region is comprised of one domain, CL. The approximate length of a light chain is 211 to 217 amino acids. There are two distinct types, referred to as kappa (κ) of lambda (λ) based on the amino acid sequence of the constant domains. Light chain amino acid sequences are well known in the art. A light chain can be a human light chain.

The term “variable domain” or “variable region” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable domains differ extensively in sequence between different antibodies. The variability in sequence is concentrated in the CDRs while the less variable portions in the variable domain are referred to as framework regions (FR). The CDRs of the light and heavy chains are primarily responsible for the interaction of the antibody with antigen. In some embodiments, each VH and VL is composed of three CDRs and four FRs, arranged from amino-terminus to carboxy-terminus in the following order: FR1, CDR1, FR2, CDR2, FR3, CDR3, FR4. Numbering of amino acid positions used herein is according to the EU Index, as in Kabat et al. (1991) Sequences of proteins of immunological interest. (U.S. Department of Health and Human Services, Washington, D.C.) 5thed.

A CDR refers to one of three hypervariable regions (H1, H2 or H3) within the non-framework region of the immunoglobulin (Ig or antibody) VH β-sheet framework, or one of three hypervariable regions (L1, L2 or L3) within the non-framework region of the antibody VL β-sheet framework. Accordingly, CDRs are variable region sequences interspersed within the framework region sequences. CDR regions are well known to those skilled in the art and have been defined by a variety of methods/systems. These systems and/or definitions have been developed and refined over years and include Kabat, Chothia, IMGT, AbM, and Contact. For example, Kabat defines the regions of most hypervariability within the antibody variable (V) domains (Kabat et al, J. Biol. Chem. 252:6609-6616 (1977); Kabat, Adv. Prot. Chem. 32:1-75 (1978)). The Chothia definition is based on the location of the structural loop regions, which defines CDR region sequences as those residues that are not part of the conserved β-sheet framework, and thus are able to adapt different conformations (Chothia and Lesk, J. Mol. Biol. 196:901-917 (1987)). Both terminologies are well recognized in the art. Additionally, the IMGT system is based on sequence variability and location within the structure of the variable regions. The AbM definition is a compromise between Kabat and Chothia. The Contact definition is based on analyses of the available antibody crystal structures. Software programs (e.g., abYsis) are available and known to those of skill in the art for analysis of antibody sequence and determination of CDRs. The positions of CDRs within a canonical antibody variable domain have been determined by comparison of numerous structures (Al-Lazikani et al, J. Mol. Biol. 273:927-948 (1997); Morea et al, Methods 20:267-279 (2000)). Because the number of residues within a hypervariable region varies in different antibodies, additional residues relative to the canonical positions are conventionally numbered with a, b, c and so forth next to the residue number in the canonical variable domain numbering scheme (Al-Lazikani et al., supra (1997)). Such nomenclature is similarly well known to those skilled in the art.

For example, CDRs defined according to either the Kabat (hypervariable) or Chothia (structural) designations, are set forth in the table below.

| | Kabat¹ | Chothia² | Loop Location |
|---|---|---|---|
| VHCDR1 | 31-35 | 26-32 | linking B and C strands |
| VHCDR2 | 50-65 | 53-55 | linking C′ and C″ strands |
| VHCDR3 | 95-102 | 96-101 | linking F and G strands |
| VLCDR1 | 24-34 | 26-32 | linking B and C strands |
| VLCDR2 | 50-56 | 50-52 | linking C′ and C″ strands |
| VLCDR3 | 89-97 | 91-96 | linking F and G strands |

¹Residue numbering follows the nomenclature of Kabat et al., supra
²Residue numbering follows the nomenclature of Chothia et al., supra

One or more CDRs also can be incorporated into a molecule either covalently or noncovalently to make it an immunoadhesin. An immunoadhesin can incorporate the CDR(s) as part of a larger polypeptide chain, can covalently link the CDR(s) to another polypeptide chain, or can incorporate the CDR(s) noncovalently. The CDRs permit the immunoadhesin to bind to a particular antigen of interest.

The terms “epitope” and “antigenic determinant” are used interchangeably herein an refer to the site on the surface of a target molecule to which an antibody or antigen-binding fragment binds, such as a localized region on the surface of an antigen. The target molecule can comprise, a protein, a peptide, a nucleic acid, a carbohydrate, or a lipid. An epitope having immunogenic activity is a portion of a target molecule that elicits an immune response in an animal. An epitope of a target molecule having antigenic activity is a portion of the target molecule to which an antibody binds, as determined by any method well known in the art, including, for example, by an immunoassay. Antigenic epitopes need not necessarily be immunogenic. Epitopes often consist of chemically active surface groupings of molecules such as amino acids or sugar side chains and have specific three dimensional structural characteristics as well as specific charge characteristics. The term, “epitope” includes linear epitopes and conformational epitopes. A region of a target molecule (e.g., a polypeptide) contributing to an epitope can be contiguous amino acids of the polypeptide or the epitope can come together from two or more non-contiguous regions of the target molecule. The epitope may or may not be a three-dimensional surface feature of the target molecule. Epitopes formed from contiguous amino acids (also referred to as linear epitopes) are typically retained upon protein denaturing, whereas epitopes formed by tertiary folding (also referred to as conformational epitopes) are typically lost upon protein denaturing. An epitope typically includes at least 3, and more usually, at least 5, 6, 7, or 8-10 amino acids in a unique spatial conformation.

The term “specifically binds,” as used herein, means that a polypeptide or molecule interacts more frequently, more rapidly, with greater duration, with greater affinity, or with some combination of the above to the epitope, protein, or target molecule than with alternative substances, including related and unrelated proteins. A binding moiety (e.g., antibody) that specifically binds a target molecule (e.g., antigen) can be identified, for example, by immunoassays, ELISAs, Bio-Layer Interferometry (“BLI”), SPR (e.g., Biacore), or other techniques known to those of skill in the art. Typically, a specific reaction will be at least twice background signal or noise and can be more than 10 times background. Sec, e.g., Paul, ed., 1989, FUNDAMENTAL IMMUNOLOGY SECOND EDITION, Raven Press, New York at pages 332-336 for a discussion regarding antibody specificity. A binding moiety that specifically binds a target molecule can bind the target molecule at a higher affinity than its affinity for a different molecule. In some embodiments, a binding moiety that specifically binds a target molecule can bind the target molecule with an affinity that is at least 20 times greater, at least 30 times greater, at least 40 times greater, at least 50 times greater, at least 60 times greater, at least 70 times greater, at least 80 times greater, at least 90 times greater, or at least 100 times greater, than its affinity for a different molecule. In some embodiments, a binding moiety that specifically binds a particular target molecule binds a different molecule at such a low affinity that binding cannot be detected using an assay described herein or otherwise known in the art. In some embodiments, “specifically binds” means, for instance, that a binding moiety binds a molecule target with a K_{D} of about 0.1 mM or less. In some embodiments, “specifically binds” means that a polypeptide or molecule binds a target with a K_{D} of at about 10 μM or less or about 1 μM or less. In some embodiments, “specifically binds” means that a polypeptide or molecule binds a target with a K_{D} of at about 0.1 μM or less, about 0.01 μM or less, or about 1 nM or less. Because of the sequence identity between homologous proteins in different species, specific binding can include a polypeptide or molecule that recognizes a protein or target in more than one species. Likewise, because of homology within certain regions of polypeptide sequences of different proteins, specific binding can include a polypeptide or molecule that recognizes more than one protein or target. It is understood that, in some embodiments, a binding moiety (e.g., antibody) that specifically binds a first target may or may not specifically bind a second target. As such, “specific binding” does not necessarily require (although it can include) exclusive binding, i.e., binding to a single target. Thus, a binding moiety (e.g., antibody) can, in some embodiments, specifically bind more than one target. For example, an antibody can, in certain instances, comprise two identical antigen-binding sites, each of which specifically binds the same epitope on two or more proteins. In certain alternative embodiments, an antibody can be bispecific and comprise at least two antigen-binding sites with differing specificities.

The term “binding affinity” as used herein generally refers to the strength of the sum total of noncovalent interactions between a binding moiety and a target molecule (e.g., antigen). The binding of a binding moiety and a target molecule is a reversible process, and the affinity of the binding is typically reported as an equilibrium dissociation constant (K_{D}). K_{D} is the ratio of a dissociation rate (k_{off} or k_{d}) to the association rate (kₒₙ or kₐ). The lower the K_{D} of a binding pair, the higher the affinity. K_{A} is the equilibrium association constant, which is also the reciprocal of the equilibrium dissociation constant, i.e., =1/K_{D}. For an antibody-antigen interaction, K_{D} can be calculated as the ratio of the products of concentrations of free antibody and free antigen over the concentrations of antibody-antigen complex, i.e., [antigen]×[antibody]/[antigen−antibody].

A variety of methods of measuring binding affinity are known in the art, any of which can be used for purposes of the present disclosure. Specific illustrative embodiments include the following. In some embodiments, the “K_{D}” or “K_{D} value” can be measured by assays known in the art, for example by a binding assay. The K_{D} may be measured in a radiolabeled antigen binding assay (RIA) (Chen, et al., (1999) J. Mol Biol 293:865-881). The K_{D} or K_{D} value can also be measured by using biolayer interferometry (BLI) using, for example, the Gator system (Probe Life), or the Octet-96 system (Sartorius AG). The K_{D} or K_{D} value can also be measured by using surface plasmon resonance assays by using a BIAcore system (e.g., Pharmacia Biosensor Aβ, Uppsala, Sweden and Piscataway, NJ).

The term “variant” as used herein in relation to a protein or a polypeptide with particular sequence features (the “reference protein” or “reference polypeptide”) refers to a different protein or polypeptide having one or more (such as, for example, about 1 to about 25, about 1 to about 20, about 1 to about 15, about 1 to about 10, or about 1 to about 5) amino acid substitutions, deletions, and/or additions as compared to the reference protein or reference polypeptide. The changes to an amino acid sequence can be amino acid substitutions. The changes to an amino acid sequence can be conservative amino acid substitutions. A functional fragment or a functional variant of a protein or polypeptide maintains the basic structural and functional properties of the reference protein or polypeptide.

The terms “polypeptide,” “peptide,” “protein,” and their grammatical equivalents as used interchangeably herein refer to polymers of amino acids of any length, which can be linear or branched. It can include unnatural or modified amino acids or be interrupted by non-amino acids. A polypeptide, peptide, or protein can also be modified with, for example, disulfide bond formation, glycosylation, lipidation, acetylation, phosphorylation, or any other manipulation or modification.

The terms “polynucleotide,” “nucleic acid,” and their grammatical equivalents as used interchangeably herein mean polymers of nucleotides of any length and include DNA and RNA. The nucleotides can be deoxyribonucleotides, ribonucleotides, modified nucleotides or bases, and/or their analogs, or any substrate that can be incorporated into a polymer by DNA or RNA polymerase. A nucleic acid molecule can be single-stranded or double-stranded.

As used herein, the term “encode” and its grammatical equivalents refer to the inherent property of specific sequences of nucleotides in a polynucleotide or a nucleic acid, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein. Unless otherwise specified, a “nucleotide sequence encoding an amino acid sequence” includes all nucleotide sequences that are degenerate versions of each other and that encode the same amino acid sequence. Nucleotide sequences that encode proteins and RNA can include introns.

A polypeptide, peptide, protein, antibody, polynucleotide, vector, cell, or composition which is “isolated” is a polypeptide, peptide, protein, antibody, polynucleotide, vector, cell, or composition which is in a form not found in nature. Isolated polypeptides, peptides, proteins, antibodies, polynucleotides, vectors, cells, or compositions include those which have been purified to a degree that they are no longer in a form in which they are found in nature. In some embodiments, a polypeptide, peptide, protein, antibody, polynucleotide, vector, cell, or composition which is isolated is substantially pure. In some embodiments, a polypeptide, peptide, protein, antibody, polynucleotide, vector, cell, or composition which is isolated is substantially free of other cellular material and/or chemicals.

The terms “identical,” percent “identity,” and their grammatical equivalents as used herein in the context of two or more polynucleotides or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software that can be used to obtain alignments of amino acid or nucleotide sequences are well-known in the art. These include, but are not limited to, BLAST, ALIGN, Megalign, BestFit, GCG Wisconsin Package, and variants thereof. In some embodiments, two polynucleotides or polypeptides provided herein are substantially identical, meaning they have at least 70%, at least 75%, at least 80%, at least 85%, at least 90%, and in some embodiments at least 95%, at least 96%, at least 97%, at least 98%, or at least 99% nucleotide or amino acid residue identity, when compared and aligned for maximum correspondence, as measured using a sequence comparison algorithm or by visual inspection. In some embodiments, identity exists over a region of the amino acid sequences that is at least about 10 residues, at least about 20 residues, at least about 40-60 residues, at least about 60-80 residues in length or any integral value there between. In some embodiments, identity exists over a longer region than 60-80 residues, such as at least about 80-100 residues, and in some embodiments the sequences are substantially identical over the full length of the sequences being compared, such as the coding region of a target protein or an antibody. In some embodiments, identity exists over a region of the nucleotide sequences that is at least about 10 bases, at least about 20 bases, at least about 40-60 bases, at least about 60-80 bases in length or any integral value there between. In some embodiments, identity exists over a longer region than 60-80 bases, such as at least about 80-1000 bases or more, and in some embodiments the sequences are substantially identical over the full length of the sequences being compared, such as a nucleotide sequence encoding a protein of interest.

A “conservative amino acid substitution” as used herein, is one in which one amino acid residue is replaced with another amino acid residue having a similar side chain. Amino acid or residue that is “conservatively similar” as used herein refers to non-identical amino acid residue having similar side chains. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The term “vector,” and its grammatical equivalents as used herein refer to a vehicle that is used to carry genetic material (e.g., a polynucleotide sequence), which can be introduced into a host cell, where it can be replicated and/or expressed. Vectors applicable for use include, for example, expression vectors, plasmids, phage vectors, viral vectors, episomes and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more polynucleotides are to be co-expressed, both polynucleotides can be inserted, for example, into a single expression vector or in separate expression vectors. For single vector expression, the encoding polynucleotides can be operationally linked to one common expression control sequence or linked to different expression control sequences, such as one inducible promoter and one constitutive promoter. The introduction of polynucleotides into a host cell can be confirmed using methods well known in the art. It is understood by those skilled in the art that the polynucleotides are expressed in a sufficient amount to produce a desired product (e.g., an anti-CD22, anti-CD33 or anti-CD74 bispecific antibody or antigen-binding fragment that also targets Aβ protein), and it is further understood that expression levels can be optimized to obtain sufficient expression using methods well known in the art.

As used herein, the term “host cell” refers to a cell into which a genetical material, such as a recombinant expression vector can be introduced or has been introduced. Host cells include not only the subject cell introduced with the exogenous genetic material, but also the progeny of such a cell. Because certain modifications may occur in succeeding generations due to either mutation or environmental influences, such progeny may not be identical to the parent cell.

As used herein and understood in the art, “EC” means effective concentration of an agent (e.g., antibody), and is commonly used in dose-response curves. The “effect” of the agent can be a positive (activatory) effect or a negative effect. The term “EC50” refers to the concentration of an active agent (e.g., antibody) that gives half-maximal response. Also as used herein and understood in the art, “IC” means concentration of an agent that has an inhibitory effect, and is also commonly used for dose-response curves. The term “IC50” refers to the concentration of an agent (e.g., antibody) where the activity that it inhibits is reduced by half.

### 7.2.1 Exemplary Anti-CD22, Anti-CD33 and Anti-CD74 Bispecific Antibodies

As described in the section above, provided herein are methods and uses of bispecific antibodies or antigen-binding fragment thereof, that, in addition to binding to Aβ protein, specifically bind to CD22, CD33, or CD74 that (a) promotes cis-trans conversion of CD22 or CD33 and/or (b) induces internalization of CD22, CD33 and CD74. The receptor binding portion of the bispecific antibodies and antigen-binding fragment provided herein can be used in Aβ removal, reduction of neuroinflammation, or both. The bispecific antibodies and antigen-binding fragment provided herein can also be used in the treatment of a disease or disorder that is associated with Aβ and/or neuroinflammation. In some embodiments, the bispecific antibodies used in methods provided herein specifically bind human CD22. In some embodiments, the bispecific antibodies used in methods provided herein specifically bind human CD33. In some embodiments, the bispecific antibodies used in methods provided herein specifically bind human CD74.

In some embodiments, the anti-CD22, anti-CD33 or anti-CD74 bispecific antibody that can be used in methods disclosed herein is an IgA, IgD, IgE, IgG, or IgM antibody. In some embodiments, the bispecific antibody is an IgA antibody. In some embodiments, the bispecific antibody is an IgD antibody. In some embodiments, the bispecific antibody is an IgE antibody. In some embodiments, the bispecific antibody is an IgG antibody. In some embodiments, the bispecific antibody is an IgM antibody. In some embodiments, the bispecific antibodies provided herein can be an IgG1 antibody, an IgG2 antibody, an IgG3 antibody, or an IgG4 antibody. In some embodiments, the bispecific antibody is an IgG1 antibody. In some embodiments, the bispecific antibody is an IgG2 antibody. In some embodiments, the bispecific antibody is an IgG3 antibody. In some embodiments, the bispecific antibody is an IgG4 antibody. In certain embodiments, the bispecific antibody comprises a heavy chain constant region, such as an IgG1, IgG2, IgG3, IgG4, IgA, IgE, IgM or IgD constant region or any of the above constant region with the glycosylation site and/or the glycoforms at the glycosylation site modified.

In some embodiments, used in methods disclosed herein are antigen-binding fragments of an anti-CD22 antibody, anti-CD33 antibody or anti-CD74 antibody. In some embodiments, antigen-binding fragments provided herein can be a single domain antibody (sdAb), a heavy chain antibody (HCAb), a Fab, a Fab′, a F(ab′) 2, a Fv, a single-chain variable fragment (scFv), or a (scFv) 2. In some embodiments, the antigen-binding fragment of an anti-CD22, anti-CD33 or anti-CD74 antibody is a single domain antibody (sdAb). In some embodiments, the antigen-binding fragment of an anti-CD22, anti-CD33 or anti-CD74 antibody is a heavy chain antibody (HCAb). In some embodiments, the antigen-binding fragment of an anti-CD22, anti-CD33 or anti-CD74 antibody is a Fab. In some embodiments, the antigen-binding fragment of an anti-CD22, anti-CD33 or anti-CD74 antibody is a Fab′. In some embodiments, the antigen-binding fragment of an anti-CD22, anti-CD33 or anti-CD74 antibody is a F(ab′)₂. In some embodiments, the antigen-binding fragment of an anti-CD22, anti-CD33 or anti-CD74 antibody is a Fv. In some embodiments, the antigen-binding fragment of an anti-CD22, anti-CD33 or anti-CD74 antibody is a scFv. In some embodiments, the antigen-binding fragment of an anti-CD22, anti-CD33 or anti-CD74 antibody is a disulfide-linked scFv [(scFv) 2]. In some embodiments, the antigen-binding fragment of an anti-CD22, anti-CD33 or anti-CD74 antibody is a diabody (dAb).

In some embodiments, used in methods disclosed herein are recombinant anti-CD22, anti-CD33 or anti-CD74 antibodies or antigen-binding fragments. In some embodiments, the anti-CD22, anti-CD33 or anti-CD74 antibodies or antigen-binding fragments provided herein comprise monoclonal antibodies or antigen-binding fragments. In some embodiments, the anti-CD22, anti-CD33 or anti-CD74 antibodies or antigen-binding fragments provided herein comprise polyclonal antibodies or antigen-binding fragments. In some embodiments, the anti-CD22, anti-CD33 or anti-CD74 antibodies or antigen-binding fragments provided herein comprise camelid (e.g., camels, dromedary and llamas) antibodies or antigen-binding fragments. In some embodiments, the anti-CD22, anti-CD33 or anti-CD74 antibodies or antigen-binding fragments provided herein comprise chimeric antibodies or antigen-binding fragments. In some embodiments, the anti-CD22, anti-CD33 or anti-CD74 antibodies or antigen-binding fragments provided herein comprise humanized antibodies or antigen-binding fragments. In some embodiments, the anti-CD22, anti-CD33 or anti-CD74 antibodies or antigen-binding fragments provided herein comprise human antibodies or antigen-binding fragments. In some embodiments, provided herein are anti-CD22, anti-CD33 or anti-CD74 humanized scFvs. In some embodiments, provided herein are anti-CD22, anti-CD33 or anti-CD74 human humanized Fabs.

In some embodiments, the anti-CD22, anti-CD33 or anti-CD74 antibodies or antigen-binding fragments used in the methods provided herein are isolated. In some embodiments, the anti-CD22, anti-CD33 or anti-CD74 antibodies or antigen-binding fragments used in the methods provided herein are substantially pure.

In some embodiments, the anti-CD22, anti-CD33 or anti-CD74 antibody or antigen-binding fragment used in methods provided herein comprises a monovalent antigen-binding site. In some embodiments, an anti-CD22, anti-CD33 or anti-CD74 antibody or antigen-binding fragment comprises a monospecific binding site. In some embodiments, an anti-CD22, anti-CD33 or anti-CD74 antibody or antigen-binding fragment comprises a bivalent binding site.

In some embodiments, a monoclonal antibody is modified by using recombinant DNA technology to generate alternative antibodies. In some embodiments, the constant domains of the light chain and heavy chain of a mouse monoclonal antibody are replaced with the constant regions of a human antibody to generate a chimeric antibody. In some embodiments, the constant regions are truncated or removed to generate a desired antibody fragment of a monoclonal antibody. In some embodiments, site-directed or high-density mutagenesis of the variable region(s) is used to optimize specificity and/or affinity of a monoclonal antibody.

In some embodiments, an anti-CD22, anti-CD33 or anti-CD74 antibody or antigen-binding fragment used in methods disclosed herein is a humanized antibody or antigen-binding fragment. Various methods for generating humanized antibodies are known in the art. Methods are known in the art for achieving high affinity binding with humanized antibodies. A non-limiting example of such a method is hypermutation of the variable region and selection of the cells expressing such high affinity antibodies (affinity maturation). In addition to the use of display libraries, the specified antigen (e.g., recombinant CD22, recombinant CD33, recombinant CD74 or an epitope of the respective recombinant proteins thereof) can be used to immunize a non-human animal, e.g., a rodent. In certain embodiments, rodent antigen-binding fragments (e.g., mouse antigen-binding fragments) can be generated and isolated using methods known in the art and/or disclosed herein. In some embodiments, a mouse can be immunized with an antigen (e.g., recombinant CD22, recombinant CD33, recombinant CD74 or an epitope of the respective recombinant proteins thereof).

In some embodiments, an anti-CD22, anti-CD33 or anti-CD74 antibody or antigen-binding fragment used in methods disclosed herein is a human antibody or antigen-binding fragment. Human antibodies can be prepared using various techniques known in the art. In some embodiments, human antibodies are generated from immortalized human B lymphocytes immunized in vitro. In some embodiments, human antibodies are generated from lymphocytes isolated from an immunized individual. In any case, cells that produce an antibody directed against a target antigen can be generated and isolated. In some embodiments, a human antibody is selected from a phage library, where that phage library expresses human antibodies. Alternatively, phage display technology can be used to produce human antibodies and antibody fragments in vitro, from immunoglobulin variable region gene repertoires from unimmunized donors. Techniques for the generation and use of antibody phage libraries are well-known in the art. Once antibodies are identified, affinity maturation strategies known in the art, including but not limited to, chain shuffling and site-directed mutagenesis, can be employed to generate higher affinity human antibodies. In some embodiments, human antibodies are produced in transgenic mice that contain human immunoglobulin loci. Upon immunization these mice are capable of producing the full repertoire of human antibodies in the absence of endogenous immunoglobulin production.

The specific CDR sequences defined herein are generally based on a combination of Kabat and Chothia definitions. However, it is understood that reference to a heavy chain CDR or CDRs and/or a light chain CDR or CDRs of a specific antibody encompass all CDR definitions as known to those of skill in the art. Anti-CD22 antibodies or antigen-binding fragments that can be used in methods provided herein include SM03 and SM06, and their variations and derivations.

The term “SM03” refers to a chimeric antibody against human CD22 (hCD22). Sequence features of SM03 are provided in the table below. Additional description of the structural and functional features of SM03 can be found in, e.g., Yang et al. (2006), Chinese J New Drug 15 (3): 186-92; and Chinese Pat. No. ZL03123054.7, incorporated herein in their entirety by reference.

The term “SM06” refers to a framework-patched or humanized version of chimeric antibody SM03 reengineered to reduce its potential immunogenicity and exhibiting affinity and specificity against hCD22. Sequence features of SM06 are provided in the table below. Additional description of the structural and functional features of SM06 can be found in, e.g., Liang et al. (2006) Chinese J New Drug 15 (21): 1832-36; Chinese Patent No. ZL 01144894.6, and U.S. Pat. No. 7,321,026 B2 &7,338,659 B2, incorporated herein in their entirety by reference.

TABLE 1
CDR sequences of SM03 and SM06

| Antibody | VL CDR1 | VL CDR2 | VL CDR3 |
|---|---|---|---|
| SM03/SM06 | RASQDISNYLN | YTSILHS | QQGNTLPWT |
| | (SEQ ID NO: 1) | (SEQ ID NO: 2) | (SEQ ID NO: 3) |
| | VH CDR1 | VH CDR2 | VH CDR3 |
| | IYDMS | YISSGGGTTYYPDTVKG | HSGYGSSYGVLFAY |
| | (SEQ ID NO: 4) | (SEQ ID NO: 5) | (SEQ ID NO: 6) |

TABLE 2
Variable regions of SM03 and SM06

| Antibody | VL | VH |
|---|---|---|
| SM03 | DIQLTQTTSSLSASLGDRVTISCRAS | QVQLQESGGGLVKPGGSLKLSCAAS |
| | QDISNYLNWYQQKPDGTVKLLIYYT | GFAFSIYDMSWVRQTPEKRLEWVA |
| | SILHSGVPSRFSGSGSGTDYSLTISNL | YISSGGGTTYYPDTVKGRFTISRDNA |
| | EQEDFATYFCQQGNTLPWTFGGGT | KNTLYLQMSSLKSEDTAMYYCARH |
| | KLEIKR | SGYGSSYGVLFAYWGQGTLVT |
| | (SEQ ID NO: 7) | VSS |
| | | (SEQ ID NO: 8) |
| SM06 | DIQLTQSPSSLSASVGDRVTISCRAS | EVQLVESGGGLVQPGGSLRLSCAAS |
| | QDISNYLNWYQQKPGKAPKLLIYYT | GFSFSIYDMSWVRQAPGKGLEWVA |
| | SILHSGVPSRFSGSGSGTEFTLTISSL | YISSGGGTTYYPDTVKGRFTISRDNA |
| | QPEDFATYFCQQGNTLPWTFGGGT | KNSLYLQMNSLRVEDTALYYCARH |
| | KVEIKR | SGYGSSYGVLFAYWGQGTLVT |
| | (SEQ ID NO: 9) | VSS |
| | | (SEQ ID NO: 10) |

In some embodiments, anti-CD22 antibodies or antigen-binding fragments that can be used in methods provided herein comprise one, two, three, four, five, and/or six CDRs of SM03/SM06. In some embodiments, the anti-CD22 antibodies or antigen-binding fragments comprise a VL comprising one, two, and/or three, VL CDRs from Table 1. In some embodiments, the anti-CD22 antibodies or antigen-binding fragments provided herein comprise a VH comprising one, two, and/or three VH CDRs from Table 1. In some embodiments, the anti-CD22 antibodies or antigen-binding fragments provided herein comprise one, two, and/or three VL CDRs and one, two, and/or three VH CDRs from Table 1.

The term “LL2” refers to a chimeric antibody against human CD22 (hCD22). Sequence features of LL2 are provided in the table below. Additional description of the structural and functional features of LL2 can be found in, e.g., Leung et al. (1994) Chimerization of LL2, a rapidly internalizing antibody specific for B-cell lymphoma. Hybridoma 13:469-476; Stein R, et. al. (1993) Epitope specificity of the anti-(B cell lymphoma) monoclonal antibody, LL2. Cancer Immunol Immunother; 37 (5): 293-8; U.S. Pat. No. 5,789,554A incorporated herein in their entirety by reference. The term “hLL2” refers to a framework-patched or humanized version of chimeric antibody LL2 reengineered to reduce its potential immunogenicity and exhibiting affinity and specificity against hCD22. Sequence features of hLL2 are provided in the table below. Additional description of the structural and functional features of hLL2 can be found in, e.g., Leung, et al. (1995) Construction and characterization of a humanized, internalizing B-cell (CD22)-specific, leukemia/lymphoma antibody, LL2. Mol. Immunol. 32:1413-1427; Juweid M et. al. (1995) Treatment of non-Hodgkin's lymphoma with radiolabeled murine, chimeric, or humanized LL2, an anti-CD22 monoclonal antibody. Cancer Res. 55 (23 Suppl): 5899s-5907s; U.S. Pat. No. 5,789,554A, incorporated herein in their entirety by reference.

TABLE 3
CDR sequences of LL2 and hLL2

| Antibody | VL CDR1 | VL CDR2 | VL CDR3 |
|---|---|---|---|
| LL2/hLL2 | KSSQSVLYSANHK | WASTRES | HQYLSSWT |
| | NYLA | (SEQ ID NO: 12) | (SEQ ID NO: 13) |
| | (SEQ ID NO: 11) | | |
| | VH CDR1 | VH CDR2 | VH CDR3 |
| | SYWLH | YINPRNDYTEYNQNFKD | RDITTFY |
| | (SEQ ID NO: 14) | (SEQ ID NO: 15) | (SEQ ID NO: 16) |

TABLE 4
Variable regions of LL2 and hLL2

| Antibody | VL | VH |
|---|---|---|
| LL2 | DIQLTQSPSSLAVSAGENVTMSCKSS | QVQLQESGAELSKPGASVKMSCKA |
| | QSVLYSANHKNYLAWYQQKPGQSP | SGYTETSYWLHWIKQRPGQGLEWI |
| | KLLIYWASTRESGVPDRFTGSGSGT | GYINPRNDYTEYNQNFKDKATLTA |
| | DFTLTISRVQVEDLAIYYCHQYLSS | DKSSSTAYMQLSSLTSEDSAVYYCA |
| | WTGGGTKLEIK | RRDITTFYWGQGTTLTVSS |
| | (SEQ ID NO: 17) | (SEQ ID NO: 18) |
| hLL2 | DIQLTQSPSSLSASVGDRVTMSCKSS | QVQLVQSGAEVKKPGSSVKVSCKA |
| | QSVLYSANHKNYLAWYQQKPGKA | SGYTFTSYWLHWVRQAPGQGLEWI |
| | PKLLIYWASTRESGVPSRFSGSGSGT | GYINPRNDYTEYNQNFKDKATITAD |
| | DFTFTISSLQPEDIATYYCHQYLSSW | ESTNTAYMELSSLRSEDTAFYFCAR |
| | TFGGGTKLEIK | RDITTFYWGQGTTVTVSS |
| | (SEQ ID NO: 19) | (SEQ ID NO: 20) |

In some embodiments, anti-CD22 antibodies or antigen-binding fragments that can be used in methods provided herein comprise one, two, three, four, five, and/or six CDRs of LL2/hLL2. In some embodiments, the anti-CD22 antibodies or antigen-binding fragments comprise a VL comprising one, two, and/or three, VL CDRs from Table 3. In some embodiments, the anti-CD22 antibodies or antigen-binding fragments provided herein comprise a VH comprising one, two, and/or three VH CDRs from Table 3. In some embodiments, the anti-CD22 antibodies or antigen-binding fragments provided herein comprise one, two, and/or three VL CDRs and one, two, and/or three VH CDRs from Table 3

The term “Gemtuzumab” refers to a framework-patched or humanized version of anti-CD33 antibody reengineered to reduce its potential immunogenicity and exhibiting affinity and specificity against hCD33. Sequence features of Gemtuzumab are provided in the table below. Additional description of the structural and functional features of Gemtuzumab can be found in, e.g., Bernstein ID (2000). Monoclonal antibodies to the myeloid stem cells: therapeutic implications of CMA-676, a humanized anti-CD33 antibody calicheamicin conjugate. Leukemia. 14 (3): 474-5; U.S. Pat. No. 5,877,296A, incorporated herein in their entirety by reference.

TABLE 5
CDR sequences of Gemtuzumab

| Antibody | VL CDR1 | VL CDR2 | VL CDR3 |
|---|---|---|---|
| Gemtuzumab | RASESLDNYGIR | AASNQGS | QQTKEVPWS |
| | FLT | (SEQ ID NO: 22) | (SEQ ID NO: 23) |
| | (SEQ ID NO: 21) | | |
| | VH CDR1 | VH CDR2 | VH CDR3 |
| | DSNIH | YIYPYNGGTDYNQKFKN | GNPWLAY |
| | (SEQ ID NO: 24) | (SEQ ID NO: 25) | (SEQ ID NO: 26) |

TABLE 6
Variable regions of Gemtuzumab

| Antibody | VL | VH |
|---|---|---|
| Gemtuzumab | DIQLTQSPSTLSASVGDRVTITCRAS | EVQLVQSGAEVKKPGSSVKVSCKA |
| | ESLDNYGIRFLTWFQQKPGKAPKLL | SGYTITDSNIHWVRQAPGQSLEWIG |
| | MYAASNQGSGVPSRESGSGSGTEFT | YIYPYNGGTDYNQKEKNRATLTVD |
| | LTISSLQPDDFATYYCQQTKEVPWS | NPTNTAYMELSSLRSEDTAFYYCVN |
| | FGQGTKVEVKR | GNPWLAYWGQGTLVTVSS |
| | (SEQ ID NO: 27) | (SEQ ID NO: 28) |

In some embodiments, anti-CD33 antibodies or antigen-binding fragments that can be used in methods provided herein comprise one, two, three, four, five, and/or six CDRs of Gemtuzumab. In some embodiments, the anti-CD33 antibodies or antigen-binding fragments comprise a VL comprising one, two, and/or three, VL CDRs from Table 5. In some embodiments, the anti-CD33 antibodies or antigen-binding fragments provided herein comprise a VH comprising one, two, and/or three VH CDRs from Table 5. In some embodiments, the anti-CD33 antibodies or antigen-binding fragments provided herein comprise one, two, and/or three VL CDRs and one, two, and/or three VH CDRs from Table 5.

The term “HuMy9-6” refers to a framework-patched or humanized version of anti-CD33 antibody reengineered to reduce its potential immunogenicity and exhibiting affinity and specificity against hCD33. Sequence features of HuMy9-6 are provided in the table below. Additional description of the structural and functional features of HuMy9-6 can be found in, e.g., Lapusan Set. al. (2012) Phase I studies of AVE9633, an anti-CD33 antibody-maytansinoid conjugate, in adult patients with relapsed/refractory acute myeloid leukemia. Invest New Drugs. 30 (3): 1121-31; U.S. Pat. No. U.S. Pat. No. 10,000,566B2, incorporated herein in their entirety by reference.

TABLE 7
CDR sequences of HuMy9-6

| Antibody | VL CDR1 | VL CDR2 | VL CDR3 |
|---|---|---|---|
| HuMy9-6 | KSSQSVFFSSSQK | WASTRES | HQYLSSRT |
| | NYLA | (SEQ ID NO: 30) | (SEQ ID NO: 31) |
| | (SEQ ID NO: 29) | | |
| | VH CDR1 | VH CDR2 | VH CDR3 |
| | SYYIH | VIYPGNDDISYNQKFQG | EVRLRYFDV |
| | (SEQ ID NO: 32) | (SEQ ID NO: 33) | (SEQ ID NO: 34) |

TABLE 8
Variable regions of HuMy9-6

| Antibody | VL | VH |
|---|---|---|
| HuMy9-6 | EIVLTQSPGSLAVSPGERVTMSCKS | QVQLQQPGAEVVKPGASVKMSCKA |
| | SQSVFFSSSQKNYLAWYQQIPGQSP | SGYTFTSYYIHWIKQTPGQGLEWVG |
| | RLLIYWASTRESGVPDRFTGSGSGT | VIYPGNDDISYNQKFOGKATLTADK |
| | DFTLTISSVQPEDLAIYYCHQYLSS | SSTTAYMQLSSLTSEDSAVYYCARE |
| | RTFGQGTKLEIKR | VRLRYFDVWGQGTTVTTVSS |
| | (SEQ ID NO: 35) | (SEQ ID NO: 36) |

In some embodiments, anti-CD33 antibodies or antigen-binding fragments that can be used in methods provided herein comprise one, two, three, four, five, and/or six CDRs of HuMy9-6. In some embodiments, the anti-CD33 antibodies or antigen-binding fragments comprise a VL comprising one, two, and/or three, VL CDRs from Table 7. In some embodiments, the anti-CD33 antibodies or antigen-binding fragments provided herein comprise a VH comprising one, two, and/or three VH CDRs from Table 7. In some embodiments, the anti-CD33 antibodies or antigen-binding fragments provided herein comprise one, two, and/or three VL CDRs and one, two, and/or three VH CDRs from Table 7.

The term “Lintuzumab” refers to a framework-patched or humanized version of anti-CD33 antibody reengineered to reduce its potential immunogenicity and exhibiting affinity and specificity against hCD33. Sequence features of Lintuzumab are provided in the table below. Additional description of the structural and functional features of Lintuzumab can be found in, e.g., Gibson AD. (2002) Phase III trial of a humanized anti-CD33 antibody (HuM195) in patients with relapsed or refractory acute myeloid leukemia. Clin Lymphoma. 3 (1): 18-9; U.S. Pat. No. U.S. Pat. No. 9,061,074B2, incorporated herein in their entirety by reference.

TABLE 9
CDR sequences of Lintuzumab

| Antibody | VL CDR1 | VL CDR2 | VL CDR3 |
|---|---|---|---|
| Lintuzumab | RASESVDNYGI | AASNQGS | QQSKEVPWT |
| | SFMN | (SEQ ID NO: 38) | (SEQ ID NO: |
| | (SEQ ID NO: | | 39) |
| | 37) | | |
| | VH CDR1 | VH CDR2 | VH CDR3 |
| | DYNMH | YIYPYNGGTGYNQK | GRPAMDY |
| | (SEQ ID NO: | FKS | (SEQ ID NO: |
| | 40) | (SEQ ID NO: 41) | 42) |

TABLE 10
Variable regions of Lintuzumab

| Antibody | VL | VH |
|---|---|---|
| Lintuzumab | DIQMTQSPSSLSASVGDRVTITCRAS | QVQLVQSGAEVKKPGSSVKVSCKA |
| | ESVDNYGISFMNWEQQKPGKAPKL | SGYTFTDYNMHWVRQAPGQGLEWI |
| | LIYAASNQGSGVPSRFSGSGSGTDF | GYIYPYNGGTGYNQKFKSKATITAD |
| | TLTISSLQPDDFATYYCQQSKEVPW | ESTNTAYMELSSLRSEDTAVYYCAR |
| | TFGQGTKVEIK | GRPAMDYWGQGTLVTVSS |
| | (SEQ ID NO: 43) | (SEQ ID NO: 44) |

In some embodiments, anti-CD33 antibodies or antigen-binding fragments that can be used in methods provided herein comprise one, two, three, four, five, and/or six CDRs of Lintuzumab. In some embodiments, the anti-CD33 antibodies or antigen-binding fragments comprise a VL comprising one, two, and/or three, VL CDRs from Table 9. In some embodiments, the anti-CD33 antibodies or antigen-binding fragments provided herein comprise a VH comprising one, two, and/or three VH CDRs from Table 9. In some embodiments, the anti-CD33 antibodies or antigen-binding fragments provided herein comprise one, two, and/or three VL CDRs and one, two, and/or three VH CDRs from Table 9.

The term “cLL1” refers to a chimeric antibody against human CD74 (hCD74). Sequence features of LL1 are provided in the table below. Additional description of the structural and functional features of LL1 can be found in, e.g., Stein R et. al. (2007) CD74: a new candidate target for the immunotherapy of B-cell neoplasms. Clin Cancer Res. 13 (18 Pt 2): 5556s-5563s; U.S. Pat. No. U.S. Pat. No. 8,119,101B2 incorporated herein in their entirety by reference.

The term “hLL1” refers to a framework-patched or humanized version of anti-CD74 antibody reengineered to reduce its potential immunogenicity and exhibiting affinity and specificity against hCD74. Sequence features of hLL1 are provided in the table below. Additional description of the structural and functional features of hLL1 can be found in, e.g., Stein R et. al. (2007) CD74: a new candidate target for the immunotherapy of B-cell neoplasms. Clin Cancer Res. 13 (18 Pt 2): 5556s-5563s; U.S. Pat. No. 8,119,101B2 incorporated herein in their entirety by reference.

TABLE 11
CDR sequences of cLL1/hLL1

| Antibody | VL CDR1 | VL CDR2 | VL CDR3 |
|---|---|---|---|
| cLL1/hLL1 | RSSQSLVHRNGN | TVSNRFS | SQSSHVPPT |
| | TYLH | (SEQ ID NO: 46) | (SEQ ID NO: |
| | (SEQ ID NO: | | 47) |
| | 45) | | |
| | VH CDR1 | VH CDR2 | VH CDR3 |
| | NYGVN | WINPNTGEPTFDDD | SRGKNEAWFAY |
| | (SEQ ID NO: | FKG | (SEQ ID NO: |
| | 48) | (SEQ ID NO: 49) | 50) |

TABLE 12
Variable regions of cLL1/hLL1

| Antibody | VL | VH |
|---|---|---|
| cLL1 | DIQLTQTPLSLPVSLGDQASISCRSS | QVQLQQSGPELKKPGETVKVTCKTS |
| | QSLVHRNGNTYLHWYLQKPGQSPK | GYTFTNYGVNWIKQTPGEGLQWM |
| | LLIYTVSNRFSGVPDRESGSGSGTDF | GWINPNTGEPTFDDDFKGRFAFSLE |
| | TLKISRVEAEDLGLYFCSQSSHVPPT | SSASTAFLQISNLKNEDMGTYFCSRS |
| | FGAGTKLEIKR | RGKNEAWFAYWGOGTLVTVSS |
| | (SEQ ID NO: 51) | (SEQ ID NO: 52) |
| hLL1 | DIQLTQSPLSLPVTLGQPASISCRSS | QVQLQQSGSELKKPGASVKVSCKA |
| | QSLVHRNGNTYLHWFQQRPGQSPR | SGYTFTNYGVNWIKQAPGQGLQW |
| | LLIYTVSNRFSGVPDRFSGSGSGTDF | MGWINPNTGEPTFDDDFKGRFAFSL |
| | TLKISRVEAEDVGVYFCSQSSHVPP | DTSVSTAYLQISSLKADDTAVYFCS |
| | TFGAGTRLEIKR | RSRGKNEAWFAYWGQGTLVTVSS |
| | (SEQ ID NO: 53) | (SEQ ID NO: 54) |

In some embodiments, anti-CD74 antibodies or antigen-binding fragments that can be used in methods provided herein comprise one, two, three, four, five, and/or six CDRs of cLL1/hLL1. In some embodiments, the anti-CD74 antibodies or antigen-binding fragments comprise a VL comprising one, two, and/or three, VL CDRs from Table 11. In some embodiments, the anti-CD74 antibodies or antigen-binding fragments provided herein comprise a VH comprising one, two, and/or three VH CDRs from Table 11. In some embodiments, the anti-CD74 antibodies or antigen-binding fragments provided herein comprise one, two, and/or three VL CDRs and one, two, and/or three VH CDRs from Table 11.

The term “Aducanumab” refers to a framework-patched or humanized version of anti-Aβantibody reengineered to reduce its potential immunogenicity and exhibiting affinity and specificity against Aβ. Sequence features of Aducanumab are provided in the table below. Additional description of the structural and functional features of Aducanumab can be found in, e.g., Sevigny J et. al. (2016) The antibody aducanumab reduces Aβ plaques in Alzheimer's disease. Nature. 537 (7618): 50-6; U.S. Pat. No. U.S. Pat. No. 9,670,272B2 incorporated herein in their entirety by reference.

TABLE 13
CDR sequences of Aducanumab

| Antibody | VL CDR1 | VL CDR2 | VL CDR3 |
|---|---|---|---|
| Aducanumab | RASQSISSYLN | AASSLQS | QQSYSTPLT |
| | (SEQ ID NO: | (SEQ ID NO: 56) | (SEQ ID NO: |
| | 55) | | 57) |
| | VH CDR1 | VH CDR2 | VH CDR3 |
| | SYGMH | VIWFDGTKKYYTDS | DRGIGARRGPYY |
| | (SEQ ID NO: | VKG | MDV (SEQ ID |
| | 58) | (SEQ ID NO: 59) | NO: 60) |

TABLE 14
Variable regions of Aducanumab

| Antibody | VL | VH |
|---|---|---|
| Aducanumab | DIQMTQSPSSLSASVGDRVTITCRAS | QVQLVESGGGVVQPGRSLRLSCAAS |
| | QSISSYLNWYQQKPGKAPKLLIYAA | GFAFSSYGMHWVRQAPGKGLEWV |
| | SSLQSGVPSRFSGSGSGTDFTLTISSL | AVIWFDGTKKYYTDSVKGRFTISRD |
| | QPEDFATYYCQQSYSTPLTFGGGTK | NSKNTLYLQMNTLRAEDTAVYYCA |
| | VEIKR | RDRGIGARRGPYYMDVWGKGTTVT |
| | (SEQ ID NO: 61) | VSS |
| | | (SEQ ID NO: 62) |
| | | EVQLVESGGGVVQPGRSLRLSCAAS |
| | | GFAFSSYGMHWVRQAPGKGLEWV |
| | | AVIWFDGTKKYYTDSVKGRFTISRD |
| | | NSKNTLYLQMNTLRAEDTAVYYCA |
| | | RDRGIGARRGPYYMDVWGKGTTVT |
| | | VSS |
| | | (SEQ ID NO: 63) |

In some embodiments, anti-Aβ antibodies or antigen-binding fragments that can be used in methods provided herein comprise one, two, three, four, five, and/or six CDRs of Aducanumab. In some embodiments, the anti-Aβ antibodies or antigen-binding fragments comprise a VL comprising one, two, and/or three, VL CDRs from Table 13 In some embodiments, the anti-Aβ antibodies or antigen-binding fragments provided herein comprise a VH comprising one, two, and/or three VH CDRs from Table 13. In some embodiments, the anti-Aβ antibodies or antigen-binding fragments provided herein comprise one, two, and/or three VL CDRs and one, two, and/or three VH CDRs from Table 13.

The term “BAN2401” refers to a framework-patched or humanized version of anti-Aβantibody reengineered to reduce its potential immunogenicity and exhibiting affinity and specificity against Aβ. Sequence features of BAN2401 are provided in the table below. Additional description of the structural and functional features of BAN2401 can be found in, e.g., Tucker S et. al. (2015) The murine version of BAN2401 (mAb158) selectively reduces amyloid-β protofibrils in brain and cerebrospinal fluid of tg-ArcSwe mice. J Alzheimers Dis. 43 (2): 575-88; U.S. Pat. No. 8,025,878B2 incorporated herein in their entirety by reference.

TABLE 15
CDR sequences of BAN2401

| Antibody | VL CDR1 | VL CDR2 | VL CDR3 |
|---|---|---|---|
| BAN2401 | RSSQSIVHSNGN | KVSNRFS | FQGSHVPPT |
| | TYLE | (SEQ ID NO: 65) | (SEQ ID NO: |
| | (SEQ ID NO: | | 66) |
| | 64) | | |
| | VH CDR1 | VH CDR2 | VH CDR3 |
| | SFGMH | YISSGSSTIYYGDT | EGGYYYGRSYYT |
| | (SEQ ID NO: | VKG | MDY (SEQ ID |
| | 67) | (SEQ ID NO: 68) | NO: 69) |

TABLE 16
Variable regions of BAN2401

| Antibody | VL | VH |
|---|---|---|
| BAN2401 | DVVMTQSPLSLPVTPGAPASISCRSS | EVQLVESGGGLVQPGGSLRLSCSAS |
| | QSIVHSNGNTYLEWYLQKPGQSPK | GFTFSSFGMHWVRQAPGKGLEWVA |
| | LLIYKVSNRFSGVPDRESGSGSGTD | YISSGSSTIYYGDTVKGRFTISRDNA |
| | FTLRISRVEAEDVGIYYCFQGSHVPP | KNSLFLQMSSLRAEDTAVYYCARE |
| | TFGPGTKLEIK | GGYYYGRSYYTMDYWGQGTTVTV |
| | (SEQ ID NO: 70) | S |
| | | (SEQ ID NO: 71) |

In some embodiments, anti-Aβ antibodies or antigen-binding fragments that can be used in methods provided herein comprise one, two, three, four, five, and/or six CDRs of BAN2401. In some embodiments, the anti-Aβ antibodies or antigen-binding fragments comprise a VL comprising one, two, and/or three, VL CDRs from Table 15 In some embodiments, the anti-Aβ antibodies or antigen-binding fragments provided herein comprise a VH comprising one, two, and/or three VH CDRs from Table 15. In some embodiments, the anti-Aβ antibodies or antigen-binding fragments provided herein comprise one, two, and/or three VL CDRs and one, two, and/or three VH CDRs from Table 15.

The term “Gantenerumab” refers to a framework-patched or humanized version of anti-Aβantibody reengineered to reduce its potential immunogenicity and exhibiting affinity and specificity against Aβ. Sequence features of Gantenerumab are provided in the table below. Additional description of the structural and functional features of Gantenerumab can be found in, e.g., Bohrmann B et. al. (2012) Gantenerumab: a novel human anti-Aβ antibody demonstrates sustained cerebral amyloid-βbinding and elicits cell-mediated removal of human amyloid-β. J Alzheimers Dis. 28 (1): 49-69; U.S. Pat. US7,794,719B2 incorporated herein in their entirety by reference.

TABLE 17
CDR sequences of Gantenerumab

| Antibody | VL CDR1 | VL CDR2 | VL CDR3 |
|---|---|---|---|
| Gantenerumab | RASQSVSSSYLA | GASSRAT | LQIYNMPIT |
| | (SEQ ID NO: | (SEQ ID NO: | (SEQ ID NO: |
| | 72) | 73) | 74) |
| | VH CDR1 | VH CDR2 | VH CDR3 |
| | SYAMS | INASGTRTYYAD | GKGNTHKPYGYV |
| | (SEQ ID NO: | SVKG (SEQ ID | RYFDV (SEQ |
| | 75) | NO: 76) | ID NO: 77) |

TABLE 18
Variable regions of Gantenerumab

| Antibody | VL | VH |
|---|---|---|
| Gantenerumab | DIVLTQSPATLSLSPGERATLSCR | QVQLVESGGGLVQPGGSLRLSCAAS |
| | ASQSVSSSYLAWYQQKPGQAPRL | GFTFSSYAMSWVRQAPGKGLEWVA |
| | LIYGASSRATGVPARFSGSGSGTD | INASGTRTYYADSVKGRFTISRDNS |
| | FTLTISSLEPEDFATYYCLQIYNMP | KNTLYLQMNLRAEDTAVYYCARG |
| | ITFGQGTKVEIKRT | KGNTHKPYGYVRYFDVWGQGTLV |
| | (SEQ ID NO: 78) | TVSS |
| | | (SEQ ID NO: 79) |

In some embodiments, anti-Aβ antibodies or antigen-binding fragments that can be used in methods provided herein comprise one, two, three, four, five, and/or six CDRs of Gantenerumab. In some embodiments, the anti-Aβ antibodies or antigen-binding fragments comprise a VL comprising one, two, and/or three, VL CDRs from Table 17 In some embodiments, the anti-Aβ antibodies or antigen-binding fragments provided herein comprise a VH comprising one, two, and/or three VH CDRs from Table 17. In some embodiments, the anti-Aβ antibodies or antigen-binding fragments provided herein comprise one, two, and/or three VL CDRs and one, two, and/or three VH CDRs from Table 17.

The term “Crenezumab” refers to a framework-patched or humanized version of anti-Aβantibody reengineered to reduce its potential immunogenicity and exhibiting affinity and specificity against Aβ. Sequence features of Crenezumab are provided in the table below. Additional description of the structural and functional features of Crenezumab can be found in, e.g., Bohrmann B et. al. (2012) Gantenerumab: a novel human anti-Aβ antibody demonstrates sustained cerebral amyloid-β binding and elicits cell-mediated removal of human amyloid-β. J Alzheimers Dis. 28 (1): 49-69; U.S. Pat. No. US7, 794, 719B2 incorporated herein in their entirety by reference.

TABLE 19
CDR sequences of Crenezumab

| Antibody | VL CDR1 | VL CDR2 | VL CDR3 |
|---|---|---|---|
| Crenezumab | RASQSVSSSYLA | GASSRAT | LOIYNMPIT |
| | (SEQ ID NO: | (SEQ ID NO: | (SEQ ID NO: |
| | 80) | 81) | 82) |
| | VH CDR1 | VH CDR2 | VH CDR3 |
| | SYAMS | INASGTRTYYADS | GKGNTHKPYGYV |
| | (SEQ ID NO: | VKG (SEQ ID | RYFDV (SEQ |
| | 83) | NO: 84) | ID NO: 85) |

TABLE 20
Variable regions of Crenezumab

| Antibody | VL | VH |
|---|---|---|
| Crenezumab | DIVLTQSPATLSLSPGERATLSCR | QVQLVESGGGLVQPGGSLRLSCAAS |
| | ASQSVSSSYLAWYQQKPGQAPRL | GFTFSSYAMSWVRQAPGKGLEWVA |
| | LIYGASSRATGVPARFSGSGSGTD | INASGTRTYYADSVKGRFTISRDNS |
| | FTLTISSLEPEDFATYYCLQIYNMP | KNTLYLQMNLRAEDTAVYYCARG |
| | ITFGQGTKVEIKRT | KGNTHKPYGYVRYFDVWGQGTLV |
| | (SEQ ID NO: 86) | TVSS |
| | | (SEQ ID NO: 87) |

In some embodiments, anti-Aβ antibodies or antigen-binding fragments that can be used in methods provided herein comprise one, two, three, four, five, and/or six CDRs of Crenezumab. In some embodiments, the anti-Aβ antibodies or antigen-binding fragments comprise a VL comprising one, two, and/or three, VL CDRs from Table 19 In some embodiments, the anti-Aβ antibodies or antigen-binding fragments provided herein comprise a VH comprising one, two, and/or three VH CDRs from Table 19. In some embodiments, the anti-Aβ antibodies or antigen-binding fragments provided herein comprise one, two, and/or three VL CDRs and one, two, and/or three VH CDRs from Table 19.

The term “AD38” refers to a framework-patched or humanized version of anti-Aβ antibody reengineered to reduce its potential immunogenicity and exhibiting affinity and specificity against Aβ. Sequence features of AD38 are provided in the table below. Additional description of the structural and functional features of AD38 can be found in, e.g., WO Pat. No. WO2006, 103, 116A1 incorporated herein in their entirety by reference.

TABLE 21
CDR sequences of AD38

| Antibody | VL CDR1 | VL CDR2 | VL CDR3 |
|---|---|---|---|
| AD38 | RASQDISTSLA | GTSYRAT | QHRRDWPPGAT |
| | (SEQ ID NO: | (SEQ ID NO: | (SEQ ID NO: |
| | 88) | 89) | 90) |
| | VH CDR1 | VH CDR2 | VH CDR3 |
| | DYWMH | RLKSDGSSRSYADF | ELRGWSIEY |
| | (SEQ ID NO: | VKG (SEQ ID | (SEQ ID NO: |
| | 91) | NO: 92) | 93) |

TABLE 22
Variable regions of AD38

| Antibody | VL | VH |
|---|---|---|
| AD38 | DIVLTQSPATLSLSPGERATLSCR | QVQLVESGGGLVQPGGSLRLSCAAS |
| | ASQSVSSSYLAWYQQKPGQAPRL | GFTFSSYAMSWVRQAPGKGLEWVA |
| | LIYGASSRATGVPARFSGSGSGTD | INASGTRTYYADSVKGRFTISRDNS |
| | FTLTISSLEPEDFATYYCLQIYNMP | KNTLYLQMNLRAEDTAVYYCARG |
| | ITFGQGTKVEIKRT | KGNTHKPYGYVRYFDVWGQGTLV |
| | (SEQ ID NO: 94) | TVSS |
| | | (SEQ ID NO: 95) |

In some embodiments, anti-Aβ antibodies or antigen-binding fragments that can be used in methods provided herein comprise one, two, three, four, five, and/or six CDRs of AD38. In some embodiments, the anti-Aβ antibodies or antigen-binding fragments comprise a VL comprising one, two, and/or three, VL CDRs from Table 21 In some embodiments, the anti-Aβ antibodies or antigen-binding fragments provided herein comprise a VH comprising one, two, and/or three VH CDRs from Table 21. In some embodiments, the anti-Aβ antibodies or antigen-binding fragments provided herein comprise one, two, and/or three VL CDRs and one, two, and/or three VH CDRs from Table 21.

In some embodiments, the antibodies or antigen-binding fragments thereof that that can be used in methods provided herein comprise a light chain variable region (VL) comprising (1) a light chain CDR1 (VL CDR1) having the amino acid sequence of SEQ ID NO: 1; (2) a light chain CDR2 (VL CDR2) having the amino acid sequence of SEQ ID NO: 2; or (3) a light chain CDR3 (VL CDR3) having the amino acid sequence of SEQ ID NO: 3; or a variant thereof having up to about 3, about 5, about 8, about 10, about 12, or about 15 amino acid substitutions, additions, and/or deletions in the VL CDRs. In some embodiments, the variant has about 5 amino acid substitutions, additions, and/or deletions in the VL CDRs. In some embodiments, the antibodies or antigen-binding fragments thereof that specifically bind to CD22 comprise a VL comprising (1) a VL CDR1 having the amino acid sequence of SEQ ID NO: 1; (2) a VL CDR2 having the amino acid sequence of SEQ ID NO: 2; and (3) a VL CDR3 having the amino acid sequence of SEQ ID NO: 3; or a variant thereof having up to about 3, about 5, about 8, about 10, about 12, or about 15 amino acid substitutions, additions, and/or deletions in the VL CDRs. In some embodiments, the variant has up to about 5 amino acid substitutions, additions, and/or deletions in the VL CDRs.

In some embodiments, the antibodies or antigen-binding fragments thereof that that can be used in methods provided herein comprise a light chain variable region (VL) comprising (1) a light chain CDR1 (VL CDR1) having the amino acid sequence of SEQ ID NO: 11; (2) a light chain CDR2 (VL CDR2) having the amino acid sequence of SEQ ID NO: 12; or (3) a light chain CDR3 (VL CDR3) having the amino acid sequence of SEQ ID NO: 13; or a variant thereof having up to about 3, about 5, about 8, about 10, about 12, or about 15 amino acid substitutions, additions, and/or deletions in the VL CDRs. In some embodiments, the variant has about 5 amino acid substitutions, additions, and/or deletions in the VL CDRs. In some embodiments, the antibodies or antigen-binding fragments thereof that specifically bind to CD22 comprise a VL comprising (1) a VL CDR1 having the amino acid sequence of SEQ ID NO: 11; (2) a VL CDR2 having the amino acid sequence of SEQ ID NO: 12; and (3) a VL CDR3 having the amino acid sequence of SEQ ID NO: 13; or a variant thereof having up to about 3, about 5, about 8, about 10, about 12, or about 15 amino acid substitutions, additions, and/or deletions in the VL CDRs. In some embodiments, the variant has up to about 5 amino acid substitutions, additions, and/or deletions in the VL CDRs.

In some embodiments, the antibodies or antigen-binding fragments thereof that that can be used in methods provided herein comprise a light chain variable region (VL) comprising (1) a light chain CDR1 (VL CDR1) having the amino acid sequence of SEQ ID NO: 21; (2) a light chain CDR (VL CDR2) having the amino acid sequence of SEQ ID NO: 22; or (3) a light chain CDR3 (VL CDR3) having the amino acid sequence of SEQ ID NO: 23; or a variant thereof having up to about 3, about 5, about 8, about 10, about 12, or about 15 amino acid substitutions, additions, and/or deletions in the VL CDRs. In some embodiments, the variant has about 5 amino acid substitutions, additions, and/or deletions in the VL CDRs. In some embodiments, the antibodies or antigen-binding fragments thereof that specifically bind to CD33 comprise a VL comprising (1) a VL CDR1 having the amino acid sequence of SEQ ID NO: 21; (2) a VL CDR2 having the amino acid sequence of SEQ ID NO: 22; and (3) a VL CDR3 having the amino acid sequence of SEQ ID NO: 23; or a variant thereof having up to about 3, about 5, about 8, about 10, about 12, or about 15 amino acid substitutions, additions, and/or deletions in the VL CDRs. In some embodiments, the variant has up to about 5 amino acid substitutions, additions, and/or deletions in the VL CDRs.

In some embodiments, the antibodies or antigen-binding fragments thereof that that can be used in methods provided herein comprise a light chain variable region (VL) comprising (1) a light chain CDR1 (VL CDR1) having the amino acid sequence of SEQ ID NO: 29; (2) a light chain CDR (VL CDR2) having the amino acid sequence of SEQ ID NO: 30; or (3) a light chain CDR3 (VL CDR3) having the amino acid sequence of SEQ ID NO: 31; or a variant thereof having up to about 3, about 5, about 8, about 10, about 12, or about 15 amino acid substitutions, additions, and/or deletions in the VL CDRs. In some embodiments, the variant has about 5 amino acid substitutions, additions, and/or deletions in the VL CDRs. In some embodiments, the antibodies or antigen-binding fragments thereof that specifically bind to CD33 comprise a VL comprising (1) a VL CDR 1 having the amino acid sequence of SEQ ID NO: 29; (2) a VL CDR2 having the amino acid sequence of SEQ ID NO: 30; and (3) a VL CDR3 having the amino acid sequence of SEQ ID NO: 31; or a variant thereof having up to about 3, about 5, about 8, about 10, about 12, or about 15 amino acid substitutions, additions, and/or deletions in the VL CDRs. In some embodiments, the variant has up to about 5 amino acid substitutions, additions, and/or deletions in the VL CDRs.

In some embodiments, the antibodies or antigen-binding fragments thereof that that can be used in methods provided herein comprise a light chain variable region (VL) comprising (1) a light chain CDR1 (VL CDR1) having the amino acid sequence of SEQ ID NO: 37; (2) a light chain CDR (VL CDR2) having the amino acid sequence of SEQ ID NO: 38; or (3) a light chain CDR3 (VL CDR3) having the amino acid sequence of SEQ ID NO: 39; or a variant thereof having up to about 3, about 5, about 8, about 10, about 12, or about 15 amino acid substitutions, additions, and/or deletions in the VL CDRs. In some embodiments, the variant has about 5 amino acid substitutions, additions, and/or deletions in the VL CDRs. In some embodiments, the antibodies or antigen-binding fragments thereof that specifically bind to CD33 comprise a VL comprising (1) a VL CDR1 having the amino acid sequence of SEQ ID NO: 37; (2) a VL CDR2 having the amino acid sequence of SEQ ID NO: 38; and (3) a VL CDR3 having the amino acid sequence of SEQ ID NO: 39; or a variant thereof having up to about 3, about 5, about 8, about 10, about 12, or about 15 amino acid substitutions, additions, and/or deletions in the VL CDRs. In some embodiments, the variant has up to about 5 amino acid substitutions, additions, and/or deletions in the VL CDRs.

In some embodiments, the antibodies or antigen-binding fragments thereof that that can be used in methods provided herein comprise a light chain variable region (VL) comprising (1) a light chain CDR1 (VL CDR1) having the amino acid sequence of SEQ ID NO: 45; (2) a light chain CDR (VL CDR2) having the amino acid sequence of SEQ ID NO: 46; or (3) a light chain CDR3 (VL CDR3) having the amino acid sequence of SEQ ID NO: 47; or a variant thereof having up to about 3, about 5, about 8, about 10, about 12, or about 15 amino acid substitutions, additions, and/or deletions in the VL CDRs. In some embodiments, the variant has about 5 amino acid substitutions, additions, and/or deletions in the VL CDRs. In some embodiments, the antibodies or antigen-binding fragments thereof that specifically bind to CD74 comprise a VL comprising (1) a VL CDR1 having the amino acid sequence of SEQ ID NO: 45; (2) a VL CDR2 having the amino acid sequence of SEQ ID NO: 46; and (3) a VL CDR3 having the amino acid sequence of SEQ ID NO: 47; or a variant thereof having up to about 3, about 5, about 8, about 10, about 12, or about 15 amino acid substitutions, additions, and/or deletions in the VL CDRs. In some embodiments, the variant has up to about 5 amino acid substitutions, additions, and/or deletions in the VL CDRs.

In some embodiments, the antibodies or antigen-binding fragments thereof that that can be used in methods provided herein comprise a light chain variable region (VL) comprising (1) a light chain CDR1 (VL CDR1) having the amino acid sequence of SEQ ID NO: 55; (2) a light chain CDR (VL CDR2) having the amino acid sequence of SEQ ID NO: 56; or (3) a light chain CDR3 (VL CDR3) having the amino acid sequence of SEQ ID NO: 57; or a variant thereof having up to about 3, about 5, about 8, about 10, about 12, or about 15 amino acid substitutions, additions, and/or deletions in the VL CDRs. In some embodiments, the variant has about 5 amino acid substitutions, additions, and/or deletions in the VL CDRs. In some embodiments, the antibodies or antigen-binding fragments thereof that specifically bind to Aβ comprise a VL comprising (1) a VL CDR1 having the amino acid sequence of SEQ ID NO: 55; (2) a VL CDR2 having the amino acid sequence of SEQ ID NO: 56; and (3) a VL CDR3 having the amino acid sequence of SEQ ID NO: 57; or a variant thereof having up to about 3, about 5, about 8, about 10, about 12, or about 15 amino acid substitutions, additions, and/or deletions in the VL CDRs. In some embodiments, the variant has up to about 5 amino acid substitutions, additions, and/or deletions in the VL CDRs.

In some embodiments, the antibodies or antigen-binding fragments thereof that that can be used in methods provided herein comprise a light chain variable region (VL) comprising (1) a light chain CDR1 (VL CDR1) having the amino acid sequence of SEQ ID NO: 64 (2) a light chain CDR (VL CDR2) having the amino acid sequence of SEQ ID NO: 65; or (3) a light chain CDR3 (VL CDR3) having the amino acid sequence of SEQ ID NO: 66; or a variant thereof having up to about 3, about 5, about 8, about 10, about 12, or about 15 amino acid substitutions, additions, and/or deletions in the VL CDRs. In some embodiments, the variant has about 5 amino acid substitutions, additions, and/or deletions in the VL CDRs. In some embodiments, the antibodies or antigen-binding fragments thereof that specifically bind to Aβ comprise a VL comprising (1) a VL CDR 1 having the amino acid sequence of SEQ ID NO: 64; (2) a VL CDR2 having the amino acid sequence of SEQ ID NO: 65; and (3) a VL CDR3 having the amino acid sequence of SEQ ID NO: 66; or a variant thereof having up to about 3, about 5, about 8, about 10, about 12, or about 15 amino acid substitutions, additions, and/or deletions in the VL CDRs. In some embodiments, the variant has up to about 5 amino acid substitutions, additions, and/or deletions in the VL CDRs.

In some embodiments, the antibodies or antigen-binding fragments thereof that that can be used in methods provided herein comprise a light chain variable region (VL) comprising (1) a light chain CDR1 (VL CDR1) having the amino acid sequence of SEQ ID NO: 72 (2) a light chain CDR (VL CDR2) having the amino acid sequence of SEQ ID NO: 73; or (3) a light chain CDR3 (VL CDR3) having the amino acid sequence of SEQ ID NO: 74; or a variant thereof having up to about 3, about 5, about 8, about 10, about 12, or about 15 amino acid substitutions, additions, and/or deletions in the VL CDRs. In some embodiments, the variant has about 5 amino acid substitutions, additions, and/or deletions in the VL CDRs. In some embodiments, the antibodies or antigen-binding fragments thereof that specifically bind to Aβ comprise a VL comprising (1) a VL CDR 1 having the amino acid sequence of SEQ ID NO: 72; (2) a VL CDR2 having the amino acid sequence of SEQ ID NO: 73; and (3) a VL CDR3 having the amino acid sequence of SEQ ID NO: 74; or a variant thereof having up to about 3, about 5, about 8, about 10, about 12, or about 15 amino acid substitutions, additions, and/or deletions in the VL CDRs. In some embodiments, the variant has up to about 5 amino acid substitutions, additions, and/or deletions in the VL CDRs.

In some embodiments, the antibodies or antigen-binding fragments thereof that that can be used in methods provided herein comprise a light chain variable region (VL) comprising (1) a light chain CDR1 (VL CDR1) having the amino acid sequence of SEQ ID NO: 80 (2) a light chain CDR (VL CDR2) having the amino acid sequence of SEQ ID NO: 81; or (3) a light chain CDR3 (VL CDR3) having the amino acid sequence of SEQ ID NO: 82; or a variant thereof having up to about 3, about 5, about 8, about 10, about 12, or about 15 amino acid substitutions, additions, and/or deletions in the VL CDRs. In some embodiments, the variant has about 5 amino acid substitutions, additions, and/or deletions in the VL CDRs. In some embodiments, the antibodies or antigen-binding fragments thereof that specifically bind to Aβ comprise a VL comprising (1) a VL CDR 1 having the amino acid sequence of SEQ ID NO: 80; (2) a VL CDR2 having the amino acid sequence of SEQ ID NO: 81; and (3) a VL CDR3 having the amino acid sequence of SEQ ID NO: 82; or a variant thereof having up to about 3, about 5, about 8, about 10, about 12, or about 15 amino acid substitutions, additions, and/or deletions in the VL CDRs. In some embodiments, the variant has up to about 5 amino acid substitutions, additions, and/or deletions in the VL CDRs.

In some embodiments, the antibodies or antigen-binding fragments thereof that that can be used in methods provided herein comprise a light chain variable region (VL) comprising (1) a light chain CDR1 (VL CDR1) having the amino acid sequence of SEQ ID NO: 88 (2) a light chain CDR (VL CDR2) having the amino acid sequence of SEQ ID NO: 89; or (3) a light chain CDR3 (VL CDR3) having the amino acid sequence of SEQ ID NO: 90; or a variant thereof having up to about 3, about 5, about 8, about 10, about 12, or about 15 amino acid substitutions, additions, and/or deletions in the VL CDRs. In some embodiments, the variant has about 5 amino acid substitutions, additions, and/or deletions in the VL CDRs. In some embodiments, the antibodies or antigen-binding fragments thereof that specifically bind to Aβ comprise a VL comprising (1) a VL CDR 1 having the amino acid sequence of SEQ ID NO: 88; (2) a VL CDR2 having the amino acid sequence of SEQ ID NO: 89; and (3) a VL CDR3 having the amino acid sequence of SEQ ID NO: 90; or a variant thereof having up to about 3, about 5, about 8, about 10, about 12, or about 15 amino acid substitutions, additions, and/or deletions in the VL CDRs. In some embodiments, the variant has up to about 5 amino acid substitutions, additions, and/or deletions in the VL CDRs.

In some embodiments, provided herein are antibodies or antigen-binding fragments thereof that specifically bind to CD22 comprising a heavy chain variable region (VH) comprising (1) a heavy chain CDR1 (VH CDR1) having the amino acid sequence of SEQ ID NO: 4; (2) a heavy chain CDR2 (VH CDR2) having the amino acid sequence of SEQ ID NO: 5; or (3) a heavy chain CDR3 (VH CDR3) having the amino acid sequence of SEQ ID NO: 6; or a variant thereof having up to about 3, about 5, about 8, about 10, about 12, or about 15 amino acid substitutions, additions, and/or deletions in the VH CDRs. In some embodiments, the variant has up about 5 amino acid substitutions, additions, and/or deletions in the VH CDRs. In some embodiments, provided herein are antibodies or antigen-binding fragments thereof that specifically bind to CD22 comprising a VH comprising (1) a VH CDR1 having the amino acid sequence of SEQ ID NO: 4; (2) a VH CDR2 having the amino acid sequence of SEQ ID NO: 5; and (3) a VH CDR3 having the amino acid sequence of SEQ ID NO: 6; or a variant thereof having up to about 3, about 5, about 8, about 10, about 12, or about 15 amino acid substitutions, additions, and/or deletions in the VH CDRs. In some embodiments, the variant has up about 5 amino acid substitutions, additions, and/or deletions in the VH CDRs.

In some embodiments, provided herein are antibodies or antigen-binding fragments thereof that specifically bind to CD22 comprising a heavy chain variable region (VH) comprising (1) a heavy chain CDR1 (VH CDR1) having the amino acid sequence of SEQ ID NO: 14; (2) a heavy chain CDR2 (VH CDR2) having the amino acid sequence of SEQ ID NO: 15; or (3) a heavy chain CDR3 (VH CDR3) having the amino acid sequence of SEQ ID NO: 16; or a variant thereof having up to about 3, about 5, about 8, about 10, about 12, or about 15 amino acid substitutions, additions, and/or deletions in the VH CDRs. In some embodiments, the variant has up about 5 amino acid substitutions, additions, and/or deletions in the VH CDRs. In some embodiments, provided herein are antibodies or antigen-binding fragments thereof that specifically bind to CD22 comprising a VH comprising (1) a VH CDR1 having the amino acid sequence of SEQ ID NO: 14; (2) a VH CDR2 having the amino acid sequence of SEQ ID NO: 15; and (3) a VH CDR3 having the amino acid sequence of SEQ ID NO: 16; or a variant thereof having up to about 3, about 5, about 8, about 10, about 12, or about 15 amino acid substitutions, additions, and/or deletions in the VH CDRs. In some embodiments, the variant has up about 5 amino acid substitutions, additions, and/or deletions in the VH CDRs.

In some embodiments, provided herein are antibodies or antigen-binding fragments thereof that specifically bind to CD33 comprising a heavy chain variable region (VH) comprising (1) a heavy chain CDR1 (VH CDR1) having the amino acid sequence of SEQ ID NO: 24; (2) a heavy chain CDR2 (VH CDR2) having the amino acid sequence of SEQ ID NO: 25; or (3) a heavy chain CDR3 (VH CDR3) having the amino acid sequence of SEQ ID NO: 26; or a variant thereof having up to about 3, about 5, about 8, about 10, about 12, or about 15 amino acid substitutions, additions, and/or deletions in the VH CDRs. In some embodiments, the variant has up about 5 amino acid substitutions, additions, and/or deletions in the VH CDRs. In some embodiments, provided herein are antibodies or antigen-binding fragments thereof that specifically bind to CD33 comprising a VH comprising (1) a VH CDR1 having the amino acid sequence of SEQ ID NO: 24; (2) a VH CDR2 having the amino acid sequence of SEQ ID NO: 25; and (3) a VH CDR3 having the amino acid sequence of SEQ ID NO: 26; or a variant thereof having up to about 3, about 5, about 8, about 10, about 12, or about 15 amino acid substitutions, additions, and/or deletions in the VH CDRs. In some embodiments, the variant has up about 5 amino acid substitutions, additions, and/or deletions in the VH CDRs.

In some embodiments, provided herein are antibodies or antigen-binding fragments thereof that specifically bind to CD33 comprising a heavy chain variable region (VH) comprising (1) a heavy chain CDR1 (VH CDR1) having the amino acid sequence of SEQ ID NO: 32; (2) a heavy chain CDR2 (VH CDR2) having the amino acid sequence of SEQ ID NO: 33; or (3) a heavy chain CDR3 (VH CDR3) having the amino acid sequence of SEQ ID NO: 34; or a variant thereof having up to about 3, about 5, about 8, about 10, about 12, or about 15 amino acid substitutions, additions, and/or deletions in the VH CDRs. In some embodiments, the variant has up about 5 amino acid substitutions, additions, and/or deletions in the VH CDRs. In some embodiments, provided herein are antibodies or antigen-binding fragments thereof that specifically bind to CD33 comprising a VH comprising (1) a VH CDR1 having the amino acid sequence of SEQ ID NO: 32; (2) a VH CDR2 having the amino acid sequence of SEQ ID NO: 33; and (3) a VH CDR3 having the amino acid sequence of SEQ ID NO: 34; or a variant thereof having up to about 3, about 5, about 8, about 10, about 12, or about 15 amino acid substitutions, additions, and/or deletions in the VH CDRs. In some embodiments, the variant has up about 5 amino acid substitutions, additions, and/or deletions in the VH CDRs.

In some embodiments, provided herein are antibodies or antigen-binding fragments thereof that specifically bind to CD33 comprising a heavy chain variable region (VH) comprising (1) a heavy chain CDR1 (VH CDR1) having the amino acid sequence of SEQ ID NO: 40; (2) a heavy chain CDR2 (VH CDR2) having the amino acid sequence of SEQ ID NO: 41; or (3) a heavy chain CDR3 (VH CDR3) having the amino acid sequence of SEQ ID NO: 42; or a variant thereof having up to about 3, about 5, about 8, about 10, about 12, or about 15 amino acid substitutions, additions, and/or deletions in the VH CDRs. In some embodiments, the variant has up about 5 amino acid substitutions, additions, and/or deletions in the VH CDRs. In some embodiments, provided herein are antibodies or antigen-binding fragments thereof that specifically bind to CD33 comprising a VH comprising (1) a VH CDR1 having the amino acid sequence of SEQ ID NO: 40; (2) a VH CDR2 having the amino acid sequence of SEQ ID NO: 41; and (3) a VH CDR3 having the amino acid sequence of SEQ ID NO: 42; or a variant thereof having up to about 3, about 5, about 8, about 10, about 12, or about 15 amino acid substitutions, additions, and/or deletions in the VH CDRs. In some embodiments, the variant has up about 5 amino acid substitutions, additions, and/or deletions in the VH CDRs.

In some embodiments, provided herein are antibodies or antigen-binding fragments thereof that specifically bind to CD74 comprising a heavy chain variable region (VH) comprising (1) a heavy chain CDR1 (VH CDR1) having the amino acid sequence of SEQ ID NO: 48; (2) a heavy chain CDR2 (VH CDR2) having the amino acid sequence of SEQ ID NO: 49; or (3) a heavy chain CDR3 (VH CDR3) having the amino acid sequence of SEQ ID NO: 50; or a variant thereof having up to about 3, about 5, about 8, about 10, about 12, or about 15 amino acid substitutions, additions, and/or deletions in the VH CDRs. In some embodiments, the variant has up about 5 amino acid substitutions, additions, and/or deletions in the VH CDRs. In some embodiments, provided herein are antibodies or antigen-binding fragments thereof that specifically bind to CD74 comprising a VH comprising (1) a VH CDR1 having the amino acid sequence of SEQ ID NO: 48; (2) a VH CDR2 having the amino acid sequence of SEQ ID NO: 49; and (3) a VH CDR3 having the amino acid sequence of SEQ ID NO: 50; or a variant thereof having up to about 3, about 5, about 8, about 10, about 12, or about 15 amino acid substitutions, additions, and/or deletions in the VH CDRs. In some embodiments, the variant has up about 5 amino acid substitutions, additions, and/or deletions in the VH CDRs.

In some embodiments, provided herein are antibodies or antigen-binding fragments thereof that specifically bind to Aβ comprising a heavy chain variable region (VH) comprising (1) a heavy chain CDR1 (VH CDR1) having the amino acid sequence of SEQ ID NO: 58; (2) a heavy chain CDR2 (VH CDR2) having the amino acid sequence of SEQ ID NO: 59; or (3) a heavy chain CDR3 (VH CDR3) having the amino acid sequence of SEQ ID NO: 60; or a variant thereof having up to about 3, about 5, about 8, about 10, about 12, or about 15 amino acid substitutions, additions, and/or deletions in the VH CDRs. In some embodiments, the variant has up about 5 amino acid substitutions, additions, and/or deletions in the VH CDRs. In some embodiments, provided herein are antibodies or antigen-binding fragments thereof that specifically bind to Aβ comprising a VH comprising (1) a VH CDR1 having the amino acid sequence of SEQ ID NO: 58; (2) a VH CDR2 having the amino acid sequence of SEQ ID NO: 59; and (3) a VH CDR3 having the amino acid sequence of SEQ ID NO: 60; or a variant thereof having up to about 3, about 5, about 8, about 10, about 12, or about 15 amino acid substitutions, additions, and/or deletions in the VH CDRs. In some embodiments, the variant has up about 5 amino acid substitutions, additions, and/or deletions in the VH CDRs.

In some embodiments, provided herein are antibodies or antigen-binding fragments thereof that specifically bind to Aβ comprising a heavy chain variable region (VH) comprising (1) a heavy chain CDR1 (VH CDR1) having the amino acid sequence of SEQ ID NO: 67; (2) a heavy chain CDR2 (VH CDR2) having the amino acid sequence of SEQ ID NO: 68; or (3) a heavy chain CDR3 (VH CDR3) having the amino acid sequence of SEQ ID NO: 69; or a variant thereof having up to about 3, about 5, about 8, about 10, about 12, or about 15 amino acid substitutions, additions, and/or deletions in the VH CDRs. In some embodiments, the variant has up about 5 amino acid substitutions, additions, and/or deletions in the VH CDRs. In some embodiments, provided herein are antibodies or antigen-binding fragments thereof that specifically bind to Aβ comprising a VH comprising (1) a VH CDR1 having the amino acid sequence of SEQ ID NO: 67; (2) a VH CDR2 having the amino acid sequence of SEQ ID NO: 68; and (3) a VH CDR3 having the amino acid sequence of SEQ ID NO: 69; or a variant thereof having up to about 3, about 5, about 8, about 10, about 12, or about 15 amino acid substitutions, additions, and/or deletions in the VH CDRs. In some embodiments, the variant has up about 5 amino acid substitutions, additions, and/or deletions in the VH CDRs.

In some embodiments, provided herein are antibodies or antigen-binding fragments thereof that specifically bind to Aβ comprising a heavy chain variable region (VH) comprising (1) a heavy chain CDR1 (VH CDR1) having the amino acid sequence of SEQ ID NO: 75; (2) a heavy chain CDR2 (VH CDR2) having the amino acid sequence of SEQ ID NO: 76; or (3) a heavy chain CDR3 (VH CDR3) having the amino acid sequence of SEQ ID NO: 77; or a variant thereof having up to about 3, about 5, about 8, about 10, about 12, or about 15 amino acid substitutions, additions, and/or deletions in the VH CDRs. In some embodiments, the variant has up about 5 amino acid substitutions, additions, and/or deletions in the VH CDRs. In some embodiments, provided herein are antibodies or antigen-binding fragments thereof that specifically bind to Aβ comprising a VH comprising (1) a VH CDR1 having the amino acid sequence of SEQ ID NO: 75; (2) a VH CDR2 having the amino acid sequence of SEQ ID NO: 76; and (3) a VH CDR3 having the amino acid sequence of SEQ ID NO: 77; or a variant thereof having up to about 3, about 5, about 8, about 10, about 12, or about 15 amino acid substitutions, additions, and/or deletions in the VH CDRs. In some embodiments, the variant has up about 5 amino acid substitutions, additions, and/or deletions in the VH CDRs.

In some embodiments, provided herein are antibodies or antigen-binding fragments thereof that specifically bind to Aβ comprising a heavy chain variable region (VH) comprising (1) a heavy chain CDR1 (VH CDR1) having the amino acid sequence of SEQ ID NO: 83; (2) a heavy chain CDR2 (VH CDR2) having the amino acid sequence of SEQ ID NO: 84; or (3) a heavy chain CDR3 (VH CDR3) having the amino acid sequence of SEQ ID NO: 85; or a variant thereof having up to about 3, about 5, about 8, about 10, about 12, or about 15 amino acid substitutions, additions, and/or deletions in the VH CDRs. In some embodiments, the variant has up about 5 amino acid substitutions, additions, and/or deletions in the VH CDRs. In some embodiments, provided herein are antibodies or antigen-binding fragments thereof that specifically bind to Aβ comprising a VH comprising (1) a VH CDR1 having the amino acid sequence of SEQ ID NO: 83; (2) a VH CDR2 having the amino acid sequence of SEQ ID NO: 84; and (3) a VH CDR3 having the amino acid sequence of SEQ ID NO: 85; or a variant thereof having up to about 3, about 5, about 8, about 10, about 12, or about 15 amino acid substitutions, additions, and/or deletions in the VH CDRs. In some embodiments, the variant has up about 5 amino acid substitutions, additions, and/or deletions in the VH CDRs.

In some embodiments, provided herein are antibodies or antigen-binding fragments thereof that specifically bind to Aβ comprising a heavy chain variable region (VH) comprising (1) a heavy chain CDR1 (VH CDR1) having the amino acid sequence of SEQ ID NO: 91; (2) a heavy chain CDR2 (VH CDR2) having the amino acid sequence of SEQ ID NO: 92; or (3) a heavy chain CDR3 (VH CDR3) having the amino acid sequence of SEQ ID NO: 93; or a variant thereof having up to about 3, about 5, about 8, about 10, about 12, or about 15 amino acid substitutions, additions, and/or deletions in the VH CDRs. In some embodiments, the variant has up about 5 amino acid substitutions, additions, and/or deletions in the VH CDRs. In some embodiments, provided herein are antibodies or antigen-binding fragments thereof that specifically bind to Aβ comprising a VH comprising (1) a VH CDR1 having the amino acid sequence of SEQ ID NO: 91; (2) a VH CDR2 having the amino acid sequence of SEQ ID NO: 92; and (3) a VH CDR3 having the amino acid sequence of SEQ ID NO: 93; or a variant thereof having up to about 3, about 5, about 8, about 10, about 12, or about 15 amino acid substitutions, additions, and/or deletions in the VH CDRs. In some embodiments, the variant has up about 5 amino acid substitutions, additions, and/or deletions in the VH CDRs.

In some embodiments, provided herein are antibodies or antigen-binding fragments thereof that specifically bind to CD22, comprising (a) a VL comprising (1) a VL CDR1 having the amino acid sequence of SEQ ID NO: 1; (2) a VL CDR2 having the amino acid sequence of SEQ ID NO: 2; and (3) a VL CDR3 having the amino acid sequence of SEQ ID NO: 3; or a variant thereof having up to about 5 amino acid substitutions, additions, and/or deletions in the VL CDRs; and (b) a VH comprising (1) a VH CDR1 having the amino acid sequence of SEQ ID NO: 4; (2) a VH CDR2 having the amino acid sequence of SEQ ID NO: 5; and (3) a VH CDR3 having the amino acid sequence of SEQ ID NO: 6; or a variant thereof having up to about 5 amino acid substitutions, additions, and/or deletions in the VH CDRs.

In some embodiments, provided herein are antibodies or antigen-binding fragments thereof that specifically bind to CD22, comprising (a) a VL comprising (1) a VL CDR1 having the amino acid sequence of SEQ ID NO: 11; (2) a VL CDR2 having the amino acid sequence of SEQ ID NO: 12; and (3) a VL CDR3 having the amino acid sequence of SEQ ID NO: 13; or a variant thereof having up to about 5 amino acid substitutions, additions, and/or deletions in the VL CDRs; and (b) a VH comprising (1) a VH CDR1 having the amino acid sequence of SEQ ID NO: 14; (2) a VH CDR2 having the amino acid sequence of SEQ ID NO: 15; and (3) a VH CDR3 having the amino acid sequence of SEQ ID NO: 16; or a variant thereof having up to about 5 amino acid substitutions, additions, and/or deletions in the VH CDRs.

In some embodiments, provided herein are antibodies or antigen-binding fragments thereof that specifically bind to CD33 comprising (a) a VL comprising (1) a VL CDR1 having the amino acid sequence of SEQ ID NO: 21; (2) a VL CDR2 having the amino acid sequence of SEQ ID NO: 22; and (3) a VL CDR3 having the amino acid sequence of SEQ ID NO: 23; or a variant thereof having up to about 5 amino acid substitutions, additions, and/or deletions in the VL CDRs; and (b) a VH comprising (1) a VH CDR1 having the amino acid sequence of SEQ ID NO: 24; (2) a VH CDR2 having the amino acid sequence of SEQ ID NO: 25 and (3) a VH CDR3 having the amino acid sequence of SEQ ID NO: 26; or a variant thereof having up to about 5 amino acid substitutions, additions, and/or deletions in the VH CDRs.

In some embodiments, provided herein are antibodies or antigen-binding fragments thereof that specifically bind to CD33 comprising (a) a VL comprising (1) a VL CDR1 having the amino acid sequence of SEQ ID NO: 29; (2) a VL CDR2 having the amino acid sequence of SEQ ID NO: 30; and (3) a VL CDR3 having the amino acid sequence of SEQ ID NO: 31; or a variant thereof having up to about 5 amino acid substitutions, additions, and/or deletions in the VL CDRs; and (b) a VH comprising (1) a VH CDR1 having the amino acid sequence of SEQ ID NO: 32; (2) a VH CDR2 having the amino acid sequence of SEQ ID NO: 33 and (3) a VH CDR3 having the amino acid sequence of SEQ ID NO: 34; or a variant thereof having up to about 5 amino acid substitutions, additions, and/or deletions in the VH CDRs.

In some embodiments, provided herein are antibodies or antigen-binding fragments thereof that specifically bind to CD33 comprising (a) a VL comprising (1) a VL CDR1 having the amino acid sequence of SEQ ID NO: 37; (2) a VL CDR2 having the amino acid sequence of SEQ ID NO: 38; and (3) a VL CDR3 having the amino acid sequence of SEQ ID NO: 39; or a variant thereof having up to about 5 amino acid substitutions, additions, and/or deletions in the VL CDRs; and (b) a VH comprising (1) a VH CDR1 having the amino acid sequence of SEQ ID NO: 40; (2) a VH CDR2 having the amino acid sequence of SEQ ID NO: 41 and (3) a VH CDR3 having the amino acid sequence of SEQ ID NO: 42; or a variant thereof having up to about 5 amino acid substitutions, additions, and/or deletions in the VH CDRs.

In some embodiments, provided herein are antibodies or antigen-binding fragments thereof that specifically bind to CD33 comprising (a) a VL comprising (1) a VL CDR1 having the amino acid sequence of SEQ ID NO: 45; (2) a VL CDR2 having the amino acid sequence of SEQ ID NO: 46; and (3) a VL CDR3 having the amino acid sequence of SEQ ID NO: 47; or a variant thereof having up to about 5 amino acid substitutions, additions, and/or deletions in the VL CDRs; and (b) a VH comprising (1) a VH CDR1 having the amino acid sequence of SEQ ID NO: 48; (2) a VH CDR2 having the amino acid sequence of SEQ ID NO: 49 and (3) a VH CDR3 having the amino acid sequence of SEQ ID NO: 50; or a variant thereof having up to about 5 amino acid substitutions, additions, and/or deletions in the VH CDRs.

In some embodiments, provided herein are antibodies or antigen-binding fragments thereof that specifically bind to CD33 comprising (a) a VL comprising (1) a VL CDR1 having the amino acid sequence of SEQ ID NO: 55; (2) a VL CDR2 having the amino acid sequence of SEQ ID NO: 56; and (3) a VL CDR3 having the amino acid sequence of SEQ ID NO: 57; or a variant thereof having up to about 5 amino acid substitutions, additions, and/or deletions in the VL CDRs; and (b) a VH comprising (1) a VH CDR1 having the amino acid sequence of SEQ ID NO: 58; (2) a VH CDR2 having the amino acid sequence of SEQ ID NO: 59 and (3) a VH CDR3 having the amino acid sequence of SEQ ID NO: 60; or a variant thereof having up to about 5 amino acid substitutions, additions, and/or deletions in the VH CDRs.

In some embodiments, provided herein are antibodies or antigen-binding fragments thereof that specifically bind to CD33 comprising (a) a VL comprising (1) a VL CDR1 having the amino acid sequence of SEQ ID NO: 64; (2) a VL CDR2 having the amino acid sequence of SEQ ID NO: 65; and (3) a VL CDR3 having the amino acid sequence of SEQ ID NO: 66; or a variant thereof having up to about 5 amino acid substitutions, additions, and/or deletions in the VL CDRs; and (b) a VH comprising (1) a VH CDR1 having the amino acid sequence of SEQ ID NO: 67; (2) a VH CDR2 having the amino acid sequence of SEQ ID NO: 68 and (3) a VH CDR3 having the amino acid sequence of SEQ ID NO: 69; or a variant thereof having up to about 5 amino acid substitutions, additions, and/or deletions in the VH CDRs.

In some embodiments, provided herein are antibodies or antigen-binding fragments thereof that specifically bind to CD33 comprising (a) a VL comprising (1) a VL CDR1 having the amino acid sequence of SEQ ID NO: 72; (2) a VL CDR2 having the amino acid sequence of SEQ ID NO: 73; and (3) a VL CDR3 having the amino acid sequence of SEQ ID NO: 74; or a variant thereof having up to about 5 amino acid substitutions, additions, and/or deletions in the VL CDRs; and (b) a VH comprising (1) a VH CDR1 having the amino acid sequence of SEQ ID NO: 75; (2) a VH CDR2 having the amino acid sequence of SEQ ID NO: 76 and (3) a VH CDR3 having the amino acid sequence of SEQ ID NO: 77; or a variant thereof having up to about 5 amino acid substitutions, additions, and/or deletions in the VH CDRs.

In some embodiments, provided herein are antibodies or antigen-binding fragments thereof that specifically bind to CD33 comprising (a) a VL comprising (1) a VL CDR1 having the amino acid sequence of SEQ ID NO: 80; (2) a VL CDR2 having the amino acid sequence of SEQ ID NO: 81; and (3) a VL CDR3 having the amino acid sequence of SEQ ID NO: 82; or a variant thereof having up to about 5 amino acid substitutions, additions, and/or deletions in the VL CDRs; and (b) a VH comprising (1) a VH CDR1 having the amino acid sequence of SEQ ID NO: 83; (2) a VH CDR2 having the amino acid sequence of SEQ ID NO: 84 and (3) a VH CDR3 having the amino acid sequence of SEQ ID NO: 85; or a variant thereof having up to about 5 amino acid substitutions, additions, and/or deletions in the VH CDRs.

In some embodiments, provided herein are antibodies or antigen-binding fragments thereof that specifically bind to CD33 comprising (a) a VL comprising (1) a VL CDR1 having the amino acid sequence of SEQ ID NO: 88; (2) a VL CDR2 having the amino acid sequence of SEQ ID NO: 89; and (3) a VL CDR3 having the amino acid sequence of SEQ ID NO: 90; or a variant thereof having up to about 5 amino acid substitutions, additions, and/or deletions in the VL CDRs; and (b) a VH comprising (1) a VH CDR1 having the amino acid sequence of SEQ ID NO: 91; (2) a VH CDR2 having the amino acid sequence of SEQ ID NO: 92 and (3) a VH CDR3 having the amino acid sequence of SEQ ID NO: 93; or a variant thereof having up to about 5 amino acid substitutions, additions, and/or deletions in the VH CDRs.

It is well known in the art that VH CDR3 and VL CDR3 domains play an important role in the binding specificity/affinity of an antibody for an antigen. Accordingly, in some embodiments, the anti-CD22, anti-CD33 and anti-CD74 bispecific antibodies or antigen-binding fragments thereof that can be used in methods disclosed herein can have the appropriate association/dissociation kinetics with the respective receptors/antigens and have the VH CDR3 and VL CDR3 that are structurally identical to or related to the corresponding antibodies. For illustrative purpose, for example, in the case of the anti-CD22 antibody SM03, a consensus motif for the SM03 VL CDR3 comprising the amino acid sequence: Q-Q-G-N-T-L-P-W-T (SEQ ID NO: 3) can be modified by substituting one or more of the amino acid(s) to adjust the antibody affinity without changing its binding specificity, or alternatively be replaced by the VL CDR3 of an irrelevant human antibody that exhibits sufficient similarities to the SM03 VL CDR3 using criteria as described in Chinese Pat. No. ZL200880024788.2, which is incorporated herewith by reference. Similarly, a consensus motif for the SM03 VH CDR3 comprising the amino acid sequence: H-S-G-Y-G-S-S-Y-G-V-L-F-A-Y (SEQ ID NO: 6) can be modified by substituting one or more of the amino acid(s) to adjust the antibody affinity without changing its binding specificity, or alternatively be replaced by the VH CDR3 of an irrelevant human antibody that exhibits sufficient similarities to the SM03 VH CDR3 using criteria as described in Chinese Pat. No. ZL200880024788.2, which is incorporated herewith by reference. The skilled artisan would appreciate that certain substitution(s) of amino acids within the CDR3 domains would not change the epitope specificity of the antibody, in particular substitutions with conservative amino acids. As such, in some embodiments, the CDR3 of the antibodies or antigen-binding fragments provided herein (e.g., SM03, SM06, LL2, hLL2, Gemtuzumab, HuMy9-6, Lintuzumab cLL1, hLL1, Aducanumab, BAN2401, Gantenerumab, Crenezumab, AD38) can be replaced with the CDR3 from a human or primate antibody that (1) is identical in the number of residues and exhibits 50% or higher sequence homology to the CDR3 of the corresponding antibody, (2) contains at least one, preferably more, aromatic residue(s) that is (arc) identical or conservatively similar to the residue(s) at corresponding position(s) in the CDR3 of the corresponding antibody, (3) contains at least one, preferably more, charged residue(s) that is (are) identical or conservatively similar to the residue(s) at corresponding position(s) in the CDR3 of the corresponding antibody, and/or (4) contains at least one, preferably more, amino acid residue(s) that is/are identical or conservatively similar to the residue(s) at corresponding position(s) in the CDR3 of the corresponding antibody at positions that are known to be important for maintaining the binding site structure/contacts of the corresponding antibody as determined by crystal structure and/or computer database analysis (see for example Chinese Pat. No. ZL200880024788.2, which is incorporated herewith by reference). In some embodiments, no more than one to five conservative amino acid substitutions are made with the VL and/or VH CDR3 domains, or VL and/or VH CDR3 of the corresponding antibody from irrelevant primate or human antibodies containing no more than one to five conservatively similar residues are used to replace the VL and/or VH CDR3 of the corresponding antibody. In some embodiments, no more than one to three conservative amino acid substitutions are made within the VL and/or VH CDR3 domains, or VL and/or VH CDR3 of the corresponding antibody from irrelevant primate or human antibodies containing no more than one to three conservatively similar residues is used to replace the VL and/or VH CDR3 of of the corresponding antibody.

In some embodiments, provided herein are antibodies or antigen-binding fragments thereof that specifically bind to CD22 comprising a VL and a VH, wherein the VL and VH have the amino acid sequences of SEQ ID NOs: 7 and 8, respectively. In some embodiments, the VL and VH have the amino acid sequences of SEQ ID NOs: 9 and 10, respectively.

In some embodiments, provided herein are antibodies or antigen-binding fragments thereof that specifically bind to CD22 comprising a VL and a VH, wherein the VL and VH have the amino acid sequences of SEQ ID NOs: 7 and 18, respectively. In some embodiments, the VL and VH have the amino acid sequences of SEQ ID NOs: 19 and 20, respectively.

In some embodiments, provided herein are antibodies or antigen-binding fragments thereof that specifically bind to CD33 comprising a VL and a VH, wherein the VL and VH have the amino acid sequences of SEQ ID NOs: 27 and 28, respectively.

In some embodiments, provided herein are antibodies or antigen-binding fragments thereof that specifically bind to CD33 comprising a VL and a VH, wherein the VL and VH have the amino acid sequences of SEQ ID NOs: 35 and 36, respectively.

In some embodiments, provided herein are antibodies or antigen-binding fragments thereof that specifically bind to CD33 comprising a VL and a VH, wherein the VL and VH have the amino acid sequences of SEQ ID NOs: 43 and 44, respectively.

In some embodiments, provided herein are antibodies or antigen-binding fragments thereof that specifically bind to CD74 comprising a VL and a VH, wherein the VL and VH have the amino acid sequences of SEQ ID NOs: 51 and 52, respectively. In some embodiments, the VL and VH have the amino acid sequences of SEQ ID NOs: 53 and 54, respectively.

In some embodiments, provided herein are antibodies or antigen-binding fragments thereof that specifically bind to Aβ comprising a VL and a VH, wherein the VL and VH have the amino acid sequences of SEQ ID NOs: 61 and 62, respectively. In some embodiments, the VL and VH have the amino acid sequences of SEQ ID NOs: 61 and 63, respectively.

In some embodiments, provided herein are antibodies or antigen-binding fragments thereof that specifically bind to Aβ comprising a VL and a VH, wherein the VL and VH have the amino acid sequences of SEQ ID NOs: 70 and 71, respectively.

In some embodiments, provided herein are antibodies or antigen-binding fragments thereof that specifically bind to Aβ comprising a VL and a VH, wherein the VL and VH have the amino acid sequences of SEQ ID NOs: 78 and 79, respectively.

In some embodiments, provided herein are antibodies or antigen-binding fragments thereof that specifically bind to Aβ comprising a VL and a VH, wherein the VL and VH have the amino acid sequences of SEQ ID NOs: 86 and 87, respectively.

In some embodiments, provided herein are antibodies or antigen-binding fragments thereof that specifically bind to Aβ comprising a VL and a VH, wherein the VL and VH have the amino acid sequences of SEQ ID NOs: 94 and 95, respectively.

The anti-CD22, anti-CD33, anti-CD74 and anti-Aβ antibodies or antigen-binding fragments thereof can comprise a combination of any VL disclosed herein and any VH disclosed herein. In some embodiments, the VL and VH are connected by a linker. The linker can be a flexible linker or a rigid linker. In some embodiments, the linker is a flexible linker. In some embodiments, the linker has the amino acid sequence of (GGGGS) n, n=3, 4, or 5 (SEQ ID NO: 142). In some embodiments, the linker has the amino acid sequence of GSAGSAAGSGEF (SEQ ID NO: 161). In some embodiments, the linker has the amino acid sequence of KESGSVSSEQLAQFRSLD (SEQ ID NO: 162). In some embodiments, the linker has the amino acid sequence of EGKSSGSGSESKS (SEQ ID NO: 163). In some embodiments, the linker has the amino acid sequence of SSGNSNANSRGPSFSSGLVPLSLRGSH (SEQ ID NO: 164). In some embodiments, the linker has the amino acid sequence of GGGGGG (SEQ ID NO: 165). In some embodiments, the linker is a rigid linker. In some embodiments, the linker has the amino acid sequence of (EAAAK) n, n=3, 4, or 5 (SEQ ID NO: 143). In some embodiments, the linker has the amino acid sequence of (PA) nP, n=1, 2, 3, 4, or 5 (SEQ ID NO: 144 and SEQ ID NOs: 166-168).

In some embodiments, provided herein are antibodies or antigen-binding fragments thereof that specifically bind to CD22 comprising (a) a VL comprising VL CDRs 1, 2, and 3 from a VL having the amino acid sequence of SEQ ID NO: 7 or 9; and/or (b) a VH comprising VH CDRs 1, 2, and 3 from a VH having the amino acid sequence of SEQ ID NO: 8 or 10. In some embodiments, provided herein are antibodies or antigen-binding fragments thereof that specifically bind to CD22 comprising (a) a VL comprising VL CDRs 1, 2, and 3 from a VL having the amino acid sequence of SEQ ID NO: 9; and/or (b) a VH comprising VH CDRs 1, 2, and 3 from a VH having the amino acid sequence of SEQ ID NO: 10.

In some embodiments, provided herein are antibodies or antigen-binding fragments thereof that specifically bind to CD22 comprising (a) a VL comprising VL CDRs 1, 2, and 3 from a VL having the amino acid sequence of SEQ ID NO: 17 or 19; and/or (b) a VH comprising VH CDRs 1, 2, and 3 from a VH having the amino acid sequence of SEQ ID NO: 18 or 20. In some embodiments, provided herein are antibodies or antigen-binding fragments thereof that specifically bind to CD22 comprising (a) a VL comprising VL CDRs 1, 2, and 3 from a VL having the amino acid sequence of SEQ ID NO: 19; and/or (b) a VH comprising VH CDRs 1, 2, and 3 from a VH having the amino acid sequence of SEQ ID NO: 20.

In some embodiments, provided herein are antibodies or antigen-binding fragments thereof that specifically bind to CD33 comprising (a) a VL comprising VL CDRs 1, 2, and 3 from a VL having the amino acid sequence of SEQ ID NO: 27; and/or (b) a VH comprising VH CDRs 1, 2, and 3 from a VH having the amino acid sequence of SEQ ID NO: 28.

In some embodiments, provided herein are antibodies or antigen-binding fragments thereof that specifically bind to CD33 comprising (a) a VL comprising VL CDRs 1, 2, and 3 from a VL having the amino acid sequence of SEQ ID NO: 35; and/or (b) a VH comprising VH CDRs 1, 2, and 3 from a VH having the amino acid sequence of SEQ ID NO: 36.

In some embodiments, provided herein are antibodies or antigen-binding fragments thereof that specifically bind to CD33 comprising (a) a VL comprising VL CDRs 1, 2, and 3 from a VL having the amino acid sequence of SEQ ID NO: 43; and/or (b) a VH comprising VH CDRs 1, 2, and 3 from a VH having the amino acid sequence of SEQ ID NO: 44.

In some embodiments, provided herein are antibodies or antigen-binding fragments thereof that specifically bind to CD74 comprising (a) a VL comprising VL CDRs 1, 2, and 3 from a VL having the amino acid sequence of SEQ ID NO: 51 or 53; and/or (b) a VH comprising VH CDRs 1, 2, and 3 from a VH having the amino acid sequence of SEQ ID NO: 52 or 54. In some embodiments, provided herein are antibodies or antigen-binding fragments thereof that specifically bind to CD74 comprising (a) a VL comprising VL CDRs 1, 2, and 3 from a VL having the amino acid sequence of SEQ ID NO: 53; and/or (b) a VH comprising VH CDRs 1, 2, and 3 from a VH having the amino acid sequence of SEQ ID NO: 54.

In some embodiments, provided herein are antibodies or antigen-binding fragments thereof that specifically bind to Aβ comprising (a) a VL comprising VL CDRs 1, 2, and 3 from a VL having the amino acid sequence of SEQ ID NO: 61; and/or (b) a VH comprising VH CDRs 1, 2, and 3 from a VH having the amino acid sequence of SEQ ID NO: 62 or 63. In some embodiments, provided herein are antibodies or antigen-binding fragments thereof that specifically bind to Aβ comprising (a) a VL comprising VL CDRs 1, 2, and 3 from a VL having the amino acid sequence of SEQ ID NO: 61; and/or (b) a VH comprising VH CDRs 1, 2, and 3 from a VH having the amino acid sequence of SEQ ID NO: 63.

In some embodiments, provided herein are antibodies or antigen-binding fragments thereof that specifically bind to Aβ comprising (a) a VL comprising VL CDRs 1, 2, and 3 from a VL having the amino acid sequence of SEQ ID NO: 70; and/or (b) a VH comprising VH CDRs 1, 2, and 3 from a VH having the amino acid sequence of SEQ ID NO: 71.

In some embodiments, provided herein are antibodies or antigen-binding fragments thereof that specifically bind to Aβ comprising (a) a VL comprising VL CDRs 1, 2, and 3 from a VL having the amino acid sequence of SEQ ID NO: 78; and/or (b) a VH comprising VH CDRs 1, 2, and 3 from a VH having the amino acid sequence of SEQ ID NO: 79.

In some embodiments, provided herein are antibodies or antigen-binding fragments thereof that specifically bind to Aβ comprising (a) a VL comprising VL CDRs 1, 2, and 3 from a VL having the amino acid sequence of SEQ ID NO: 86; and/or (b) a VH comprising VH CDRs 1, 2, and 3 from a VH having the amino acid sequence of SEQ ID NO: 87.

In some embodiments, provided herein are antibodies or antigen-binding fragments thereof that specifically bind to Aβ comprising (a) a VL comprising VL CDRs 1, 2, and 3 from a VL having the amino acid sequence of SEQ ID NO: 94; and/or (b) a VH comprising VH CDRs 1, 2, and 3 from a VH having the amino acid sequence of SEQ ID NO: 95.

In some embodiments, provided herein are antibodies or antigen-binding fragments thereof that specifically bind to CD22 comprising a VL, wherein the VL comprises VL CDRs 1, 2, and 3 from a VL having the amino acid sequence of SEQ ID NO: 7. In some embodiments, the VL comprises VL CDRs 1, 2, and 3 from a VL having the amino acid sequence of SEQ ID NO: 9.

In some embodiments, provided herein are antibodies or antigen-binding fragments thereof that specifically bind to CD22 comprising a VH, wherein the VH comprises VH CDRs 1, 2, and 3 from a VH having the amino acid sequence of SEQ ID NO: 8. In some embodiments, the VH comprises VH CDRs 1, 2, and 3 from a VH having the amino acid sequence of SEQ ID NO: 10.

In some embodiments, provided herein are antibodies or antigen-binding fragments thereof that specifically bind to CD22 comprising a VL and a VH, wherein the VL comprises VL CDR1, CDR2, and CDR3 from a VL having the amino acid sequence of SEQ ID NO: 7, and the VH comprises VH CDR1, CDR2, and CDR3 from a VH having the amino acid sequence of SEQ ID NO: 8. In some embodiments, the VL comprises VL CDR1, CDR2, and CDR3 from a VL having the amino acid sequence of SEQ ID NO: 9, and the VH comprises VH CDR1, CDR2, and CDR3 from a VH having the amino acid sequence of SEQ ID NO: 10.

In some embodiments, the anti-CD22 antibody or antigen-binding fragment thereof provided herein is the antibody designated as SM03. In some embodiments, the anti-CD22 antibody or antigen-binding fragment thereof provided herein has a VL from SM03. In some embodiments, the anti-CD22 antibody or antigen-binding fragment thereof provided herein has a VH from SM03. The anti-CD22 antibody or antigen-binding fragment thereof provided herein can have both a VL and a VH from SM03. In some embodiments, the anti-CD22 antibody or antigen-binding fragment thereof provided herein has a VL that comprises VL CDRs 1, 2, and 3 from the VL from SM03. In some embodiments, the anti-CD22 antibody or antigen-binding fragment thereof provided herein has a VH that comprises VH CDRs 1, 2, and 3 from the VH from SM03. The anti-CD22 antibody or antigen-binding fragment thereof provided herein can have a VL comprising VL CDRs 1, 2, and 3 and a VH comprising VH CDRs 1, 2, and 3 from the VL and VH of SM03, respectively. In some embodiments, the anti-CD22 antibody or antigen-binding fragment thereof provided herein is a variant of SM03. The SM03 variant can have a VL that is a variant of the VL of SM03 having up to about 5 amino acid substitutions, additions, and/or deletions in SEQ ID NO: 7. The SM03 variant can have a VH that is a variant of the VH of SM03 having up to about 5 amino acid substitutions, additions, and/or deletions in SEQ ID NO: 8. The amino acid substitutions, additions, and/or deletions can be in the VH CDRs or VL CDRs. In some embodiments, the amino acid substitutions, additions, and/or deletions are not in the CDRs. In some embodiments, the variant of SM03 has up to about 5 conservative amino acid substitutions. In some embodiments, the variant of SM03 has up to 3 conservative amino acid substitutions.

In some embodiments, the anti-CD22 antibody or antigen-binding fragment thereof provided herein is the antibody designated as SM06. In some embodiments, the anti-CD22 antibody or antigen-binding fragment thereof provided herein has a VL from SM06. In some embodiments, the anti-CD22 antibody or antigen-binding fragment thereof provided herein has a VH from SM06. The anti-CD22 antibody or antigen-binding fragment thereof provided herein can have both a VL and a VH from SM06. In some embodiments, the anti-CD22 antibody or antigen-binding fragment thereof provided herein has a VL that comprises VL CDRs 1, 2, and 3 from the VL from SM06. In some embodiments, the anti-CD22 antibody or antigen-binding fragment thereof provided herein has a VH that comprises VH CDRs 1, 2, and 3 from the VH from SM06. The anti-CD22 antibody or antigen-binding fragment thereof provided herein can have a VL comprising VL CDRs 1, 2, and 3 and a VH comprising VH CDRs 1, 2, and 3 from the VL and VH of SM06, respectively. In some embodiments, the anti-CD22 antibody or antigen-binding fragment thereof provided herein is a variant of SM03. The SM06 variant can have a VL that is a variant of the VL of SM06 having up to about 5 amino acid substitutions, additions, and/or deletions in SEQ ID NO: 9. The SM06 variant can have a VH that is a variant of the VH of SM06 having up to about 5 amino acid substitutions, additions, and/or deletions in SEQ ID NO: 10. The amino acid substitutions, additions, and/or deletions can be in the VH CDRs or VL CDRs. In some embodiments, the amino acid substitutions, additions, and/or deletions are not in the CDRs. In some embodiments, the variant of SM06 has up to about 5 conservative amino acid substitutions. In some embodiments, the variant of SM06 has up to 3 conservative amino acid substitutions.

In some embodiments, the anti-CD22 antibody or antigen-binding fragment thereof provided herein is the antibody designated as hLL2. In some embodiments, the anti-CD22 antibody or antigen-binding fragment thereof provided herein has a VL from hLL2. In some embodiments, the anti-CD22 antibody or antigen-binding fragment thereof provided herein has a VH from hLL2. The anti-CD22 antibody or antigen-binding fragment thereof provided herein can have both a VL and a VH from hLL2. In some embodiments, the anti-CD22 antibody or antigen-binding fragment thereof provided herein has a VL that comprises VL CDRs 1, 2, and 3 from the VL from hLL2. In some embodiments, the anti-CD22 antibody or antigen-binding fragment thereof provided herein has a VH that comprises VH CDRs 1, 2, and 3 from the VH from hLL2. The anti-CD22 antibody or antigen-binding fragment thereof provided herein can have a VL comprising VL CDRs 1, 2, and 3 and a VH comprising VH CDRs 1, 2, and 3 from the VL and VH of hLL2, respectively. In some embodiments, the anti-CD22 antibody or antigen-binding fragment thereof provided herein is a variant of hLL2. The hLL2 variant can have a VL that is a variant of the VL of hLL2 having up to about 5 amino acid substitutions, additions, and/or deletions in SEQ ID NO: 19. The hLL2 variant can have a VH that is a variant of the VH of hLL2 having up to about 5 amino acid substitutions, additions, and/or deletions in SEQ ID NO: 20. The amino acid substitutions, additions, and/or deletions can be in the VH CDRs or VL CDRs. In some embodiments, the amino acid substitutions, additions, and/or deletions are not in the CDRs. In some embodiments, the variant of hLL2 has up to about 5 conservative amino acid substitutions. In some embodiments, the variant of hLL2 has up to 3 conservative amino acid substitutions.

In some embodiments, the anti-CD33 antibody or antigen-binding fragment thereof provided herein is the antibody designated as Gemtuzumab. In some embodiments, the anti-CD33 antibody or antigen-binding fragment thereof provided herein has a VL from Gemtuzumab. In some embodiments, the anti-CD33 antibody or antigen-binding fragment thereof provided herein has a VH from Gemtuzumab. The anti-CD33 antibody or antigen-binding fragment thereof provided herein can have both a VL and a VH from Gemtuzumab. In some embodiments, the anti-CD33 antibody or antigen-binding fragment thereof provided herein has a VL that comprises VL CDRs 1, 2, and 3 from the VL from Gemtuzumab. In some embodiments, the anti-CD33 antibody or antigen-binding fragment thereof provided herein has a VH that comprises VH CDRs 1, 2, and 3 from the VH from Gemtuzumab. The anti-CD33 antibody or antigen-binding fragment thereof provided herein can have a VL comprising VL CDRs 1, 2, and 3 and a VH comprising VH CDRs 1, 2, and 3 from the VL and VH of Gemtuzumab, respectively. In some embodiments, the anti-CD33 antibody or antigen-binding fragment thereof provided herein is a variant of Gemtuzumab. The Gemtuzumab variant can have a VL that is a variant of the VL of Gemtuzumab having up to about 5 amino acid substitutions, additions, and/or deletions in SEQ ID NO: 27. The Gemtuzumab variant can have a VH that is a variant of the VH of Gemtuzumab having up to about 5 amino acid substitutions, additions, and/or deletions in SEQ ID NO: 28. The amino acid substitutions, additions, and/or deletions can be in the VH CDRs or VL CDRs. In some embodiments, the amino acid substitutions, additions, and/or deletions are not in the CDRs. In some embodiments, the variant of Gemtuzumab has up to about 5 conservative amino acid substitutions. In some embodiments, the variant of Gemtuzumab has up to 3 conservative amino acid substitutions.

In some embodiments, the anti-CD33 antibody or antigen-binding fragment thereof provided herein is the antibody designated as HuMy9-6. In some embodiments, the anti-CD33 antibody or antigen-binding fragment thereof provided herein has a VL from HuMy9-6. In some embodiments, the anti-CD33 antibody or antigen-binding fragment thereof provided herein has a VH from HuMy9-6. The anti-CD33 antibody or antigen-binding fragment thereof provided herein can have both a VL and a VH from HuMy9-6. In some embodiments, the anti-CD33 antibody or antigen-binding fragment thereof provided herein has a VL that comprises VL CDRs 1, 2, and 3 from the VL from HuMy9-6. In some embodiments, the anti-CD33 antibody or antigen-binding fragment thereof provided herein has a VH that comprises VH CDRs 1, 2, and 3 from the VH from HuMy9-6. The anti-CD33 antibody or antigen-binding fragment thereof provided herein can have a VL comprising VL CDRs 1, 2, and 3 and a VH comprising VH CDRs 1, 2, and 3 from the VL and VH of HuMy9-6, respectively. In some embodiments, the anti-CD33 antibody or antigen-binding fragment thereof provided herein is a variant of HuMy9-6. The HuMy9-6 variant can have a VL that is a variant of the VL of HuMy9-6 having up to about 5 amino acid substitutions, additions, and/or deletions in SEQ ID NO: 35. The HuMy9-6 variant can have a VH that is a variant of the VH of HuMy9-6 having up to about 5 amino acid substitutions, additions, and/or deletions in SEQ ID NO: 36. The amino acid substitutions, additions, and/or deletions can be in the VH CDRs or VL CDRs. In some embodiments, the amino acid substitutions, additions, and/or deletions are not in the CDRs. In some embodiments, the variant of HuMy9-6 has up to about 5 conservative amino acid substitutions. In some embodiments, the variant of HuMy9-6 has up to 3 conservative amino acid substitutions.

In some embodiments, the anti-CD33 antibody or antigen-binding fragment thereof provided herein is the antibody designated as Lintuzumab. In some embodiments, the anti-CD33 antibody or antigen-binding fragment thereof provided herein has a VL from Lintuzumab. In some embodiments, the anti-CD33 antibody or antigen-binding fragment thereof provided herein has a VH from Lintuzumab. The anti-CD33 antibody or antigen-binding fragment thereof provided herein can have both a VL and a VH from Lintuzumab. In some embodiments, the anti-CD33 antibody or antigen-binding fragment thereof provided herein has a VL that comprises VL CDRs 1, 2, and 3 from the VL from Lintuzumab. In some embodiments, the anti-CD33 antibody or antigen-binding fragment thereof provided herein has a VH that comprises VH CDRs 1, 2, and 3 from the VH from Lintuzumab. The anti-CD33 antibody or antigen-binding fragment thereof provided herein can have a VL comprising VL CDRs 1, 2, and 3 and a VH comprising VH CDRs 1, 2, and 3 from the VL and VH of Lintuzumab, respectively. In some embodiments, the anti-CD33 antibody or antigen-binding fragment thereof provided herein is a variant of Lintuzumab. The Lintuzumab variant can have a VL that is a variant of the VL of Lintuzumab having up to about 5 amino acid substitutions, additions, and/or deletions in SEQ ID NO: 43. The Lintuzumab variant can have a VH that is a variant of the VH of Lintuzumab having up to about 5 amino acid substitutions, additions, and/or deletions in SEQ ID NO: 44. The amino acid substitutions, additions, and/or deletions can be in the VH CDRs or VL CDRs. In some embodiments, the amino acid substitutions, additions, and/or deletions are not in the CDRs. In some embodiments, the variant of Lintuzumab has up to about 5 conservative amino acid substitutions. In some embodiments, the variant of Lintuzumab has up to 3 conservative amino acid substitutions.

In some embodiments, the anti-CD74 antibody or antigen-binding fragment thereof provided herein is the antibody designated as cLL1. In some embodiments, the anti-CD74 antibody or antigen-binding fragment thereof provided herein has a VL from cLL1. In some embodiments, the anti-CD74 antibody or antigen-binding fragment thereof provided herein has a VH from cLL1. The anti-CD74 antibody or antigen-binding fragment thereof provided herein can have both a VL and a VH from cLL1. In some embodiments, the anti-CD74 antibody or antigen-binding fragment thereof provided herein has a VL that comprises VL CDRs 1, 2, and 3 from the VL from cLL1. In some embodiments, the anti-CD74 antibody or antigen-binding fragment thereof provided herein has a VH that comprises VH CDRs 1, 2, and 3 from the VH from cLL1. The anti-CD74 antibody or antigen-binding fragment thereof provided herein can have a VL comprising VL CDRs 1, 2, and 3 and a VH comprising VH CDRs 1, 2, and 3 from the VL and VH of cLL1, respectively. In some embodiments, the anti-CD74 antibody or antigen-binding fragment thereof provided herein is a variant of cLL1. The cLL1 variant can have a VL that is a variant of the VL of cLL1 having up to about 5 amino acid substitutions, additions, and/or deletions in SEQ ID NO: 51. The cLL1 variant can have a VH that is a variant of the VH of cLL1 having up to about 5 amino acid substitutions, additions, and/or deletions in SEQ ID NO: 52. The amino acid substitutions, additions, and/or deletions can be in the VH CDRs or VL CDRs. In some embodiments, the amino acid substitutions, additions, and/or deletions are not in the CDRs. In some embodiments, the variant of cLL1 has up to about 5 conservative amino acid substitutions. In some embodiments, the variant of cLL1 has up to 3 conservative amino acid substitutions.

In some embodiments, the anti-CD74 antibody or antigen-binding fragment thereof provided herein is the antibody designated as hLL1. In some embodiments, the anti-CD74 antibody or antigen-binding fragment thereof provided herein has a VL from hLL1. In some embodiments, the anti-CD74 antibody or antigen-binding fragment thereof provided herein has a VH from hLL1. The anti-CD74 antibody or antigen-binding fragment thereof provided herein can have both a VL and a VH from hLL1. In some embodiments, the anti-CD74 antibody or antigen-binding fragment thereof provided herein has a VL that comprises VL CDRs 1, 2, and 3 from the VL from hLL1. In some embodiments, the anti-CD74 antibody or antigen-binding fragment thereof provided herein has a VH that comprises VH CDRs 1, 2, and 3 from the VH from hLL1. The anti-CD74 antibody or antigen-binding fragment thereof provided herein can have a VL comprising VL CDRs 1, 2, and 3 and a VH comprising VH CDRs 1, 2, and 3 from the VL and VH of hLL1, respectively. In some embodiments, the anti-CD74 antibody or antigen-binding fragment thereof provided herein is a variant of hLL1. The hLL1 variant can have a VL that is a variant of the VL of hLL1 having up to about 5 amino acid substitutions, additions, and/or deletions in SEQ ID NO: 53. The hLL1 variant can have a VH that is a variant of the VH of hLL1 having up to about 5 amino acid substitutions, additions, and/or deletions in SEQ ID NO: 54. The amino acid substitutions, additions, and/or deletions can be in the VH CDRs or VL CDRs. In some embodiments, the amino acid substitutions, additions, and/or deletions are not in the CDRs. In some embodiments, the variant of hLL1 has up to about 5 conservative amino acid substitutions. In some embodiments, the variant of hLL1 has up to 3 conservative amino acid substitutions.

In some embodiments, the anti-CD74 antibody or antigen-binding fragment thereof provided herein is the antibody designated as hLL1. In some embodiments, the anti-CD74 antibody or antigen-binding fragment thereof provided herein has a VL from hLL1. In some embodiments, the anti-CD74 antibody or antigen-binding fragment thereof provided herein has a VH from hLL1. The anti-CD74 antibody or antigen-binding fragment thereof provided herein can have both a VL and a VH from hLL1. In some embodiments, the anti-CD74 antibody or antigen-binding fragment thereof provided herein has a VL that comprises VL CDRs 1, 2, and 3 from the VL from hLL1. In some embodiments, the anti-CD74 antibody or antigen-binding fragment thereof provided herein has a VH that comprises VH CDRs 1, 2, and 3 from the VH from hLL1. The anti-CD74 antibody or antigen-binding fragment thereof provided herein can have a VL comprising VL CDRs 1, 2, and 3 and a VH comprising VH CDRs 1, 2, and 3 from the VL and VH of hLL1, respectively. In some embodiments, the anti-CD74 antibody or antigen-binding fragment thereof provided herein is a variant of hLL1. The hLL1 variant can have a VL that is a variant of the VL of hLL1 having up to about 5 amino acid substitutions, additions, and/or deletions in SEQ ID NO: 53. The hLL1 variant can have a VH that is a variant of the VH of hLL1 having up to about 5 amino acid substitutions, additions, and/or deletions in SEQ ID NO: 54. The amino acid substitutions, additions, and/or deletions can be in the VH CDRs or VL CDRs. In some embodiments, the amino acid substitutions, additions, and/or deletions are not in the CDRs. In some embodiments, the variant of hLL1 has up to about 5 conservative amino acid substitutions. In some embodiments, the variant of hLL1 has up to 3 conservative amino acid substitutions.

In some embodiments, the anti-Aβ antibody or antigen-binding fragment thereof provided herein is the antibody designated as Aducanumab. In some embodiments, the anti-Aβ antibody or antigen-binding fragment thereof provided herein has a VL from Aducanumab. In some embodiments, the anti-Aβ antibody or antigen-binding fragment thereof provided herein has a VH from Aducanumab. The anti-Aβ antibody or antigen-binding fragment thereof provided herein can have both a VL and a VH from Aducanumab. In some embodiments, the anti-Aβ antibody or antigen-binding fragment thereof provided herein has a VL that comprises VL CDRs 1, 2, and 3 from the VL from Aducanumab. In some embodiments, the anti-Aβ antibody or antigen-binding fragment thereof provided herein has a VH that comprises VH CDRs 1, 2, and 3 from the VH from Aducanumab. The anti-Aβ antibody or antigen-binding fragment thereof provided herein can have a VL comprising VL CDRs 1, 2, and 3 and a VH comprising VH CDRs 1, 2, and 3 from the VL and VH of Aducanumab, respectively. In some embodiments, the anti-Aβ antibody or antigen-binding fragment thereof provided herein is a variant of Aducanumab. The Aducanumab variant can have a VL that is a variant of the VL of Aducanumab having up to about 5 amino acid substitutions, additions, and/or deletions in SEQ ID NO: 61. The Aducanumab variant can have a VH that is a variant of the VH of Aducanumab having up to about 5 amino acid substitutions, additions, and/or deletions in SEQ ID NO: 62 or SEQ ID NO. 63. The amino acid substitutions, additions, and/or deletions can be in the VH CDRs or VL CDRs. In some embodiments, the amino acid substitutions, additions, and/or deletions are not in the CDRs. In some embodiments, the variant of Aducanumab has up to about 5 conservative amino acid substitutions. In some embodiments, the variant of Aducanumab has up to 3 conservative amino acid substitutions.

In some embodiments, the anti-Aβ antibody or antigen-binding fragment thereof provided herein is the antibody designated as BAN2401. In some embodiments, the anti-Aβ antibody or antigen-binding fragment thereof provided herein has a VL from BAN2401. In some embodiments, the anti-Aβ antibody or antigen-binding fragment thereof provided herein has a VH from BAN2401. The anti-Aβ antibody or antigen-binding fragment thereof provided herein can have both a VL and a VH from BAN2401. In some embodiments, the anti-Aβ antibody or antigen-binding fragment thereof provided herein has a VL that comprises VL CDRs 1, 2, and 3 from the VL from BAN2401. In some embodiments, the anti-Aβ antibody or antigen-binding fragment thereof provided herein has a VH that comprises VH CDRs 1, 2, and 3 from the VH from BAN2401. The anti-Aβ antibody or antigen-binding fragment thereof provided herein can have a VL comprising VL CDRs 1, 2, and 3 and a VH comprising VH CDRs 1, 2, and 3 from the VL and VH of BAN2401, respectively. In some embodiments, the anti-Aβ antibody or antigen-binding fragment thereof provided herein is a variant of BAN2401. The BAN2401 variant can have a VL that is a variant of the VL of BAN2401 having up to about 5 amino acid substitutions, additions, and/or deletions in SEQ ID NO: 70. The BAN2401 variant can have a VH that is a variant of the VH of BAN2401 having up to about 5 amino acid substitutions, additions, and/or deletions in SEQ ID NO: 71. The amino acid substitutions, additions, and/or deletions can be in the VH CDRs or VL CDRs. In some embodiments, the amino acid substitutions, additions, and/or deletions are not in the CDRs. In some embodiments, the variant of BAN2401 has up to about 5 conservative amino acid substitutions. In some embodiments, the variant of BAN2401 has up to 3 conservative amino acid substitutions.

In some embodiments, the anti-Aβ antibody or antigen-binding fragment thereof provided herein is the antibody designated as Gantenerumab. In some embodiments, the anti-Aβ antibody or antigen-binding fragment thereof provided herein has a VL from Gantenerumab. In some embodiments, the anti-Aβ antibody or antigen-binding fragment thereof provided herein has a VH from Gantenerumab. The anti-Aβ antibody or antigen-binding fragment thereof provided herein can have both a VL and a VH from Gantenerumab. In some embodiments, the anti-Aβ antibody or antigen-binding fragment thereof provided herein has a VL that comprises VL CDRs 1, 2, and 3 from the VL from Gantenerumab. In some embodiments, the anti-Aβ antibody or antigen-binding fragment thereof provided herein has a VH that comprises VH CDRs 1, 2, and 3 from the VH from Gantenerumab. The anti-Aβ antibody or antigen-binding fragment thereof provided herein can have a VL comprising VL CDRs 1, 2, and 3 and a VH comprising VH CDRs 1, 2, and 3 from the VL and VH of Gantencrumab, respectively. In some embodiments, the anti-Aβ antibody or antigen-binding fragment thereof provided herein is a variant of Gantenerumab. The Gantenerumab variant can have a VL that is a variant of the VL of Gantenerumab having up to about 5 amino acid substitutions, additions, and/or deletions in SEQ ID NO: 78. The Gantenerumab variant can have a VH that is a variant of the VH of Gantenerumab having up to about 5 amino acid substitutions, additions, and/or deletions in SEQ ID NO: 79. The amino acid substitutions, additions, and/or deletions can be in the VH CDRs or VL CDRs. In some embodiments, the amino acid substitutions, additions, and/or deletions are not in the CDRs. In some embodiments, the variant of Gantenerumab has up to about 5 conservative amino acid substitutions. In some embodiments, the variant of Gantenerumab has up to 3 conservative amino acid substitutions.

In some embodiments, the anti-Aβ antibody or antigen-binding fragment thereof provided herein is the antibody designated as Crenczumab. In some embodiments, the anti-Aβ antibody or antigen-binding fragment thereof provided herein has a VL from Crenczumab. In some embodiments, the anti-Aβ antibody or antigen-binding fragment thereof provided herein has a VH from Crenczumab. The anti-Aβ antibody or antigen-binding fragment thereof provided herein can have both a VL and a VH from Crenczumab. In some embodiments, the anti-Aβ antibody or antigen-binding fragment thereof provided herein has a VL that comprises VL CDRs 1, 2, and 3 from the VL from Crenczumab. In some embodiments, the anti-Aβ antibody or antigen-binding fragment thereof provided herein has a VH that comprises VH CDRs 1, 2, and 3 from the VH from Crenczumab. The anti-Aβ antibody or antigen-binding fragment thereof provided herein can have a VL comprising VL CDRs 1, 2, and 3 and a VH comprising VH CDRs 1, 2, and 3 from the VL and VH of Crenczumab, respectively. In some embodiments, the anti-Aβ antibody or antigen-binding fragment thereof provided herein is a variant of Crenezumab. The Crenczumab variant can have a VL that is a variant of the VL of Crenezumab having up to about 5 amino acid substitutions, additions, and/or deletions in SEQ ID NO: 86. The Crenezumab variant can have a VH that is a variant of the VH of Crenezumab having up to about 5 amino acid substitutions, additions, and/or deletions in SEQ ID NO: 87. The amino acid substitutions, additions, and/or deletions can be in the VH CDRs or VL CDRs. In some embodiments, the amino acid substitutions, additions, and/or deletions are not in the CDRs. In some embodiments, the variant of Crenezumab has up to about 5 conservative amino acid substitutions. In some embodiments, the variant of Crenczumab has up to 3 conservative amino acid substitutions.

In some embodiments, the anti-Aβ antibody or antigen-binding fragment thereof provided herein is the antibody designated as AD38. In some embodiments, the anti-Aβ antibody or antigen-binding fragment thereof provided herein has a VL from AD38. In some embodiments, the anti-Aβantibody or antigen-binding fragment thereof provided herein has a VH from AD38. The anti-Aβantibody or antigen-binding fragment thereof provided herein can have both a VL and a VH from AD38. In some embodiments, the anti-Aβ antibody or antigen-binding fragment thereof provided herein has a VL that comprises VL CDRs 1, 2, and 3 from the VL from AD38. In some embodiments, the anti-Aβ antibody or antigen-binding fragment thereof provided herein has a VH that comprises VH CDRs 1, 2, and 3 from the VH from AD38. The anti-Aβ antibody or antigen-binding fragment thereof provided herein can have a VL comprising VL CDRs 1, 2, and 3 and a VH comprising VH CDRs 1, 2, and 3 from the VL and VH of AD38, respectively. In some embodiments, the anti-Aβ antibody or antigen-binding fragment thereof provided herein is a variant of AD38. The AD38 variant can have a VL that is a variant of the VL of AD38 having up to about 5 amino acid substitutions, additions, and/or deletions in SEQ ID NO: 86. The AD38 variant can have a VH that is a variant of the VH of AD38 having up to about 5 amino acid substitutions, additions, and/or deletions in SEQ ID NO: 87. The amino acid substitutions, additions, and/or deletions can be in the VH CDRs or VL CDRs. In some embodiments, the amino acid substitutions, additions, and/or deletions are not in the CDRs. In some embodiments, the variant of AD38 has up to about 5 conservative amino acid substitutions. In some embodiments, the variant of AD38 has up to 3 conservative amino acid substitutions.

In some embodiments, the anti-CD22 antibody or antigen-binding fragment thereof provided herein is the antibody designated as SM06. In some embodiments, the anti-CD22 antibody or antigen-binding fragment thereof provided herein has a VL from SM06. In some embodiments, the anti-CD22 antibody or antigen-binding fragment thereof provided herein has a VH from SM06. The anti-CD22 antibody or antigen-binding fragment thereof provided herein can have both a VL and a VH from SM06. In some embodiments, the anti-CD22 antibody or antigen-binding fragment thereof provided herein has a VL that comprises VL CDRs 1, 2, and 3 from the VL from SM06. In some embodiments, the anti-CD22 antibody or antigen-binding fragment thereof provided herein has a VH that comprises VH CDRs 1, 2, and 3 from the VH from SM06. The anti-CD22 antibody or antigen-binding fragment thereof provided herein can have a VL comprising VL CDRs 1, 2, and 3 and a VH comprising VH CDRs 1, 2, and 3 from the VL and VH of SM06, respectively. In some embodiments, the anti-CD22 antibody or antigen-binding fragment thereof provided herein is a variant of SM06. The SM06 variant can have a VL that is a variant of the VL of SM06 having up to about 5 amino acid substitutions, additions, and/or deletions in SEQ ID NO: 9. The SM06 variant can have a VH that is a variant of the VH of SM06 having up to about 5 amino acid substitutions, additions, and/or deletions in SEQ ID NO: 10. The amino acid substitutions, additions, and/or deletions can be in the VH CDRs or VL CDRs. In some embodiments, the amino acid substitutions, additions, and/or deletions are not in the CDRs. In some embodiments, the variant of SM06 has up to about 5 conservative amino acid substitutions. In some embodiments, the variant of SM06 has up to 3 conservative amino acid substitutions.

In some embodiments, the anti-CD74 antibody or antigen-binding fragment thereof provided herein is the antibody designated as hLL1. In some embodiments, the anti-CD74 antibody or antigen-binding fragment thereof provided herein has a VL from hLL1. In some embodiments, the anti-CD74 antibody or antigen-binding fragment thereof provided herein has a VH from hLL1. The anti-CD74 antibody or antigen-binding fragment thereof provided herein can have both a VL and a VH from hLL1. In some embodiments, the anti-CD74 antibody or antigen-binding fragment thereof provided herein has a VL that comprises VL CDRs 1, 2, and 3 from the VL from hLL1. In some embodiments, the anti-CD74 antibody or antigen-binding fragment thereof provided herein has a VH that comprises VH CDRs 1, 2, and 3 from the VH from hLL1. The anti-CD74 antibody or antigen-binding fragment thereof provided herein can have a VL comprising VL CDRs 1, 2, and 3 and a VH comprising VH CDRs 1, 2, and 3 from the VL and VH of hLL1, respectively. In some embodiments, the anti-CD74 antibody or antigen-binding fragment thereof provided herein is a variant of hLL1. The hLL1 variant can have a VL that is a variant of the VL of hLL1 having up to about 5 amino acid substitutions, additions, and/or deletions in SEQ ID NO: 53. The hLL1 variant can have a VH that is a variant of the VH of hLL1 having up to about 5 amino acid substitutions, additions, and/or deletions in SEQ ID NO: 54. The amino acid substitutions, additions, and/or deletions can be in the VH CDRs or VL CDRs. In some embodiments, the amino acid substitutions, additions, and/or deletions are not in the CDRs. In some embodiments, the variant of hLL1 has up to about 5 conservative amino acid substitutions. In some embodiments, the variant of hLL1 has up to 3 conservative amino acid substitutions.

In some embodiments, the anti-CD22 antibodies or antigen-binding fragments that can be used in methods disclosed herein comprise a VH or VL that has at least one framework (FR) region. The FR regions for VL can be from the Vκ10 murine germline family. In some embodiments, the FR1, FR2, FR3, and FR4 for VL can have the amino acid sequence of SEQ ID NOs: 30, 31, 32, and 33, respectively (the SM03 VL framework sequences). In some embodiments, the FR region for VH can be from the VH5 murine germline family. In some embodiments, the FR1, FR2, FR3, and FR4 for VH can have the amino acid sequence of SEQ ID NOs: 34, 35, 36, and 37, respectively (the SM03 VH framework sequences). In some embodiments, the FRI region for VL can be from the VκID human germline family, the FR2 region for VL can be from the VκI human germline family, the FR3 region for VL can be from the Vκ1 human germline family, and the FR4 region for VL can be from the VκJ1 human germline family. In some embodiments, the FR1, FR2, FR3, and FR4 for VL can have the amino acid sequences of SEQ ID NOs: 22, 23, 24, and 25, respectively (the SM06 VL framework sequences). In some embodiments, the FRI region for VH can be from the V_{H}3 human germline family; the FR2 region regions for VH can be from the V_{H}3 human germline family; the FR3 region for VH can be from the V_{H}3 human germline family; and the FR4 region regions for VH can be from the V_{H}J5 human germline family. In some embodiments, the FR1, FR2, FR3, and FR4 for VH can have the amino acid sequences of SEQ ID NOs: 26, 27, 28, and 29, respectively (the SM06 VH framework sequences).

The present disclosure further contemplates additional variants and equivalents that are substantially homologous to the recombinant, monoclonal, chimeric, humanized, and human antibodies, or antibody fragments thereof, described herein. In some embodiments, it is desirable to improve the binding affinity of the antibody. In some embodiments, it is desirable to modulate biological properties of the antibody, including but not limited to, specificity, thermostability, expression level, effector function(s), glycosylation, immunogenicity, and/or solubility. Those skilled in the art will appreciate that amino acid changes may alter post-translational processes of an antibody, such as changing the number or position of glycosylation sites or altering membrane anchoring characteristics.

Variations can be a substitution, deletion, or insertion of one or more nucleotides encoding the antibody or polypeptide that results in a change in the amino acid sequence as compared with the native antibody or polypeptide sequence. In some embodiments, amino acid substitutions are the result of replacing one amino acid with another amino acid having similar structural and/or chemical properties, such as the replacement of a leucine with a serine, e.g., conservative amino acid replacements. Insertions or deletions can be in the range of about 1 to 5 amino acids. In some embodiments, the substitution, deletion, or insertion includes less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the parent molecule. In some embodiments, variations in the amino acid sequence that are biologically useful and/WO or relevant can be determined by systematically making insertions, deletions, or substitutions in the sequence and testing the resulting variant proteins for activity as compared to the parent protein.

In some embodiments, an anti-CD22, anti-CD33, or anti-CD74 bispecific antibody or antigen-binding fragment thereof that can be used in methods provided herein is a chimeric antibody or antigen-binding fragment. In some embodiments, an anti-CD22, anti-CD33, or anti-CD74 bispecific antibody or antigen-binding fragment thereof that can be used in methods provided herein is a humanized antibody or antigen-binding fragment. In some embodiments, an anti-CD22, anti-CD33 or anti-CD74 antibody or antigen-binding fragment thereof comprises a VL CDR1, VL CDR2, VL CDR3, VH CDR1, VH CDR2, and/or VH CDR3 from an antibody or antigen-binding fragment with the respective antigen specificities as described herein. In some embodiments, an anti-CD22, anti-CD33, or anti-CD74 bispecific antibody or antigen-binding fragment thereof comprises a variant of a respective anti-CD22, anti-CD33, or anti-CD74 bispecific antibody or antigen-binding fragment described herein. In some embodiments, a variant of an anti-CD22, anti-CD33 or anti-CD74 antibody or antigen-binding fragment comprises one to 30 amino acid substitutions, additions, and/or deletions in the respective anti-CD22, anti-CD33 or anti-CD74 antibody or antigen-binding fragment. In some embodiments, a variant of an anti-CD22, anti-CD33 or anti-CD74 bispecific antibody or antigen-binding fragment comprises one to 25 amino acid substitutions, additions, and/or deletions in the respective anti-CD22, anti-CD33 or anti-CD74 antibody or antigen-binding fragment. In some embodiments, a variant of an anti-CD22, anti-CD33 or anti-CD74 bispecific antibody or antigen-binding fragment comprises one to 20 substitutions, additions, and/or deletions in the respective anti-CD22, anti-CD33 or anti-CD74 antibody or antigen-binding fragment. In some embodiments, a variant of an anti-CD22, anti-CD33 or anti-CD74 bispecific antibody or antigen-binding fragment comprises one to 15 substitutions, additions, and/or deletions in the respective anti-CD22, anti-CD33 or anti-CD74 antibody or antigen-binding fragment. In some embodiments, a variant of an anti-CD22, anti-CD33 or anti-CD74 bispecific antibody or antigen-binding fragment comprises one to 10 substitutions, additions, and/or deletions in the respective anti-CD22, anti-CD33 or anti-CD74 antibody or antigen-binding fragment. In some embodiments, a variant of an anti-CD22, anti-CD33 or anti-CD74 bispecific antibody or antigen-binding fragment comprises one to five conservative amino acid substitutions, additions, and/or deletions in the respective anti-CD22, anti-CD33 or anti-CD74 antibody or antigen-binding fragment. In some embodiments, a variant of an anti-CD22, anti-CD33 or anti-CD74 bispecific antibody or antigen-binding fragment comprises one to three amino acid substitutions, additions, and/or deletions in the respective anti-CD22, anti-CD33 or anti-CD74 antibody or antigen-binding fragment. In some embodiments, the amino acid substitutions, additions, and/or deletions are conservative amino acid substitutions. In some embodiments, the conservative amino acid substitution(s) is in a CDR of the antibody or antigen-binding fragment. In some embodiments, the conservative amino acid substitution(s) is not in a CDR of the antibody or antigen-binding fragment. In some embodiments, the conservative amino acid substitution(s) is in a framework region of the antibody or antigen-binding fragment.

It is known in the art that the constant region(s) of an antibody mediates several effector functions, and these effector functions can vary depending on the isotype of the antibody. For example, binding of the Cl component of complement to the Fc region of IgG or IgM antibodies (bound to antigen) activates the complement system. Activation of complement is important in the opsonization and lysis of cell pathogens. The activation of complement also stimulates the inflammatory response and can be involved in autoimmune hypersensitivity. In addition, the Fc region of an antibody can bind a cell expressing a Fc receptor (FcR). There are a number of Fc receptors which are specific for different classes of antibody, including IgG (gamma receptors), IgE (epsilon receptors), IgA (alpha receptors) and IgM (mu receptors). Binding of antibody to Fc receptors on cell surfaces triggers many important and diverse biological responses including engulfment and destruction of antibody-coated particles, clearance of immune complexes, lysis of antibody-coated target cells by killer cells (called antibody-dependent cell cytotoxicity or ADCC), release of inflammatory mediators, placental transfer, and control of immunoglobulin production. In some embodiments, anti-CD22, anti-CD33 or anti-CD74 bispecific antibody or antigen-binding fragment described herein comprise at least one constant region of a human IgA antibody. In some embodiments, anti-CD22, anti-CD33 or anti-CD74 bispecific antibody or antigen-binding fragment described herein comprise at least one constant region of a human IgD antibody. In some embodiments, anti-CD22, anti-CD33 or anti-CD74 bispecific antibody or antigen-binding fragment described herein comprise at least one constant region of a human IgE antibody. In some embodiments, anti-CD22, anti-CD33 or anti-CD74 bispecific antibody or antigen-binding fragment described herein comprise at least one constant region of a human IgG antibody. In some embodiments, anti-CD22, anti-CD33 or anti-CD74 bispecific antibody or antigen-binding fragment described herein comprise at least one constant region of a human IgM antibody. In some embodiments, anti-CD22, anti-CD33 or anti-CD74 bispecific antibody or antigen-binding fragment described herein comprise at least one constant region of a human IgG1 antibody. In some embodiments, anti-CD22, anti-CD33 or anti-CD74 bispecific antibody or antigen-binding fragment described herein comprise at least one constant region of a human IgG2 antibody. In some embodiments, anti-CD22, anti-CD33 or anti-CD74 bispecific antibody or antigen-binding fragment described herein comprise at least one constant region of a human IgG3 antibody. In some embodiments, anti-CD22, anti-CD33 or anti-CD74 bispecific antibody or antigen-binding fragment described herein comprise at least one constant region of a human IgG4 antibody.

In some embodiments, at least one or more of the constant regions has been modified or deleted in the anti-CD22, anti-CD33 or anti-CD74 bispecific antibody or antigen-binding fragment described herein. In some embodiments, the antibodies comprise modifications to one or more of the three heavy chain constant regions (CH1, CH2 or CH3) and/or to the light chain constant region (CL). In some embodiments, the heavy chain constant region of the modified antibodies comprises at least one human constant region. In some embodiments, the heavy chain constant region of the modified antibodies comprises more than one human constant region. In some embodiments, modifications to the constant region comprise additions, deletions, or substitutions of one or more amino acids in one or more regions. In some embodiments, one or more regions are partially or entirely deleted from the constant regions of the modified antibodies. In some embodiments, the entire CH2 domain has been removed from an antibody (ΔCH2 constructs). In some embodiments, a deleted constant region is replaced by a short amino acid spacer that provides some of the molecular flexibility typically imparted by the absent constant region. In some embodiments, a modified antibody comprises a CH3 domain directly fused to the hinge region of the antibody. In some embodiments, a modified antibody comprises a peptide spacer inserted between the hinge region and modified CH2 and/or CH3 domains.

In some embodiments, an anti-CD22, anti-CD33 or anti-CD74 bispecific antibody or antigen-binding fragment comprises a Fc region. In some embodiments, the Fc region is fused via a hinge. The hinge can be an IgG1 hinge, an IgG2 hinge, or an IgG3 hinge. The amino acid sequences of the Fc region of human IgG1, IgG2, IgG3, and IgG4 are known to those of ordinary skill in the art. In some cases, Fc regions with amino acid variations have been identified in native antibodies. In some embodiments, the modified antibodies (e.g., modified Fc region) provide for altered effector functions that, in turn, affect the biological profile of the antibody. For example, in some embodiments, the Fc regions of antibodies or antigen-binding fragments provided herein are modified to enhance their ability to cross the blood-brain barrier (BBB). In some embodiments, the deletion or inactivation (through point mutations or other means) of a constant region reduces Fc receptor binding of the modified antibody as it circulates. In some embodiments, the constant region modifications reduce the immunogenicity of the antibody. In some embodiments, the constant region modifications increase the serum half-life of the antibody. In some embodiments, the constant region modifications reduce the serum half-life of the antibody. In some embodiments, the constant region modifications decrease or remove ADCC and/or complement dependent cytotoxicity (CDC) of the antibody. In some embodiments, specific amino acid substitutions in a human IgG1 Fc region with corresponding IgG2 or IgG4 residues reduce effector functions (e.g., ADCC and CDC) in the modified antibody. In some embodiments, an antibody does not have one or more effector functions (e.g., “effectorless” antibodies). In some embodiments, the antibody has no ADCC activity and/or no CDC activity. In some embodiments, the antibody does not bind an Fc receptor and/or complement factors. In some embodiments, the antibody has no effector function(s). In some embodiments, the constant region modifications increase or enhance ADCC and/or CDC of the antibody. In some embodiments, the constant region is modified to eliminate disulfide linkages or oligosaccharide moieties. In some embodiments, the constant region is modified to add/substitute one or more amino acids to provide one or more cytotoxin, oligosaccharide, or carbohydrate attachment sites. In some embodiments, an anti-CD22, anti-CD33 or anti-CD74 bispecific antibody or antigen-binding fragment comprises a variant Fc region that is engineered with substitutions at specific amino acid positions as compared to a native Fc region. In some embodiments, an anti-CD22, anti-CD33 or anti-CD74 bispecific antibody or antigen-binding fragment described herein comprises an IgG1 heavy chain constant region that comprises one or more amino acid substitutions selected from the group consisting of K214R, L234A, L235E, G237A, D356E, and L358M, per EU numbering. In some embodiments, the IgG1 heavy chain constant region comprises one or more amino acid substitutions selected from the group consisting of K214R, L234A, L235E, G237A, A330S, P331S, D356E, and L358M, per EU numbering. In some embodiments, the IgG1 heavy chain constant region comprises one or more amino acid substitutions selected from the group consisting of K214R, C226S, C229S, and P238S, per EU numbering. In some embodiments, the IgG1 heavy chain constant region comprises one or more amino acid substitutions selected from the group consisting of K214R, D356E, and L358M, per EU numbering. In some embodiments, the IgG1 heavy chain constant region comprises one or more amino acid substitutions selected from the group consisting of S131C, K133R, G137E, G138S, Q196K, 1199T, N203D, K214R, C226S, C229S, and P238S, per EU numbering.

In some embodiments, variants can include addition of amino acid residues at the amino- and/or carboxyl-terminal end of the antibody or polypeptide. The length of additional amino acids residues can range from one residue to a hundred or more residues. In some embodiments, a variant comprises an N-terminal methionyl residue. In some embodiments, the variant comprises an additional polypeptide/protein (e.g., Fc region) to create a fusion protein. In some embodiments, a variant is engineered to be detectable and may comprise a detectable label and/or protein (e.g., a fluorescent tag or an enzyme).

Therapeutic antibodies can be re-engineered to facilitate transport across the BBB via various mechanism, including, for example, receptor mediated transcytosis, adsorptive transcytosis, carrier mediated transport, paracellular transport and diffusion. Among all, receptor mediated transcytosis (R.M. T.) has been widely explored to facilitate the entry of antibody. The use of R. M. T. includes transferrin receptor, insulin receptor, low density lipoprotein receptor (LDL receptor), CD98, TMEM50A, and other surface receptors on endothelial cells that can perform transcytosis upon binding. Antibody binding to the transferrin, insulin receptor, CD98 and TEME50A can elicit transcytosis of the whole complex. In case of LDL receptor, binding of apolipoprotein can trigger the transcytosis process. Therefore, therapeutic antibody linked to an antibody fragment or apolipoprotein is proposed and validated to enhance BBB crossing in animal model.

As such, the anti-CD22, anti-CD33 or anti-CD74 bispecific antibodies or antigen-binding fragments provided herein can be engineered to enhance its ability to cross BBB. In some embodiments, the anti-CD22, anti-CD33 or anti-CD74 bispecific antibodies or antigen-binding fragments provided herein are fused to a second antibody or antigen-binding fragment that binds to the transferrin, insulin receptor, CD98 or TEME50A. In some embodiments, the anti-CD22, anti-CD33 or anti-CD74 bispecific antibodies or antigen-binding fragments provided herein can be fused to a second antibody or antigen-binding fragment that binds to transferrin. In some embodiments, for example, the anti-CD22, anti-CD33 or anti-CD74 bispecific antibodies or antigen-binding fragment can be fused to a transferrin receptor binding Fab fragment. In some embodiments, the anti-CD22, anti-CD33 or anti-CD74 bispecific antibodies or antigen-binding fragments provided herein can be fused to a second antibody or antigen-binding fragment that binds to insulin receptor. In some embodiments, the anti-CD22, anti-CD33 or anti-CD74 bispecific antibodies or antigen-binding fragments provided herein can be fused to a second antibody or antigen-binding fragment that binds to CD98. In some embodiments, the anti-CD22, anti-CD33 or anti-CD74 bispecific antibodies or antigen-binding fragments provided herein can be fused to a second antibody or antigen-binding fragment that binds to TEME50A. In some embodiments, the anti-CD22, anti-CD33 or anti-CD74 bispecific antibodies or antigen-binding fragments provided herein are fused to an apolipoprotein.

The variant antibodies or antigen-binding fragments described herein can be generated using methods known in the art, including but not limited to, site-directed mutagenesis, alanine scanning mutagenesis, and PCR mutagenesis.

In some embodiments, a variant of an anti-CD22, anti-CD33 or anti-CD74 bispecific antibody or antigen-binding fragment disclosed herein can retain the ability to bind to their corresponding antigen targets to a similar extent, the same extent, or to a higher extent, as the parent antibody or antigen-binding fragment. In some embodiments, the variant can be at least about 80%, about 85%, about 90%, about 91%, about 92%, about 93%, about 94%, about 95%, about 96%, about 97%, about 98%, about 99% or more identical in amino acid sequence to the parent antibody or antigen-binding fragment. In certain embodiments, a variant of an anti-CD22, anti-CD33 or anti-CD74 bispecific antibody or antigen-binding fragment comprises the amino acid sequence of the respective parent anti-CD22, anti-CD33 or anti-CD74 antibody or antigen-binding fragment with one or more conservative amino acid substitution. Conservative amino acid substitutions are known in the art and include amino acid substitutions in which one amino acid having certain physical and/or chemical properties is exchanged for another amino acid that has the same or similar chemical or physical properties.

In some embodiments, a variant of an anti-CD22, anti-CD33 or anti-CD74 bispecific antibody or antigen-binding fragment comprises the amino acid sequence of the parent antibody or antigen-binding fragment with one or more non-conservative amino acid substitutions. In some embodiments, a variant of an anti-CD22, anti-CD33 or anti-CD74 bispecific antibody or antigen-binding fragment comprises the amino acid sequence of the parent binding antibody or antigen-binding fragment with one or more non-conservative amino acid substitution, wherein the one or more non-conservative amino acid substitutions do not interfere with or inhibit one or more biological activities of the variant (e.g., CD22 binding for variants of anti-CD22 bispecific antibody or antigen-binding fragment, CD33 binding for variants of anti-CD33 bispecific antibody or antigen-binding fragment, or CD74 binding for variants of anti-CD74 bispecific antibody or antigen-binding fragment). In certain embodiments, the one or more conservative amino acid substitutions and/or the one or more non-conservative amino acid substitutions can enhance a biological activity of the variant, such that the biological activity of the functional variant is increased as compared to the parent binding moiety.

In some embodiments, anti-CD22, anti-CD33 or anti-CD74 bispecific antibodies or antigen-binding fragments described herein are chemically modified naturally or by intervention. In some embodiments, the anti-CD22, anti-CD33 or anti-CD74 bispecific antibodies or antigen-binding fragments have been chemically modified by glycosylation, acetylation, pegylation, phosphorylation, amidation, derivatization by known protecting/blocking groups, proteolytic cleavage, and/or linkage to a cellular ligand or other protein. Any of numerous chemical modifications can be carried out by known techniques. The anti-CD22, anti-CD33 or anti-CD74 bispecific antibodies or antigen-binding fragments can comprise one or more analogs of an amino acid (including, for example, unnatural amino acids), as well as other modifications known in the art.

The anti-CD22, anti-CD33 or anti-CD74 bispecific antibodies or antigen-binding fragments of the present disclosure can be analyzed for their physical, chemical and/or biological properties by various methods known in the art. In some embodiments, an anti-CD22, anti-CD33 or anti-CD74 bispecific antibody is tested for its ability to bind to their corresponding antigens, such as CD22 (e.g., human CD22), CD33 or CD74, respectively. Binding assays include, but are not limited to, surface plasmon resonance (e.g., Biacore), ELISA, and FACS. In some embodiments, the dissociation constant of the binding agent (e.g., an antibody) for their corresponding antigens is the dissociation constant determined by surface plasmon resonance (e.g., BIAcore). In addition, antibodies can be evaluated for solubility, stability, thermostability, viscosity, expression levels, expression quality, and/or purification efficiency.

All of the anti-CD22, anti-CD33 or anti-CD74 bispecific antibodies or antigen-binding fragments described herein bind to their corresponding antigen with a dissociation constant (K_{D}) within the range of 0.02 to 10 nM. In some embodiments, an anti-CD22, anti-CD33 or anti-CD74 bispecific antibody or antigen-binding fragment used in the methods disclosed herein binds to their corresponding antigens with a dissociation constant (K_{D}) of about 100 nM or less, about 40 nM or less, about 20 nM or less, about 10 nM or less, about 1 nM or less, about 0.1 nM or less, 50 pM or less, 10 pM or less, or 1 pM or less. In some embodiments, the K_{D} is about 20 nM or less. In some embodiments, the K_{D} is about 10 nM or less. In some embodiments, the K_{D} is about of about 5 nM or less. In some embodiments, the K_{D} is about 2 nM or less. In some embodiments, the K_{D} is about 1.5 nM or less. In some embodiments, the K_{D} is about 1 nM or less. In some embodiments, the K_{D} is about 0.5 nM or less. In some embodiments, the K_{D} is about 0.1 nM or less. In some embodiments, the K_{D} is about 50 pM or less. In some embodiments, the K_{D} is about 10 pM or less.

In some embodiments, an anti-CD22, anti-CD33 or anti-CD74 bispecific antibody or antigen-binding fragment binds to their corresponding antigens with a K_{D} within the range of 0.1-1 nM, 0.5-5 nM, 1-10 nM, 1-5 nM, 5-50 nM, 10-100 nM, or 50-500 nM. In some embodiments, the K_{D} is within the range of 0.1-1 nM. In some embodiments, the K_{D} is within the range of 0.5-5 nM. In some embodiments, the K_{D} is within the range of 1-10 nM. In some embodiments, the K_{D} is within the range of 1-5 nM. In some embodiments, the K_{D} is within the range of 5-50 nM. In some embodiments, the K_{D} is within the range of 10-100 nM. In some embodiments, the K_{D} is within the range of 50-500 nM.

In some embodiments, an anti-CD22, anti-CD33 or anti-CD74 bispecific antibody or antigen-binding fragment used in the methods disclosed herein binds to their corresponding antigens with an association constant (K_{A}) of about 0.8×10⁹ M⁻¹. In some embodiments, an anti-CD22, anti-CD33 or anti-CD74 bispecific antibody or antigen-binding fragment used in the methods disclosed herein binds to their corresponding antigens with a K_{A} of about 1×10⁶ M⁻¹ or more, about 1×10⁷ M⁻¹ or more, about 1×10⁸ M⁻¹ or more, about 5×10⁸ M⁻¹ or more, about 8×10⁸ M⁻¹ or more, about 1×10⁹ M⁻¹ or more, about 5×10⁹ M⁻¹ or more, about 1×10¹⁰ M⁻¹ or more, about 5×10¹⁰ M⁻¹ or more, about 1×10¹¹ M⁻¹ or more, about 5×10¹¹ M⁻¹ or more, or about 1×10¹² M⁻¹ or more. In some embodiments, the K_{A} is about 1×10⁷ M⁻¹ or more. In some embodiments, the K_{A} is about 5×10⁷ M⁻¹ or more. In some embodiments, the K_{A} is about 1×10⁸ M⁻¹ or more. In some embodiments, the K_{A} is about 5×10¹⁰ M⁻¹ or more. In some embodiments, the K_{A} is about 8×10⁸ M⁻¹ or more. In some embodiments, the K_{A} is about 1×10⁹ M⁻¹ or more. In some embodiments, the K_{A} is about 5×10⁹ M⁻¹ or more. In some embodiments, the K_{A} is about 1×10¹⁰ M⁻¹ or more.

In some embodiments, an anti-CD22, anti-CD33 or anti-CD74 bispecific antibody or antigen-binding fragment used in the methods disclosed herein binds to their corresponding antigens with a K_{A} within the range of about 1×10⁶-1×10⁷ M⁻¹, 5×10⁶-5×10⁷ M⁻¹, 1×10⁷-1×10⁸ M⁻¹, 5×10⁷-5×10⁸ M⁻¹, 1×10⁸-5×10⁸ M⁻¹, 1×10⁸-1×10⁹ M⁻¹, 5×10⁸-1×10⁹ M⁻¹, 5×10⁸-5×10⁹ M⁻¹, 1×10⁹-1×10¹⁰ M⁻¹, 5×10⁹-5×10¹⁰ M⁻¹, 1×10¹⁰-1×10¹¹ M⁻¹, 5×10¹⁰-5×10¹¹ M⁻¹, 1×10¹¹-1×10¹² M⁻¹, or 5×10¹¹-5×10¹² M⁻¹. In some embodiments, the K_{A} is within the range of about 1×10⁶-1×10⁷ M⁻¹. In some embodiments, the K_{A} is within the range of about 1×10⁷-1×10⁸ M⁻¹. In some embodiments, the K_{A} is within the range of about 1×10⁸-1×10⁹ M⁻¹. In some embodiments, the K_{A} is within the range of about 5×10⁸-1×10⁹ M⁻¹. In some embodiments, the K_{A} is within the range of about 5×10⁸-5×10⁹ M⁻¹. In some embodiments, the K_{A} is within the range of about 1×10⁹-1×10¹⁰ M⁻¹.

In some embodiments, an anti-CD22, anti-CD33 or anti-CD74 bispecific antibody or antigen-binding fragment used in the methods disclosed herein dissociates from their corresponding human antigens with a kd of 0.0685 RU s⁻¹ or less, or 0.0137 RU s⁻¹ or less, as determined by SPR (e.g., BIAcore). In some embodiments, an anti-CD22, anti-CD33 or anti-CD74 bispecific antibody or antigen-binding fragment used in the methods disclosed herein dissociates from their corresponding human antigens with a kd of about 0.5 RU s⁻¹ or less, about 0.2 RU s⁻¹ or less, about 0.1 RU s⁻¹ or less, about 0.08 RU s⁻¹ or less, about 0.06 RU s⁻¹ or less, about 0.05 RU s⁻¹ or less, about 0.04 RU s⁻¹ or less, about 0.03 RU s⁻¹ or less, about 0.02 RU s⁻¹ or less, about 0.01 RU s⁻¹ or less, about 0.008 RU s⁻¹ or less, about 0.005 RU s⁻¹ or less, or about 0.001 RU s⁻¹ or less. In some embodiments, an anti-CD22, anti-CD33 or anti-CD74 bispecific antibody or antigen-binding fragment used in the methods disclosed herein dissociates from their corresponding human antigens with a kd of about 0.5 RU s⁻¹ or less. In some embodiments, the kd is about 0.2 RU s⁻¹ or less. In some embodiments, the kd is about 0.1 RU s⁻¹ or less. In some embodiments, the kd is about 0.08 RU s⁻¹ or less. In some embodiments, the kd is about 0.06 RU s⁻¹ or less. In some embodiments, the kd is about 0.05 RU s⁻¹ or less. In some embodiments, the kd is about 0.02 RU s⁻¹ or less. In some embodiments, the kd is about 0.01 RU s⁻¹ or less. In some embodiments, the kd is about 0.005 RU s⁻¹ or less.

In some embodiments, an anti-CD22, anti-CD33 or anti-CD74 bispecific antibody or antigen-binding fragment used in the methods disclosed herein dissociates from their corresponding human antigens with a kd with the range of 0.001-0.5 RU s⁻¹, 0.001-0.1 RUs 1, 0.001-0.05 RU s⁻¹, 0.005-0.5 RU s⁻¹, 0.005-0.1 RU s⁻¹, 0.01-0.5 RU s⁻¹, 0.01-0.1 RU s⁻¹, or 0.01-0.05 RU s⁻¹. In some embodiments, the kd is within the range of 0.005-0.05 RU s⁻¹. In some embodiments, the kd is within the range of 0.01-0.1 RU s 1. In some embodiments, the kd is within the range of 0.005-0.1 RU s⁻¹. In some embodiments, the kd is within the range of 0.01-0.5 RU s⁻¹. In some embodiments, the kd is within the range of 0.01-0.1 RU s⁻¹.

In some embodiments, an anti-CD22, anti-CD33 or anti-CD74 bispecific antibody or antigen-binding fragment used in the methods disclosed herein associates with their corresponding human antigens with a ka of 1.13×10⁷ RU S⁻¹ or higher, as determined by SPR (e.g., BIAcore). In some embodiments, an anti-CD22, anti-CD33 or anti-CD74 bispecific antibody or antigen-binding fragment used in the methods disclosed herein associates with their corresponding human antigens with a ka of about 1.0×10⁵ RU S⁻¹ or higher, about 5.0×10⁵ RU S⁻¹ or higher, about 1.0×10⁶ RU S⁻¹ or higher, about 5.0×10⁶ RU S⁻¹ or higher, about 1.0×10⁷ RU S⁻¹ or higher, about 2.0×10⁷ RU S⁻¹ or higher, about 3×10⁷ RU S⁻¹ or higher, about 4.0×10⁷ RU S⁻¹ or higher, about 5.0×10⁷ RU S⁻¹ or higher, about 1.0×10⁸ RU S⁻¹ or higher, about 5.0×10⁸ RU S⁻¹ or higher, or about 1.0×10⁹ RU S⁻¹ or higher. In some embodiments, the ka is about 1.0×10⁶ RU S⁻¹ or higher. In some embodiments, the ka is about 5.0×10⁶ RU S⁻¹ or higher. In some embodiments, the ka is about 1.0×10⁷ RU S⁻¹ or higher. In some embodiments, the ka is about of 2.0×10⁷ RU S⁻¹ or higher. In some embodiments, the ka is about 5.0×10⁷ RU S⁻¹ or higher. In some embodiments, the ka is about 1.0×10⁸ RU S⁻¹ or higher. In some embodiments, the ka is about 1.0×10⁹ RU S⁻¹ or higher.

In some embodiments, the ka is within the range of 1.0×10⁵-1.0×10⁶ RU S⁻¹, 1.0×10⁶-1.0×10⁷ RU S⁻¹, 5.0×10⁶-5.0×10⁷ RU S⁻¹, 1.0×10⁷-5.0×10⁷ RU S⁻¹, 1.0×10⁷-1.0×108 RU S⁻¹, or 1.0×10⁸-1.0×10⁹ RU S⁻¹. In some embodiments, the ka is within the range of 1.0×10⁶-1.0×10⁷ RU S⁻¹. In some embodiments, the ka is within the range of 5.0×10⁶-5.0×10⁷ RU S⁻¹. In some embodiments, the ka is within the range of 1.0×10⁷-5.0×10⁷ RU S⁻¹. In some embodiments, the ka is within the range of 1.0×10⁷-1.0×10⁸ RU S⁻¹. In some embodiments, the ka is within the range of 1.0×10⁸-1.0×10⁹ RU S⁻¹.

Epitope mapping is a method of identifying the binding site, region, or epitope on a target protein where an antibody binds. A variety of methods are known in the art for mapping epitopes on target proteins. These methods include mutagenesis, including but not limited to, shotgun mutagenesis, site-directed mutagenesis, and alanine scanning; domain or fragment scanning; peptide scanning (e.g., Pepscan technology); display methods (e.g., phage display, microbial display, and ribosome/mRNA display); methods involving proteolysis and mass spectroscopy; and structural determination (e.g., X-ray crystallography and NMR). In some embodiments, anti-CD22, anti-CD33 or anti-CD74 bispecific antibodies or antigen-binding fragments described herein are characterized by assays including, but not limited to, N-terminal sequencing, amino acid analysis, HPLC, mass spectrometry, ion exchange chromatography, and papain digestion.

The anti-CD22, anti-CD33 and/or anti-CD74 antibodies used in the methods disclosed herein can bind to a linear, continuous conformational, or discontinuous conformational epitope of their corresponding antigens. Antibodies with the same target but different epitope specificities will exhibit different properties such as affinity, rate of induced internalization, induction of downstream signaling pathways leading to anti-inflammatory or immunomodulatory response, etc. For illustrative purpose, the anti-CD22 antibodies SM03, SM06 and hLL2 will be used as an example. As described in Leung et al. (2015) Mabs 7 (1): 66-76; Zhao et al. (2014) PLOS ONE 9 (5): e96697; U.S. Pat. No. U.S. Pat. No. 9,371,396B2; and Chinese Pat. No. ZL201210286457.4; which are incorporated herein in their entirety by reference, SM03 and SM06 bind to the domain 2 of human CD22 with high affinity, specifically at the discontinuous conformational epitope encompassing the sequence ₁₆₁CLLNFSCYGYPIQ₁₇₃ (SEQ ID NO: 17) and ₁₉₈VFTRSELKFSPQWSHHGKIVTC ₂₁₉ (SEQ ID NO: 18) with affinity (Ka) in the range of 0.82×10⁹ M⁻¹. Binding of this conformational epitope can induce the rapid internalization of CD22 (e.g., 50% of the surface CD22 can be internalized within 10 minutes), promote the cis-trans conversion of the 2,6-sialic acid binding of CD22, and restores immunotolerance. As described in Ereno-Orbea J et. al. (2017) Molecular basis of human CD22 function and therapeutic targeting. Nat Commun. 8 (1): 764, Epratuzumab binds to domain 2 and 3 of CD22. From in-house data, Epratuzumab showed much slower internalization of CD22 as compared to SM03 and SM06.

Accordingly, the anti-CD22, anti-CD33 or anti-CD74 bispecific antibodies or antigen-binding fragments that can be used in the methods disclosed herein can bind to an epitope of the corresponding antigens that (a) induces internalization of the surface receptors/antigens and/or (b) suppresses microglia, astrocyte, oligodendrocyte, pericyte brain endothelial cell or other neuro-immunological cell activation which promote neuro-inflammation and/or (c) reduces pathogenic synaptic pruning by microglia, astrocyte or other neuro-immunological cell and/or (d) modulate antigen presentation by microglia, astrocyte, endothelial cell, or other neuro-immunological cell to infiltrating T cells and/or (e) promote remyelination of damaged myelin sheath by oligodendrocyte or other neuro-immunological cells . . .

In some embodiments, the anti-CD22, anti-CD33 or anti-CD74 bispecific antibodies or antigen-binding fragments that can be used in the methods disclosed herein can bind linear or conformational epitopes of the corresponding antigens with a K_{A} in the range of about 1-10×10⁹ M⁻¹. In some embodiments, an anti-CD22 antibody or antigen-binding fragment used in the methods disclosed herein binds this conformational epitope with a K_{A} of about 1×10⁷ M⁻¹ or more, about 1×10⁸ M⁻¹ or more, about 5×10⁸ M⁻¹ or more, about 1×10⁹ M⁻¹ or more, about 5×10⁹ M⁻¹ or more, about 1×10¹⁰ M⁻¹ or more, about 5×10¹⁰ M⁻¹ or more, about 1×10¹¹ M⁻¹ or more, about 5×10¹¹ M⁻¹ or more, or about 1×10¹² M⁻¹ or more. In some embodiments, the K_{A} is about 1×10⁷ M⁻¹ or more. In some embodiments, the K_{A} is about 5×10⁷ M⁻¹ or more. In some embodiments, the K_{A} is about 1×10⁸ M⁻¹ or more. In some embodiments, the K_{A} is about 5×10⁸ M⁻¹ or more. In some embodiments, the K_{A} is about 8×10⁸ M⁻¹ or more. In some embodiments, the K_{A} is about 1×10⁹ M⁻¹ or more. In some embodiments, the K_{A} is about 5×10⁹ M⁻¹ or more.

In some embodiments, an anti-CD22, anti-CD33 or anti-CD74 bispecific antibody or antigen-binding fragment used in the methods disclosed herein binds linear or conformational epitopes of the corresponding antigens with a K_{A} within the range of about 1×10⁶-1×10⁷ M⁻¹, 5×10⁶-5×10⁷ M⁻¹, 1×10⁷-1×10⁸ M⁻¹, 5×10⁷-5×10⁸ M⁻¹, 1×10⁸-5×10⁸ M⁻¹, 1×10⁸-1×10⁹ M⁻¹, 5×10⁸-1×10⁹ M⁻¹, 5×10⁸-5×10⁹ M⁻¹, 1×10⁹-1×10¹⁰ M⁻¹, 5×10⁹-5×10¹⁰ M⁻¹, 1×10¹⁰-1×10¹¹ M⁻¹, 5×10¹⁰-5×10¹¹ M⁻¹, 1×10¹¹-1×10¹² M⁻¹ or 5×10¹¹-5×10¹² M⁻¹. In some embodiments, the K_{A} is within the range of about 1×10⁶-1×10⁷ M⁻¹. In some embodiments, the K_{A} is within the range of about 1×10⁶ 1×10⁷ M⁻¹. In some embodiments, the K_{A} is within the range of about 1×10⁷-1×10⁸ M⁻¹. In some embodiments, the K_{A} is within the range of about 1×10⁸-1×10⁹ M⁻¹. In some embodiments, the K_{A} is within the range of about 5×10⁸-1×10⁹ M⁻¹. In some embodiments, the K_{A} is within the range of about 5×10⁸-5×10⁹ M⁻¹. In some embodiments, the K_{A} is within the range of about 1×10⁹-1×10¹⁰ M⁻¹.

In some embodiments provided herein are also antibodies or antigen-binding fragments that compete with the antibody or antigen-binding fragment provided above for binding to CD22 (e.g., human CD22), CD33 (e.g., human CD33) or CD74 (e.g., human CD74). Antibodies that “compete with another antibody for binding to a target” refer to antibodies that inhibit (partially or completely) the binding of the other antibody to the target. Whether two antibodies compete with each other for binding to a target, i.e., whether and to what extent one antibody inhibits the binding of the other antibody to a target, can be determined using known competition experiments, e.g., BIACORE® surface plasmon resonance (SPR) analysis. In some embodiments, an anti-CD22, anti-CD33 or anti-CD74 antibody or antigen-binding fragment competes with, and inhibits binding of another antibody or antigen-binding fragment to the corresponding CD22 (e.g., human CD22), CD33 (e.g., human CD33) or CD74 (e.g., human CD74) by at least 50%, 60%, 70%, 80%, 90% or 100%. Competition assays can be conducted as described, for example, in Ed Harlow and David Lane, Cold Spring Harb Protoc; 2006; doi: 10.HOI/pdb. prot4277 or in Chapter 11 of “Using Antibodies” by Ed Harlow and David Lane, Cold Spring Harbor Laboratory Press, Cold Spring Harbor, NY, USA 1999.

In some embodiments provided herein are bispecific antibodies or antigen-binding fragments that compete with SM03, SM06 or hLL2 for binding to human CD22. In some embodiments, provided herein are bispecific antibodies or antigen-binding fragments that compete with Gemtuzumab, HuMy9-6, Lintuzumab, for binding to human CD33. In some embodiments, provided herein are bispecific antibodies or antigen-binding fragments that compete with cLL1 or hLL1, for binding to human CD74. In some embodiments, the anti-CD22, anti-CD33 or anti-CD74 antibodies or antigen-binding fragments used in the methods disclosed herein compete for binding with their corresponding antigens with radiolabeled I¹²⁵-SM03, I¹²⁵-SM06 or I¹²⁵-hLL2 for binding to native CD22 on Ramos cell, a human Burkitt's lymphoma cell line; or with radiolabeled I¹²⁵-Gemtuzumab, I¹²⁵-HuMy9-6 or I¹²⁵-Lintuzumab for binding to native CD33 on THP-1, a human monocytic cell line; or with radiolabeled I¹²⁵-cLL1 or I¹²⁵-hLL1 for binding to native CD74 on K-562, a human myelogenous leukemia cell line with an EC50 in the range of 1 to 10 μg/ml or less, or about 1 g/ml or less. In some embodiments, the anti-CD22, anti-CD33 or anti-CD74 antibodies or antigen-binding fragments used in the methods disclosed herein compete for binding with their corresponding antigens with radiolabeled I¹²⁵-SM03, I¹²⁵-SM06 or I¹²⁵-hLL2 for binding to native CD22 on Ramos cell, a human Burkitt's lymphoma cell line; or with radiolabeled I¹²⁵-Gemtuzumab, I¹²⁵-HuMy9-6 or I¹²⁵-Lintuzumab for binding to native CD33 on THP-1, a human monocytic cell line; or with radiolabeled I¹²⁵-cLL1 or I¹²⁵-hLL1 for binding to native CD74 on K-562, a human myelogenous leukemia cell line with an EC50 of about 10 μg/ml or less, or about 1 μg/ml. In some embodiments, the anti-CD22, anti-CD33 or anti-CD74 antibodies or antigen-binding fragments used in the methods disclosed herein compete for binding with their corresponding antigens with radiolabeled I¹²⁵-SM03, I¹²⁵-SM06 or I¹²⁵-hLL2 for binding to native CD22 on Ramos cell, a human Burkitt's lymphoma cell line; or with radiolabeled I¹²⁵-Gemtuzumab, I¹²⁵-HuMy9-6 or I¹²⁵-Lintuzumab for binding to native CD33 on THP-1, a human monocytic cell line; or with radiolabeled I¹²⁵-cLL1 or I¹²⁵-hLL1 for binding to native CD74 on K-562, a human myelogenous leukemia cell line with an EC50 of about 0.1 μg/ml or less, about 0.2 μg/ml or less, about 0.5 μg/ml or less, about 0.8 μg/ml or less, about 1 μg/ml or less, about 2 μg/ml or less, about 5.0 μg/ml or less, about 8 μg/ml or less, about 10 μg/ml or less, or about 50 μg/ml or less. In some embodiments, the EC50 is about 0.1 μg/ml or less. In some embodiments, the EC50 is about 0.5 μg/ml or less. In some embodiments, the EC50 is about 1 μg/ml or less. In some embodiments, the EC50 is about 2 μg/ml or less. In some embodiments, the EC50 is about 5 μg/ml or less. In some embodiments, the EC50 is about 10 μg/ml or less. In some embodiments, the anti-CD22, anti-CD33 or anti-CD74 antibodies or antigen-binding fragments used in the methods disclosed herein compete for binding with their corresponding antigens with radiolabeled I¹²⁵-SM03, I¹²⁵-SM06 or I¹²⁵-hLL2 for binding to native CD22 on Ramos cell, a human Burkitt's lymphoma cell line; or with radiolabeled I¹²⁵-Gemtuzumab, I¹²⁵-HuMy9-6 or I¹²⁵-Lintuzumab for binding to native CD33 on THP-1, a human monocytic cell line; or with radiolabeled I¹²⁵-cLL1 or I¹²⁵-hLL1 for binding to native CD74 on K-562, a human myelogenous leukemia cell line with an EC₅₀ within the range of about 0.1-50 μg/ml, about 0.1-10 μg/ml, about 0.1-5 μg/ml, about 0.5-10 μg/ml, about 0.5-5 μg/ml, about 1-10 μg/ml or about 1-5 μg/ml. In some embodiments, the EC₅₀ is within the range of about 0.1-50 ng/ml. In some embodiments, the EC₅₀ is within the range of about 0.1-10 μg/ml. In some embodiments, the EC₅₀ is within the range of about 0.1-5 μg/ml. In some embodiments, the EC₅₀ is within the range of about 0.5-10 μg/ml. In some embodiments, the EC₅₀ is within the range of about 0.5-5 μg/ml. In some embodiments, the EC₅₀ is within the range of about 1-10 g/ml. In some embodiments, the EC₅₀ is within the range of about 1-5 μg/ml.

In some embodiments, the anti-CD22, anti-CD33 or anti-CD74 antibodies or antigen-binding fragments used in the methods disclosed herein compete for binding with their corresponding antigens with radiolabeled I¹²⁵-SM03, I¹²⁵-SM06 or I¹²⁵-hLL2; or with radiolabeled I¹²⁵-Gemtuzumab, I¹²⁵-HuMy9-6 or I¹²⁵-Lintuzumab; or with radiolabeled I¹²⁵-cLL1 or I¹²⁵-hLL1 for binding to their corresponding native antigens on a microglia cell (e.g., HMC-3 cell) or/and on an oligodendrocyte cell line (e.g. HOG or MO3.13 cell) with an EC₅% of about 0.1 μg/ml or less, about 0.2 μg/ml or less, about 0.5 μg/ml or less, about 0.8 μg/ml or less, about 1 μg/ml or less, about 2 μg/ml or less, about 5.0 μg/ml or less, about 8 μg/ml or less, about 10 μg/ml or less, or about 50 μg/ml or less. In some embodiments, the EC₅₀ is about 0.1 μg/ml or less. In some embodiments, the EC₅₀ is about 0.2 μg/ml or less. In some embodiments, the EC₅₀ is about 0.5 μg/ml or less. In some embodiments, the EC₅₀ is about 1 μg/ml or less. In some embodiments, the EC₅₀ is about 2 μg/ml or less. In some embodiments, the EC₅₀ is about 5 μg/ml or less. In some embodiments, the EC₅₀ is about 10 μg/ml or less. In some embodiments, the anti-CD22, anti-CD33 or anti-CD74 antibodies or antigen-binding fragments used in the methods disclosed herein compete for binding with their corresponding antigens with radiolabeled I¹²⁵-SM03, I¹²⁵-SM06 or I¹²⁵-hLL2; or with radiolabeled I¹²⁵-Gemtuzumab, I¹²⁵-HuMy9-6 or I¹²⁵-Lintuzumab; or with radiolabeled I¹²⁵-LL1 or I¹²⁵-hLL1 for binding to their corresponding native antigens on a microglia cell (e.g., HMC-3 cell) or/and on an oligodendrocyte cell line (e.g. HOG or MO3.13 cell) with an EC₅₀ within the range of about 0.1-50 μg/ml, about 0.1-10 μg/ml, about 0.1-5 μg/ml, about 0.5-10 μg/ml, about 0.5-5 μg/ml, about 1-10 μg/ml or about 1-5 μg/ml. In some embodiments, the EC₅₀ is within the range of about 0.1-50 μg/ml. In some embodiments, the EC₅₀ is within the range of about 0.1-10 ng/ml. In some embodiments, the EC₅₀ is within the range of about 0.1-5 μg/ml. In some embodiments, the EC₅₀ is within the range of about 0.5-10 μg/ml. In some embodiments, the EC₅₀ is within the range of about 0.5-5 μg/ml. In some embodiments, the EC₅₀ is within the range of about 1-10 g/ml. In some embodiments, the EC₅₀ is within the range of about 1-5 μg/ml.

In some embodiments, the anti-CD22, anti-CD33 or anti-CD74 antibodies or antigen-binding fragments provided herein can induce the internalization of about 20%, about 30%, about 40%, about 50%, about 60%, about 70% of the corresponding surface antigens (i.e. CD22, CD33 or CD74) within 10 minutes (min) upon contact with the cell (e.g., B cell, monocyte, macrophage, dendritic cell, oligodendrocyte or microglia cell). In some embodiments, the internalization rate is about 30% of the corresponding surface antigens (e.g. CD22, CD33 or CD74) within 10 min. In some embodiments, the internalization rate is about 40% of the corresponding surface antigens (e.g. CD22, CD33 or CD74) within 10 min. In some embodiments, the internalization rate is about 50% of the corresponding surface antigens (e.g. CD22, CD33 or CD74) within 10 min. In some embodiments, the internalization rate is about 60% of the corresponding surface antigens (e.g. CD22, CD33 or CD74) within 10 min. In some embodiments, the internalization rate is about 70% of the corresponding surface antigens (e.g. CD22, CD33 or CD74) within 10 min. In some embodiments, the anti-CD22, anti-CD33 or anti-CD74 antibodies or antigen-binding fragments provided herein can induce the internalization of about 20% to 70%, about 30% to 70%, about 40% to 70%, about 30% to 60%, or about 40% to 60% of the corresponding surface antigens (e.g. CD22, CD33 or CD74) within 10 min upon contact. In some embodiments, the internalization rate is about 20% to 70% of the surface antigens (e.g. CD22, CD33 or CD74) within 10 min. In some embodiments, the internalization rate is about 40% to 70% of the surface antigens (e.g. CD22, CD33 or CD74) within 10 min. In some embodiments, the internalization rate is about 30% to 60% of the corresponding surface antigens (e.g. CD22, CD33 or CD74) within 10 min. In some embodiments, the internalization rate is about 40% to 60% of the corresponding surface antigens (e.g. CD22, CD33 or CD74) within 10 min.

In some embodiments, the anti-CD22, anti-CD33 or anti-CD74 antibodies or antigen-binding fragments provided herein can induce the internalization of about 50% of the corresponding surface antigens (e.g. CD22, CD33 or CD74) within about 2 min, about 5 min, about 10 min, about 15 min, about 20 min, about 30 min, or about 1 hour upon contact with the cell (e.g., B cell, monocyte, macrophage, dendritic cell, oligodendrocyte or microglia cell). In some embodiments, 50% of the corresponding surface antigens (e.g. CD22, CD33 or CD74) is internalized within 2 min. In some embodiments, 50% of the corresponding surface antigens (e.g. CD22, CD33 or CD74) is internalized within 5 min. In some embodiments, 50% of the corresponding surface antigens (e.g. CD22, CD33 or CD74) is internalized within 10 min. In some embodiments, 50% of the corresponding surface antigens (e.g. CD22, CD33 or CD74) is internalized within 15 min. In some embodiments, 50% of the corresponding surface antigens (e.g. CD22, CD33 or CD74) is internalized within 20 min. In some embodiments, 50% of the corresponding surface antigens (e.g. CD22, CD33 or CD74) is internalized within 30 min.

In some embodiments, the anti-CD22, anti-CD33 or anti-CD74 bispecific antibodies or antigen-binding fragments that also exhibit binding specificities against Aβ protein provided herein can induce the internalization of Aβ at a rate of about 0.2 pg/s, about 0.5 pg/s, about 0.8 pg/s, about 1.0 pg/s, about 2.0 pg/s, about 5.0 pg/s, about 8.0 pg/s, about 10.0 pg/s, or about 10.0 pg/s. In some embodiments, the rate is about 0.2 pg/s. In some embodiments, the rate is about 0.5 pg/s. In some embodiments, the rate is about 0.8 pg/s. In some embodiments, the rate is about 1.0 pg/s. In some embodiments, the rate is about 2.0 pg/s. In some embodiments, the rate is about 2.0 pg/s. In some embodiments, the rate is about 5.0 pg/s. In some embodiments, the rate is about 1.0 pg/s. In some embodiments, the anti-CD22 antibodies or antigen-binding fragments provided herein can induce the internalization of Aβ at a rate of about 0.2-20 pg/s, about 0.2-10 pg/s, about 0.2-5 pg/s, about 0.2-2 pg/s, about 0.5-20 pg/s, about 0.5-10 pg/s, about 0.5-5 pg/s, about 0.5-2 pg/s, about 1-20 pg/s, about 1-10 pg/s, about 1-5 pg/s, or about 1-2 pg/s. In some embodiments, the rate is about 0.2-20 pg/s. In some embodiments, the rate is about 0.2-10 pg/s. In some embodiments, the rate is about 0.5-10 pg/s. In some embodiments, the rate is about 0.5-5 pg/s. In some embodiments, the rate is about. In some embodiments, the rate is about 1-2 pg/s.

In some embodiments, anti-CD22, anti-CD33 or anti-CD74 bispecific antibodies or antigen-binding fragments that also exhibit binding specificities against Aβ protein provided herein can be derivatized or linked to another functional molecule (e.g., another peptide or protein) and used in methods disclosed herein. Accordingly, in some embodiments, the antibodies and antigen-binding fragments used in methods disclosed herein include derivatized and otherwise modified forms of the bispecific antibodies described herein, including immunoadhesion molecules. For example, the antibodies and antigen-binding fragments can be functionally linked (by chemical coupling, genetic fusion, noncovalent association or introduction of an artificial amino acid/functional group suitable for site-specific conjugation) to one or more other molecular entities, such as another antibody, a detectable agent, a cytotoxic agent, a pharmaceutical agent, and/or a protein or peptide that can mediate associate of the antibody or antigen-binding fragment with another molecule (such as a streptavidin core region or a polyhistidine tag).

One type of derivatized antibody is produced by crosslinking two or more antibodies (of the same type or of different types, e.g., to create tri- or multi-specific antibodies). Suitable crosslinkers include those that are heterobifunctional, having two distinctly reactive groups separated by an appropriate spacer (e.g., m-malcimidobenzoyl-N-huydroxysuccinimide ester) or homobifunctional (e.g., disuccinimidyle suberate). Such linkers are available from Thermo Scientific, Waltham, MA.

In some embodiments, an anti-CD22, anti-CD33 or anti-CD74 bispecific antibody or antigen-binding fragment is conjugated to a cytotoxic agent or moiety. In some embodiments, an anti-CD22, anti-CD33 or anti-CD74 bispecific antibody or antigen-binding fragment is conjugated to a cytotoxic agent to form an ADC (antibody-drug conjugate). In some embodiments, the cytotoxic moiety is a chemotherapeutic agent including, but not limited to, methotrexate, adriamycin/doxorubicin, melphalan, mitomycin C, chlorambucil, duocarmycin, daunorubicin, pyrrolobenzodiazepines (PBDs), or other intercalating agents. In some embodiments, the cytotoxic moiety is a microtubule inhibitor including, but not limited to, auristatins, maytansinoids (e.g., DM1 and DM4), and tubulysins. In some embodiments, the cytotoxic moiety is an enzymatically active toxin of bacterial, fungal, plant, or animal origin, or fragments thereof, including, but not limited to, diphtheria A chain, non-binding active fragments of diphtheria toxin, exotoxin A chain, ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, cnomycin, and the tricothecenes. In some embodiments, an antibody is conjugated to one or more small molecule toxins, such as calicheamicins, maytansinoids, trichothenes, and CC1065.

In some embodiments, an anti-CD22, anti-CD33 or anti-CD74 bispecific antibody or antigen-binding fragment described herein is conjugated to a detectable substance or molecule that allows the agent to be used for diagnosis and/or detection. A detectable substance can also include, but is not limited to, enzymes, such as horseradish peroxidase, alkaline phosphatase, glucose oxidase, beta-galactosidase, and acetylcholinesterase; prosthetic groups, such as biotin and flavine(s); fluorescent materials, such as, umbelliferone, fluorescein, fluorescein isothiocyanate (FITC), rhodamine, tetramethylrhodamine isothiocyanate (TRITC), dichlorotriazinylamine fluorescein, dansyl chloride, cyanine (Cy3), 5-dimethylamine-1-napthalenesulfonyl chloride, and phycoerythrin; bioluminescent materials, such as luciferase; radioactive materials, such as ²¹²Bi, ¹⁴C, ⁵⁷Co, ⁵¹Cr, ⁶⁷Cu, ¹⁸F, ⁶⁸Ga, ⁶⁷Ga, ¹⁵³Gd, ¹⁵⁹Gd, ⁶⁸Ge, ³H, ¹⁶⁶Ho, ¹³¹I, ¹²⁵I, ¹²³I, ¹²¹I, ¹¹⁵In, ¹¹³In, ¹¹²In, ¹¹¹In, ¹⁴⁰La, ¹⁷⁷Lu, ⁵⁴Mn, ⁹⁹Mo, ³²P, ¹⁰³Pd, ¹⁴⁹Pm, ¹⁴²Pr, ¹⁸⁶Re, ¹⁸⁸Rc, ¹⁰⁵Rh, ⁹⁷Ru, ³⁵S, ⁴⁷Sc, ⁷⁵Sc, ¹⁵³Sm, ¹¹³Sn, ¹¹⁷Sn, ⁸⁵Sr, ^{99m}Tc, ²⁰¹Ti, ¹³³Xe, ⁹⁰Y, ⁶⁹Yb, ¹⁷⁵Yb, ⁶⁵Zn; positron emitting metals; and magnetic metal ions positron emitting metals; and magnetic metal ions.

An anti-CD22, anti-CD33 or anti-CD74 bispecific antibody or antigen-binding fragment described herein can be attached to a solid support. Such solid supports include, but are not limited to, glass, cellulose, polyacrylamide, nylon, polystyrene, polyvinyl chloride, or polypropylene. In some embodiments, an immobilized anti-CD22, anti-CD33 or anti-CD74 bispecific antibody or antigen-binding fragment is used in an immunoassay. In some embodiments, an immobilized anti-CD22, anti-CD33 or anti-CD74 bispecific antibody or antigen-binding fragment is used in purification of the corresponding target antigen (e.g., human CD22, human CD33 or human CD74).

### 7.2.2 Additional CD22 Antibodies

Disclosed herein are methods of promoting removal of Aβ plaque, methods of reducing neuroinflammation, methods of preventing synaptic phagocytosis and/or neuronal death, methods of treating an Aβ-related disease or disorder, and methods of treating a disease or disorder associated with neuroinflammation using a therapeutically effective amount of an anti-CD22 (e.g., human CD22), anti-CD33 (e.g., human CD33) or anti-CD74 (e.g., human CD74) bispecific antibody or antigen-binding fragment disclosed herein, or their variants or equivalents. Without being bound by theory, it is to be understood that the anti-CD22, anti-CD33 or anti-CD74 bispecific antibodies disclosed herein can be used to achieve the above-mentioned therapeutic effect because they have at least one of the following functions or activities: (1) binding to an epitope, linear or conformational that promotes rapid internalization in microglia cells; (2) promotes removal of extracellular Aβ captured by the anti-Aβportion of the bispecific antibody and internalization of the captured Aβ protein in microglia cells (3) suppressing pro-inflammatory cytokines, such as NFκB signaling, and IL-6 secretion; and (4) reducing neuroinflammation.

It is to be understood that the anti-CD22, anti-CD33 or anti-CD74 bispecific antibodies or antigen-binding fragments can promote rapid internalization of their corresponding antigens on microglia cells. The rapid internalization of these corresponding antigens then leads to rapid internalization of both bispecific antibody-bound Aβ and soluble Aβ by microglia cells without engaging FcγR on the cell surface, thereby avoiding ARIA-E and ARIA-H that result from the FcγR involvement. Additionally, both the internalization of CD22, CD33 or CD74 bispecific antibody that also bind to Aβ can result in the removal of Aβ as well as the suppression of inflammatory cytokines, such as NFκB signaling, and IL-6 secretion, leading to the reduction of neuroinflammation.

As such, provided herein are also methods for selecting for bispecific antibodies or antigen-binding fragments with one specificity targeting specifically to CD22 (e.g., human CD22), CD33 (e.g., human CD33) or CD74 (e.g., human CD74) and the other specificity targeting Aβ protein for at least one of the following functions or activities, wherein the selected bispecific antibody or antigen-binding fragment can be used in methods of promoting the removal of Aβ plaque, methods of reducing neuroinflammation, methods of preventing synaptic phagocytosis and/or neuronal death, methods of treating an Aβ-related disease or disorder, and/or methods of treating a disease or disorder associated with neuroinflammation: (1) binding to an epitope that promote antigen internalization on cells that express the respective antigens on the cell surface, e.g., microglia cells, Ramos cell, and/or B cell, optionally at a rate of 50% antigen internalized by 10 minutes; (2) promoting internalization of Aβ in microglia cells, optionally at a rate of 1.76 pg/s; (3) reducing neuroinflammation; and (4) suppressing pro-inflammatory cytokines, such as NFκB signaling, and IL-6 secretion. In some embodiments, the selected antibodies or antigen-binding fragments can be used in methods of promoting the removal of Aβ plaque. In some embodiments, the selected antibodies or antigen-binding fragments can be used in methods of reducing neuroinflammation. In some embodiments, the selected antibodies or antigen-binding fragments can be used in methods of preventing synaptic phagocytosis and/or neuronal death. In some embodiments, the selected antibodies or antigen-binding fragments can be used in methods of treating an Aβ-related disease or disorder. In some embodiments, the selected antibodies or antigen-binding fragments can be used in methods of treating a disease or disorder associated with neuroinflammation.

In some embodiments, antibodies or antigen-binding fragments thereof that specifically bind to linear, continuous conformation or discontinuous conformation epitope in different extracellular domains of the respective antigens, such as CD22 (e.g., human CD22), CD33 (e.g., human CD33), or CD74 (e.g., human CD74) induce rapid internalization as disclosed herein can be isolated by screening of a recombinant combinatorial antibody library, preferably a scFv phage display library, prepared using human VL and VH cDNAs prepared from mRNA derived from human lymphocytes. Methodologies for preparing and screening such libraries are known in the art. In addition to commercially available kits for generating phage display libraries (e.g., the GE Healthcare Life Sciences Recombinant Antibody Phage System (RAPS); and the New England Biolab Ph. DTM Phage Display Library Kit, catalog no. E8100S), examples of methods and reagents particularly amenable for use in generating and screening antibody display libraries can be found in, for example, Ladner et al. U.S. Pat. No. 5,223,409; Fuchs et al. (1991) Biotechnology 9:1369-1372; Hay et al. (1992) Hum Antibod Hybridomas 3:81-85; Huse et al. (1989) Science 246:1275-1281; McCafferty et al., Nature (1990) 348:552-554; Griffiths et al. (1993) EMBO J 12:725-734; Hawkins et al. (1992) J Mol Biol 226:889-896; Clackson et al. (1991) Nature 352:624-628; Gram et al. (1992) PNAS 89:3579-3580; Garrard et al. (1991) Biotechnology 9:1373-1377; Hoogenboom et al. (1991) Nuc Acid Res 19:4133-4137; and Barbas et al. (1991) PNAS 88:7978-7982.

In some embodiments, the anti-CD22, anti-CD33 or anti-CD74 bispecific antibody or antigen-binding fragment to be used in the methods disclosed herein is further selected for binding to their corresponding antigens with a Ka of 0.1×10⁹ M⁻¹ or higher. In some embodiments, the anti-CD22, anti-CD33 or anti-CD74 bispecific antibody or antigen-binding fragment is selected for binding to their corresponding antigens with a K_{D} of about 1 nM or lower. In some embodiments, the anti-CD22, anti-CD33 or anti-CD74 bispecific antibody or antigen-binding fragment to be used in the methods disclosed herein is selected for dissociating from their corresponding antigens with a kd of 0.1 RU s⁻¹ or less, or a kd of 0.01 RU s⁻¹ or less. The binding parameters mentioned herein can be determined by any methods disclosed herein or known in the art, for example, by surface plasmon resonance.

In some embodiments, to isolate murine, recombinant or human antibodies with high affinity and a low off rate constant for human CD22, human CD33 or CD74, a parent murine or chimeric anti-CD22, anti-CD33, or anti-CD74 antibody having high affinity and a low off rate constant for human CD22 is first used to select human heavy and light chain sequences having similar binding activity toward the respective and corresponding antigens, using the epitope imprinting methods described in PCT Publication No. WO 93/06213. The antibody libraries used in this method are preferably scFv libraries prepared and screened as described in PCT Publication No. WO 92/01047, McCafferty et al., Nature (1990) 348:552-554; and Griffiths et al., (1993) EMBO J 12:725-734. The scFv antibody libraries preferably are screened using recombinant human CD22, human CD33 or human CD74 and/or recombinant fusion proteins with the extracellular domains of the respective and corresponding antigens fused to the Fc portion of a murine or human immunoglobulin.

Once initial human VL and VH segments are selected, “mix and match” experiments, in which different pairs of the initially selected VL and VH segments are screened for human CD22, human CD33, or human CD74 binding, are performed to select preferred VL/VH pair combinations. The affinity and/or specificity of binding of an antibody for a particular antigen can be increased using methods of “directed evolution,” as described by Wu et al., J. Mol. Biol., 294:151 (1999), the contents of which are incorporated herein by reference herein in their entirety. Additionally, to further improve the affinity and/or lower the off-rate constant for human CD22, human CD33 or huma CD74 binding, the VL and VH segments of the preferred VL/VH pair(s) can be randomly mutated, preferably within the CDR3 region of VH and/or VL, in a process analogous to the in vivo somatic mutation process responsible for affinity maturation of antibodies during a natural immune response. This in vitro affinity maturation can be accomplished by amplifying VH and VL regions using PCR primers complimentary to the VH CDR3 or VL CDR3, respectively, which primers have been “spiked” with a random mixture of the four nucleotide bases at certain positions such that the resultant PCR products encode VH and VL segments into which random mutations have been introduced into the VH and/or VL CDR3 regions. These randomly mutated VH and VL segments can be rescreened for binding to the extracellular domains of human CD22, human CD33, or human CD74, and/or recombinant fusion proteins with the extracellular domains of the respective and corresponding antigens fused to the Fc portion of a murine or human immunoglobulin.

In addition to phage display, candidate monoclonal antibodies for screening can also be prepared using hybridoma methods, whin are well known to one of skill in the art. For example, using a hybridoma method, a mouse, rat, rabbit, hamster, or other appropriate host animal, is immunized as described above. In some embodiments, lymphocytes are immunized in vitro. In some embodiments, the immunizing antigen is a human protein or a fragment thereof. In some embodiments, the immunizing antigen is a human protein or a fragment thereof.

Following immunization, lymphocytes are isolated and fused with a suitable myeloma cell line using, for example, polyethylene glycol. The hybridoma cells are selected using specialized media as known in the art and unfused lymphocytes and myeloma cells do not survive the selection process. Hybridomas that produce monoclonal antibodies directed to a chosen antigen can be identified by a variety of methods including, but not limited to, immunoprecipitation, immunoblotting, and in vitro binding assays (e.g., flow cytometry, FACS, ELISA, BLI, SPR (e.g., Biacore), and radioimmunoassay). Once hybridoma cells that produce antibodies of the desired specificity, affinity, and/or activity are identified, the clones may be subcloned by limiting dilution or other techniques. The hybridomas can be propagated either in in vitro culture using standard methods or in vivo as ascites tumors in an animal. The monoclonal antibodies can be purified from the culture medium or ascites fluid according to standard methods in the art including, but not limited to, affinity chromatography, ion-exchange chromatography, gel electrophoresis, and dialysis.

Often, framework residues in the framework regions can be substituted with the corresponding residue from the CDR donor antibody to alter, preferably improve, antigen binding. These framework substitutions are identified by methods well-known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature, 332:323, which are incorporated herein by reference in their entireties.)

A humanized antibody has one or more amino acid residues introduced into it from a source which is nonhuman. These nonhuman amino acid residues are often referred to as “import” residues, which are typically taken from an “import” variable domain. Thus, humanized antibodies comprise one or more CDRs from nonhuman immunoglobulin molecules and framework regions from human. Methods of humanization of antibodies are well-known in the art, including CDR-grafting (see, e.g., Jones et al., Nature, 321:522-525 (1986); Riechmann et al., Nature, 332:323-327 (1988); Verhocyen et al., Science, 239:1534-1536 (1988); European Patent No. EP 239, 400; International Publication No. WO 91/09967; and U.S. Pat. Nos. 4,816,567; 6,331,415; 5,225,539; 5,530,101; 5,585,089; and 6,548,640; each of which is incorporated herein in its entirety by reference). In such humanized antibodies, substantially less than an intact human variable domain has been substituted by the corresponding sequence from a nonhuman species. In practice, humanized antibodies are typically human antibodies in which some CDR residues and possibly some FR residues are substituted by residues from analogous sites in rodent antibodies. Methods of producing humanized antibodies also include veneering or resurfacing (see, e.g., European Patent Nos. EP 592, 106 and EP 519, 596; Padlan, 1991, Molecular Immunology, 28 (4/5): 489-498; Studnicka et al., 1994, Protein Engineering, 7 (6): 805-814; and Roguska et al., 1994, PNAS, 91:969-973, each of which is incorporated herein by its entirety by reference), chain shuffling (see, e.g., U.S. Pat. No. 5,565,332, which is incorporated herein in its entirety by reference), and techniques disclosed in, e.g., U.S. Patent Application Publication No. US2005/0042664, U.S. Patent Application Publication No. US2005/0048617, U.S. Pat. Nos. 6,407,213, 5,766,886, International Publication No. WO 9317105, Tan et al., J. Immunol., 169:1119-25 (2002), Caldas et al., Protein Eng., 13 (5): 353-60 (2000), Morea et al., Methods, 20 (3): 267-79 (2000), Baca et al., J. Biol. Chem., 272 (16): 10678-84 (1997), Roguska et al., Protein Eng., 9 (10): 895-904 (1996), Couto et al., Cancer Res., 55 (23 Supp): 5973s-5977s (1995), Couto et al., Cancer Res., 55 (8): 1717-22 (1995), Sandhu J S, Gene, 150 (2): 409-10 (1994), and Pedersen et al., J. Mol. Biol., 235 (3): 959-73 (1994), each of which is incorporated herein in its entirety by reference.

The choice of human variable domains, both light and heavy, to be used in making the humanized antibodies is to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151:2296 (1993); Chothia et al., J. Mol. Biol., 196:901 (1987), the contents of which are incorporated herein by reference herein in their entirety). Another method uses a particular framework derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993), the contents of which are incorporated herein by reference herein in their entirety).

Antibodies can be humanized with retention of high affinity for the target antigen and other favorable biological properties. For example, humanized antibodies can be prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind the target antigen. In this way, FR residues can be selected and combined from the recipient and import sequences so that the desired antibody characteristic, such as increased affinity for the target antigen, is achieved. In general, the CDR residues are directly and most substantially involved in influencing antigen binding.

Following screening and isolation of an anti-CD22, anti-CD33, or anti-CD74 antibody disclosed herein from a recombinant immunoglobulin display library, nucleic acid encoding the selected antibody can be recovered from the display package (e.g., from the phage genome) and subcloned into other expression vectors by standard recombinant DNA techniques. If desired, the nucleic acid can be further manipulated to create other antibody forms disclosed herein or known in the art (e.g., linked to a nucleic acid encoding additional immunoglobulin domains, such as additional constant regions). Methods to express a recombinant human antibody isolated by screening of a combinatorial library, including cloning the DNA encoding the antibody into a recombinant expression vector and introduced into a mammalian host cell, are well known in the art.

In some embodiments, the anti-CD22, anti-CD33, or anti-CD74 antibody or antigen-binding fragment is selected for (1) binding to an epitope, linear or conformational that promotes rapid internalization in microglia cells; (2) promotes removal of extracellular Aβ captured by the anti-Aβportion of the bispecific antibody and internalization of the captured Aβ protein in microglia cells, preferably without engaging FcγR on the cell surface (3) suppressing pro-inflammatory cytokines, such as NFκB signaling, and IL-6 secretion; and (4) reducing neuroinflammation by microglia cells, or any combination thereof.

In some embodiments, the anti-CD22, anti-CD33 or anti-CD74 antibody or antigen-binding fragment is selected for its function of promoting internalization of the respective and corresponding antigens. In some embodiments, the anti-CD22, anti-CD33 or anti-CD74 antibody or antigen-binding fragment is selected for its function of promoting internalization of Aβ in microglia cells, preferably without engaging FcγR on the cell surface. In some embodiments, the anti-CD22, anti-CD33 or anti-CD74 antibody or antigen-binding fragment is selected for its function of reducing neuroinflammation. In some embodiments, the anti-CD22, anti-CD33 or anti-CD74 antibody or antigen-binding fragment is selected for its function of suppressing NFκB signaling in microglia cells. In some embodiments, the anti-CD22, anti-CD33 or anti-CD74 antibody or antigen-binding fragment is selected for its function of suppressing IL-6 secretion by microglia cells. In some embodiments, the anti-CD22, anti-CD33 or anti-CD74 antibody or antigen-binding fragment is selected for its function of suppressing neuroinflammation. In some embodiments, the anti-CD22, anti-CD33 or anti-CD74 antibody or antigen-binding fragment is selected for its function of suppressing synaptic phagocytosis. In some embodiments, the anti-CD22, anti-CD33 or anti-CD74 antibody or antigen-binding fragment is selected for its function of suppressing neuronal death.

Anti-CD22, anti-CD33 or anti-CD74 antibodies or antigen-binding fragments described herein can be tested for binding to their respective and corresponding antigens, namely, human CD22, human CD33 or human CD74 by, for example, standard ELISA. Briefly, microtiter plates are coated with the respective and corresponding purified antigens, namely, CD22, CD33 or CD74, and then blocked with bovine serum albumin. Dilutions of antibody (e.g., dilutions of plasma from antigen-immunized mice) are added to each well and incubated. The plates are washed and incubated with secondary reagent (e.g., for human antibodies, a goat-anti-human IgG Fc-specific polyclonal reagent) conjugated to horseradish peroxidase (HRP). After washing, the plates can be developed and analyzed by a spectrophotometer. Sera from immunized mice can then be further screened by flow cytometry for binding to a cell line expressing human CD22, human CD33 or CD74, but not to a control cell line that does not the respective and corresponding antigens. Briefly, the binding of anti-CD22, anti-CD33 or anti-CD74 antibodies can be assessed by incubating recombinant CHO cells expressing the respective antigens with the corresponding anti-CD22, anti-CD33 or anti-CD74 antibody. The cells can be washed and binding can be detected with an anti-human IgG Ab. Flow cytometric analyses can be performed using a FACScan flow cytometry (Becton Dickinson, San Jose, CA). Mice which develop the highest titers can be used for fusions.

An ELISA assay as described above can be used to screen for antibodies and, thus, hybridomas that produce antibodies that show positive reactivity with the respective and corresponding immunogens for the anti-CD22, anti-CD33 or anti-CD75 antibody as disclosed herein. Hybridomas that produce antibodies that bind with high affinity to CD22, CD33, or CD74 can then be subcloned and further characterized. One clone from each hybridoma, which retains the reactivity of the parent cells (by ELISA), can then be chosen for making a cell bank, and for antibody purification.

To purify anti-CD22, anti-CD33 or anti-CD74 antibodies, selected hybridomas can be grown for monoclonal antibody purification. Supernatants can be filtered and concentrated before affinity chromatography. Eluted IgG can be checked by gel electrophoresis and high-performance liquid chromatography to ensure purity. The buffer solution can be exchanged, and the concentration can be determined. The monoclonal antibodies can be aliquoted and stored.

A variety of methods and assays are known in the art to determine the structural and functional properties of the antibodies and antigen-binding fragments. For example, to determine if the selected anti-CD22, anti-CD33 or anti-CD74 monoclonal antibodies bind to unique epitopes, each antibody can be biotinylated using commercially available reagents (Pierce, Rockford, IL). Biotinylated MAb binding can be detected with a streptavidin labeled probe. Competition studies using unlabeled monoclonal antibodies and biotinylated monoclonal antibodies can be performed using ELISA plates coated with the respective and corresponding CD22, CD33 or CD74 antigens.

To test the binding of monoclonal antibodies to live cells expressing CD22, CD33, or CD74, flow cytometry can be used. Briefly, cell lines expressing the respective and corresponding antigens, namely, CD22, CD33, or CD74, (grown under standard growth conditions) are mixed with various concentrations of monoclonal antibodies in PBS containing 0.1% BSA at 4° C. for 1 hour. After washing, the cells are reacted with Fluorescein-labeled anti-IgG antibody under the same conditions as the primary antibody staining. The samples can be analyzed by FACScan instrument using light and side scatter properties to gate on single cells and binding of the labeled antibodies is determined. An alternative assay using fluorescence microscopy can be used (in addition to or instead of) the flow cytometry assay. Cells can be stained exactly as described above and examined by fluorescence microscopy. This method allows visualization of individual cells, but can have diminished sensitivity depending on the density of the antigen.

Methods for analyzing binding affinity, cross-reactivity, and binding kinetics of various anti-CD22, anti-CD33 or anti-CD74 antibodies include standard assays known in the art, for example, biolayer interferometry (BLI) using, for example, Gator system (Probe Life) or the Octet-96 system (Sartorius AG), or BIACORE™ surface plasmon resonance (SPR) analysis using a BIACORE™ 2000 SPR instrument (Biacore Aβ, Uppsala, Sweden).

Assays to test or screen anti-CD22, anti-CD33 or anti-CD74 antibodies or antigen-binding fragments for functional properties disclosed above are well known in the art. Any methods disclosed herein or otherwise known in the art can be used in the methods of screening disclosed herein. For example, the functional assays that can be used in the screening methods disclosed herein include the luciferase assay for measuring NFκB signaling, the ELISA assay for measuring IL-6 release, the ELISA or Western Blot assay for measuring synaptic phagocytosis, and the MTT assay for measuring neuronal death, all of which are disclosed in the experimental section below. In some embodiments, provided herein are also the anti-CD22, anti-CD33 or anti-CD74 bispecific antibodies and antigen-binding fragments identified or produced using the methods described herein, as well as their therapeutic uses.

### 7.3 Methods of Production

The anti-CD22, anti-CD33 or anti-CD74 bispecific antibodies and antigen-binding fragments thereof that that can be used in methods disclosed herein, including but not limited to monoclonal antibodies, chimeric antibodies, human antibodies, and humanized antibodies, can be prepared by any methods disclosed herein or otherwise known in the art.

Methods of antibody production are well-known in the art. See for example, in Harlow et al., ANTIBODIES: A LABORATORY MANUAL, (Cold Spring Harbor Laboratory Press, 2nd ed. 1988); Hammerling et al., in: Monoclonal Antibodies and T-Cell Hybridomas 563, 681 (Elsevier, N. Y., 1981), each of which is incorporated herein in its entirety by reference.

In some embodiments, the antibodies or antigen-binding fragments that can be used in methods provided herein are recombinant, namely, prepared, expressed, produced or isolated by recombinant means. In some embodiments, the antibodies or antigen-binding fragments disclosed herein can be prepared, for example, by introducing recombinant expression vectors into host cells, a recombinant, combinatorial human antibody library, antibodies isolated from an animal (e.g., a mouse) that is transgenic for human immunoglobulin genes (see e.g., Taylor, L. D., et al. (1992) Nucl. Acid Res. 20:6287-95) or antibodies prepared, expressed, produced, or isolated by any other means that involves splicing of human immunoglobulin gene sequences to other DNA sequences.

In some embodiments, antibodies and antigen-binding fragments can be prepared by recombinant expression of immunoglobulin light and heavy chain genes in a host cell. To express an antibody recombinantly, a host cell is introduced with one or more recombinant expression vectors carrying DNA fragments encoding the immunoglobulin light and heavy chains of the antibody such that the light and heavy chains are expressed in the host cell and, preferably, secreted into the medium in which the host cells are cultured, from which medium the antibodies can be recovered. Standard recombinant DNA methodologies are used to obtain antibody heavy and light chain genes, incorporate these genes into recombinant expression vectors and introduce the vectors into host cells, such as those described in Sambrook, Fritsch and Maniais (cds), MOLECULAR CLONING: A LABORATORY MANUAL, Second Edition, Cold Spring Harbor, N.Y., (1989), Ausubel et al. (eds.) CURRENT PROTOCOLS IN MOLECULAR BIOLOGY, Greene Publishing Assoviates, (1989) and in U.S. Pat. No. 4,816,397.

To express a recombinant antibody or antigen fragment, DNA fragments encoding the light and heavy chain variable regions are first obtained. These DNAs can be obtained by amplification and modification of hybridomas for the murine antibody light and heavy chain variable sequences using the polymerase chain reaction (PCR), or by oligosynthesis based on the encoded amino acid sequence of design light and heavy chain variable sequences using standard methods known to those skilled in the art. The encoding DNA sequences can be further optimized to facilitate mammalian expression of the resultant antibody.

Once the VH and VL fragments for the murine antibody are obtained, these sequences can be mutated to encode the framework-patched version, the method of which was described in Chinese Pat. Nos. 01144894.6 and 031 23054.7 and U.S. Pat. No. 7,321,026 B2 &7,338,659 B2, both incorporated herein in their entirety by reference.

Once DNA fragments encoding VH and VL segments of the anti-CD22, anti-CD33 or anti-CD74 antibody are obtained (by, e.g., amplification and mutagenesis of the original murine VH and VL genes, as described above), these DNA fragments can be further manipulated by standard recombinant DNA techniques, for example to convert the variable region genes to full-length antibody chain genes, to Fab fragment genes or to a scFv gene. In these manipulations, a VL- or VH-encoding DNA fragment is operatively linked to another DNA fragment encoding another protein, such as an antibody constant region or a flexible linker. The term “operatively linked,” as used in this context, is intended to mean that the two DNA fragments are joined such that the amino acid sequences encoded by the two DNA fragments remain in-frame.

In some embodiments, the anti-CD22, anti-CD33 or anti-CD74 bispecific antibodies are operationally linked to antibodies or antibody fragments that are specific for the Aβ protein. Similarly, DNA fragments encoding VH and VL segments of the Aβ antibody are obtained (by, e.g., amplification and mutagenesis of the original murine VH and VL genes, as described above), these DNA fragments can be further manipulated by standard recombinant DNA techniques, for example to convert the variable region genes to full-length antibody chain genes, to Fab fragment genes or to a scFv gene. In these manipulations, a VL- or VH-encoding DNA fragment is operatively linked to another DNA fragment encoding another protein, such as an anti-CD22, anti-CD33 or anti-CD74 antibody via a flexible linker.

The isolated DNA encoding the VH region can be converted to a full-length heavy chain gene by operatively linking the VH-encoding DNA to another DNA molecule encoding heavy chain constant regions (CH1, CH2 and CH3). In some embodiments, the ant-CD22, anti-CD33 or anti-CD74 antibody is operationally linked to an Aβ antibody, preferably in the form of scFv, linked via a flexible linker at the C-terminal end of the anti-CD22, anti-CD33 or anti-CD74 antibody at the CH3 domain. The sequences of human heavy chain constant region genes are known in the art (see e.g., Kabat, E. A., et al (1991) SEQUENCES OF PROTEINS OF IMMUNOLOGICAL INTEREST, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, Ig4, IgA, IgE, IgM or IgD constant region, but most preferably is an IgG1 or IgG4 constant region. For a Fab fragment heavy chain gene, the VH-encoding DNA can be operatively linked to another DNA molecule encoding only the heavy chain CHI constant region. A scFv encoding DNA can further be operatively linked to the CHI constant region of the Fab fragment to generate a bispecific Fab.

The isolated DNA encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (see e.g., Kabat, E. A., et al (1991) SEQUENCES OF PROTEINS OF IMMUNOLOGICAL INTEREST, Fifth Edition, U.S. Department of Health and Human Services, NIH Publication No. 91-3242) and DNA fragments encompassing these regions can be obtained by standard PCR amplification. The light chain constant region can be a kappa or lambda constant region, but most preferably is a kappa constant region.

To prepare a scFv gene, the VH- and VL-encoding DNA fragments are operatively linked to another fragment encoding a flexible linker, e.g., encoding the amino acid sequence (Gly4-Ser) 3, such that the VH and VL sequences can be expressed as a contiguous single-chain protein, with the VL and VH regions joined by the flexible linker (see e.g., Bird et al. (1988) Science 242:423-426; Huston et al. (1988) Proc. Natl. Acad. Sci. USA 85:5879-5883; McCafferty et al. (1990) Nature 348:552-554).

To express the antibodies, or antigen-binding fragments that can be used in the methods disclosed herein, DNAs encoding partial or full-length light and heavy chains, obtained as described above, are inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present). In some embodiments, prior to insertion of the light or heavy chain sequences of an anti-CD22, anti-CD33 or anti-CD74 antibody, the expression vector already carries antibody constant regions sequences. In some embodiments, the expression vector carrying the antibody constant region sequences contains the scFv sequence derived from an anti-Aβantibody operationally linked to the CH3 domain sequence. For example, one approach to convert the VH and VL sequences of the anti-CD22, anti-CD33 or anti-CD74 antibody to full-length antibody genes is to insert them into expression vectors already encoding heavy chain constant and light chain constant regions, respectively, such that the VH segment is operatively linked to the CH segment(s) within the vector and the VL segment is operatively linked to the CL segment within the vector. DNA sequence encoding scFv of an Aβ antibody can be operationally linked to the CH3 segment sequence of the above expression vector using standard molecular biology methods to generate a bispecific antibody expression vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors provided herein can carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel; GENE EXPRESSION TECHNOLOGY: METHODS IN ENZYMOLOGY 185, Academic Press, San Diego, Calif. (1990). As those skilled in the art would appreciate, the design of the expression vector, including the selection of regulatory sequences depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from immunoglobulin heavy chain (IgH) enhancer (Gillies et al. (1983) Cell 33:717-728) metallothioneine (MT), cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. For further description of viral regulatory elements, and sequences thereof, see e.g., U.S. Pat. Nos. 5,665,578; 5,168,062; 4,510,245; and 4,968,615.

In addition to the antibody chain genes and regulatory sequences, the recombinant expression vectors provided herein can carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr-host cells with methotrexate selection/amplification), Glutamate Synthase (GS) gene and the neo gene (for G418 selection).

For expression of the light and heavy chains, the expression vector encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection, lipofection, protoplast fusion and the like. Although antibodies and antigen-binding fragments can be produced in either prokaryotic or eukaryotic host cells, expression of antibodies in eukaryotic cells, especially mammalian host cells, is preferred because such host cells are more likely than prokaryotic cells to assemble and secrete a properly folded and immunologically active antibody.

Preferred mammalian host cells for expressing the recombinant antibodies used in methods described herein include SP2/0 myeloma cells, NSO myeloma cells, COS cells, and Chinese Hamster Ovary (CHO cells) (including dfhr-CHO cells, described in Urlaub and Chasin, (1980) Proc. Natl. Acad. Sci. USA 77:4216-4200, used with a DHFR selectable marker, e.g., as described in R.J. Kaufman and P. A. Sharp (1982) J. Mol. Biol. 159:601-621). When recombinant antibody-encoding expression vectors are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Host cells can also be used to produce portions of intact antibodies, such as Fab fragments or scFv molecules. Expressly contemplated herein are variations of the above procedure. For example, it can be desirable to transfect a host cell with DNA encoding either the light chain or the heavy chain (but not both) of an antibody used in methods disclosed herein. Recombinant DNA technology can also be used to remove some or all of the DNA encoding either or both of the light and heavy chains that is not necessary for binding to respective and corresponding surface receptors/antigens, namely, CD22, CD33 or CD74 for surface antigen/receptor specificity, and Aβ for amyloid protein specificity. The molecules expressed from such truncated DNA molecules are also encompassed by the antibodies provided herein. Alternatively, bifunctional antibodies can be produced in which one heavy and one light chain are an antibody that specifically binds human surface receptor/antigen, namely, CD22, CD33, or CD74 and the other heavy and light chain are specific for a different antigen such as different forms of amyloid β protien by crosslinking an antibody of the invention to a second antibody by standard chemical crosslinking methods.

In some embodiments of the systems of recombinant expression of an antibody or antigen-binding fragment thereof that can be used in the methods disclosed herein, a recombinant expression vector encoding both the antibody heavy chain and the antibody light chain is introduced into SP2/0 cells by electroporation. In some embodiments of the expression systems, a recombinant expression vector encoding both the antibody heavy chain and the antibody light chain is introduced into CHO cells by standard techniques such as lipofection.

Within the recombinant expression vector, the antibody heavy and light chain genes are each operatively linked to murine or human Immunoglobulin heavy chain (IgH), CMV enhancer, metallothioneine or AdMLP promoter regulatory elements to drive high levels of transcription of the genes. The recombinant expression vector also carries a DHFR gene, which allows for selection of SP2/0 cells that have been transfected with the vector using methotrexate selection/amplification. Alternatively, the recombinant expression vector containing the antibody heavy and light chain genes operatively linked to murine or human IgH, CMV enhancer/AdMLP/metallothioncine promoter regulatory elements and a DHFR gene can be used to transfect SP2/0 or CHO cells that are dhfr-. SP2/0 or CHO cells transfected with the vector can be selected and the level of gene expression in the vector amplified by increasing the levels of methotrexate in the culture. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium.

### 7.4 Pharmaceutical Compositions

Provided herein are also pharmaceutical compositions comprising bispecific antibodies or antigen-binding fragments with one specificity against CD22, CD33 or CD74, and the other specificity against Aβ protein that can be used in methods disclosed herein. In some embodiments, the pharmaceutical composition comprises a therapeutically effective amount of the bispecific antibodies or antigen-binding fragments disclosed herein and a pharmaceutically acceptable carrier. In some embodiments, the pharmaceutical compositions are useful in the treatment of an Aβ-related disease or disorder or a neuroinflammation-related disease or disorder. In some embodiments, the pharmaceutical compositions are useful in treating AD. In some embodiments, the pharmaceutical compositions are useful in inhibiting AD progression in a subject (e.g., a human patient). In some embodiments, the pharmaceutical compositions are useful in ameliorating cognitive impairment in a subject (e.g., a human patient).

The amount of therapeutic antibody which can be combined with a carrier material in the pharmaceutical compositions disclosed herein can vary. In some embodiments, the amount of antibodies present in the pharmaceutical compositions is the amount that produces a therapeutic effect. Generally, out of one hundred percent, this amount will range from about 0.01 percent to about ninety-nine percent of active ingredient, from about 0.1 percent to about 70 percent, or from about 1 percent to about 30 percent of active ingredient in combination with a pharmaceutically acceptable carrier.

The pharmaceutical compositions provided herein comprise bispecific antibodies or antigen-binding fragments with one specificity against CD22, CD33 or CD74, and other specificity against Aβprotein provided herein, or related antibodies identified by the methods disclosed herein. The anti-CD22, anti-CD33, or anti-CD74 bispecific antibodies or antigen-binding fragments can be present at various concentrations. In some embodiments, the pharmaceutical compositions provided herein comprise soluble anti-CD22, anti-CD33, or anti-CD74 bispecific antibodies or antigen-binding fragments provided herein at 1-1000 mg/ml. In some embodiments, the pharmaceutical compositions comprise soluble anti-CD22, anti-CD33, or anti-CD74 bispecific antibodies or antigen-binding fragments provided herein at 10-500 mg/ml, 10-400 mg/ml, 10-300 mg/ml, 10-200 mg/ml, 10-100 mg/ml, 20-100 mg/ml, or 50-100 mg/ml. In some embodiments, the pharmaceutical compositions provided herein comprise anti-CD22, anti-CD33, or anti-CD74 bispecific antibodies or antigen-binding fragments provided herein at about 10 mg/ml, about 20 mg/ml, about 30 mg/ml, about 40 mg/ml, about 50 mg/ml, about 60 mg/ml, about 70 mg/ml, about 80 mg/ml, about 90 mg/ml, about 100 mg/ml, about 120 mg/ml, about 150 mg/ml, about 180 mg/ml, about 200 mg/ml, about 300 mg/ml, about 500 mg/ml, about 800 mg/ml, or about 1000 mg/ml. Dosages can be readily adjusted by those skilled in the art; for example, a decrease in purity requires an increase in dosage.

The pharmaceutical compositions provided herein can be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Examples of suitable aqueous and nonaqueous carriers that can be employed in the pharmaceutical compositions or formulations described herein include water, ethanol, polyols (such as glycerol, propylene glycol, polyethylene glycol, and the like), and suitable mixtures thereof, vegetable oils, such as olive oil, and injectable organic esters, such as ethyl olcate.

Proper fluidity can be maintained, for example, by the use of coating materials, such as lecithin, by the maintenance of the required particle size in the case of dispersions, and by the use of surfactants. In some embodiments, pharmaceutical compositions provided herein are in the form of injectable or infusible solutions. In some embodiments, the pharmaceutical composition is an aqueous formulation. Such a formulation is typically a solution or a suspension, but can also include colloids, dispersions, emulsions, and multi-phase materials. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water. The composition can be formulated as a solution, microemulsion, liposome, or other ordered structure suitable to high drug concentration.

In some embodiments, the pharmaceutical compositions disclosed herein are freeze-dried, to which the physician or the patient adds solvents and/or diluents prior to usc.

The pharmaceutical compositions provided herein can comprise a pharmaceutically acceptable carrier. Pharmaceutically acceptable carriers include any and all solvents, dispersion media, coatings, antibacterial and antifungal agents, isotonic and absorption delaying agents, and the like that are physiologically compatible. Examples include one or more of water, saline, phosphate buffered saline, dextrose, glycerol, ethanol and the like, as well as combinations thereof. In some embodiments, the pharmaceutical acceptable carriers include isotonic agents, for example, sugars, polyalcohols such as mannitol, sorbitol, or sodium chloride in the composition.

In some embodiments, the pharmaceutical acceptable carriers further comprise minor amounts of auxiliary substances such as wetting or emulsifying agents, preservatives or buffers, which enhance the shelf life or effectiveness of the antibody or antigen-binding fragment. In some embodiments, the carrier is suitable for intravenous, intramuscular, subcutaneous, parenteral, spinal or epidermal administration (e.g., by injection or infusion). Depending on the route of administration, the active ingredient (i.e., anti-CD22, anti-CD33, or anti-CD74 bispecific antibodies or antigen-binding fragments), can be coated in a material to protect the active ingredient from the action of acids and other natural conditions that can inactivate the active ingredient.

Provided herein are also kits for preparation of pharmaceutical compositions having the anti-CD22, anti-CD33, or anti-CD74 bispecific antibodies or antigen-binding fragments disclosed herein. In some embodiments, the kit comprises the anti-CD22, anti-CD33, or anti-CD74 bispecific antibodies or antigen-binding fragments disclosed herein and a pharmaceutically acceptable carrier in one or more containers. In another embodiment, the kits can comprise anti-CD22, anti-CD33, or anti-CD74 bispecific antibodies or antigen-binding fragments disclosed herein for administration to a subject. In specific embodiments, the kits comprise instructions regarding the preparation and/or administration of the anti-CD22, anti-CD33, or anti-CD74 bispecific antibodies or antigen-binding fragments.

In some embodiments, the pharmaceutical composition or formulation disclosed herein comprises: (a) anti-CD22, anti-CD33, or anti-CD74 bispecific antibodies or antigen-binding fragments disclosed herein; (b) a buffering agent; (c) a stabilizing agent; (d) a salt; (c) a bulking agent; and/or (f) a surfactant. In some embodiments, the pharmaceutical composition or formulation is stable for at least 1 month, at least 2 months, at least 3 months, at least 6 months, at least 1 year, at least 2 years, at least 3 years, at least 5 years or more. In some embodiments, the pharmaceutical composition or formulation is stable when stored at 4° C., 25° C., or 40° C. In some embodiments, provided herein are also pharmaceutical compositions or formulations that improve the stability of the anti-CD22, anti-CD33, or anti-CD74 bispecific antibodies or antigen-binding fragments to allow for their long-term storage. The pharmaceutical compositions disclosed herein can further comprise one or more of a preservative, a tonicity agent, a chelating agent, a stabilizer and/or a surfactant, as well as various combinations thereof. The use of preservatives, isotonic agents, chelating agents, stabilizers and surfactants in pharmaceutical compositions is well-known to the skilled person. Reference may be made to Remington: THE SCIENCE AND PRACTICE OF PHARMACY, 19th edition, 1995.

Buffering agents useful in the pharmaceutical compositions or formulations disclosed herein can be a weak acid or base used to maintain the acidity (pH) of a solution near a chosen value after the addition of another acid or base. Suitable buffering agents can maximize the stability of the pharmaceutical formulations by maintaining pH control of the formulation. Suitable buffering agents can also ensure physiological compatibility or optimize solubility. Rheology, viscosity and other properties can also depend on the pH of the formulation. Common buffering agents include, but are not limited to, histidine, citrate, succinate, acetate and phosphate. In some embodiments, a buffering agent comprises histidine (e.g., L-histidine) with isotonicity agents and potentially pH adjustment with an acid or a base known in the art. In certain embodiments, the buffering agent is L-histidine. In certain embodiments, the pH of the formulation is maintained between about 2 and about 10, or between about 4 and about 8.

Stabilizing agents are added to a pharmaceutical product to stabilize that product. Such agents can stabilize proteins in different ways. Common stabilizing agents include, but are not limited to, amino acids such as glycine, alanine, lysine, arginine, or threonine, carbohydrates such as glucose, sucrose, trehalose, rafftnose, or maltose, polyols such as glycerol, mannitol, sorbitol, cyclodextrins or destrans of any kind and molecular weight, or PEG. In some embodiments, the stabilizing agent is chosen to maximize the stability of FIX polypeptide in lyophilized preparations. In certain embodiments, the stabilizing agent is sucrose and/or arginine.

Bulking agents can be added to a pharmaceutical composition or formulation to add volume and mass to the product, thereby facilitating precise metering and handling thereof. Common bulking agents include, but are not limited to, lactose, sucrose, glucose, mannitol, sorbitol, calcium carbonate, or magnesium stearate.

Surfactants are amphipathic substances with lyophilic and lyophobic groups. A surfactant can be anionic, cationic, zwitterionic, or nonionic. Examples of nonionic surfactants include, but are not limited to, alkyl ethoxylate, nonylphenol ethoxylate, amine ethoxylate, polyethylene oxide, polypropylene oxide, fatty alcohols such as cetyl alcohol or oleyl alcohol, cocamide MEA, cocamide DEA, polysorbates, or dodecyl dimethylamine oxide. In some embodiments, the surfactant is polysorbate 20 or polysorbate 80.

Pharmaceutical compositions disclosed herein can also include a pharmaceutically acceptable antioxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

These compositions can also contain adjuvants such as preservatives, wetting agents, emulsifying agents and dispersing agents. Prevention of presence of microorganisms can be ensured both by sterilization procedures, supra, and by the inclusion of various antibacterial and antifungal agents, for example, paraben, chlorobutanol, phenol sorbic acid, and the like. It can also be desirable to include isotonic agents, such as sugars, sodium chloride, and the like into the compositions. In addition, prolonged absorption of the injectable pharmaceutical form can be brought about by the inclusion of agents which delay absorption such as aluminum monostearate and gelatin.

Pharmaceutical compositions or formulations typically must be sterile and stable under the conditions of manufacture and storage. Pharmaceutically acceptable carriers include sterile aqueous solutions or dispersions and sterile powders for the extemporaneous preparation of sterile injectable solutions or dispersion. Sterile injectable solutions can be prepared by incorporating the therapeutic antibody or antigen-binding fragment in the required amount in an appropriate solvent with one or a combination of ingredients enumerated above, as required, followed by filtered sterilization. The use of such media and agents for pharmaceutically active substances is known in the art. Generally, dispersions are prepared by incorporating the active compound into a sterile vehicle that contains a basic dispersion medium and the required other ingredients from those enumerated above. In the case of sterile powders for the preparation of sterile injectable solutions, some methods of preparation are vacuum drying and freeze-drying (lyophilization) that yield a powder of the active ingredient plus any additional desired ingredient from a previously sterile-filtered solution thereof.

The pharmaceutical compositions disclosed herein can be prepared with carriers that protect the active ingredient against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and poly lactic acid. Many methods for the preparation of such formulations are patented or generally known to those skilled in the art. Sec. e.g., SUSTAINED AND CONTROLLED RELEASE DRUG DELIVERY SYSTEMS, J. R. Robinson, ed., Marcel Dekker, Inc., New York, 1978.

In some embodiments, the anti-CD22, anti-CD33, or anti-CD74 bispecific antibodies or antigen-binding fragments described herein can be formulated to ensure proper distribution in vivo. For example, the BBB excludes many highly hydrophilic compounds. To facilitate the therapeutic antibodies described herein to cross the BBB, they can be formulated, for example, in liposomes. For methods of manufacturing liposomes, see, e.g., U.S. Pat. Nos. 4,522,811; 5,374,548; and 5,399,331. The liposomes can comprise one or more moieties which are selectively transported into specific cells or organs, thus enhance targeted drug delivery (see, e.g., V. V. Ranade (1989) J. Clin. Pharmacol. 29:685). Exemplary targeting moieties include folate or biotin (see, e.g., U.S. Pat. No. 5,416,016 to Low et al) mannosides (Umczawa et al., (1988) Biochem. Biophys. Res. Commun. 153:1038); antibodies (P. G. Bloeman et al. (1995) FEBS Lett. 357:140; M. Owais et al. (1995) Antimicrob. Agents Chemother. 39: 180); surfactant protein A receptor (Briscoe et al. (1995) Am. J. Physiol. 1233:134); p120 (Schreier et al. (1994) J. Biol. Chem. 269:9090); see also K. Keinanen; M. L. Laukkanen (1994) FEBS Lett. 346:123; J. J. Killion; I. J. Fidler (1994) Immunomethods 4:273.

### 7.5 Methods of Treatment

As described in sections above, provided herein are medical uses of bispecific antibodies and antigen-binding fragments with one specificity against CD22, CD33 or CD74, and the other specificity against Aβ protein in treating Aβ or neuroinflammation-related diseases or disorders. Any anti-CD22, anti-CD33 or anti-CD74 bispecific antibody or antigen-binding fragment disclosed herein or identified using the screening methods disclosed herein can be used in the methods disclosed herein. In some embodiments, provided herein are methods of promoting removal of Aβ plaque in a subject in need thereof. In some embodiments, provided herein are also methods of treating an Aβ-related disease or disorder in a subject in need thereof. In some embodiments, provided herein are also methods of reducing neuroinflammation in a subject in need thereof. In some embodiments, provided herein are also methods of treating a disease or disorder associated with neuroinflammation in a subject in need thereof. In some embodiments, the methods provided herein prevent synaptic phagocytosis and neuronal death. In some embodiments, the methods provided herein prevent synaptic phagocytosis and neuronal death by at least 20%, at least 30, at least 40%, at least over 50%, or at least over 60%. In some embodiments, the subject is a human. In some embodiments, the bispecific antibody or antigen-binding fragment thereof specifically binds to human CD22, human CD33 or CD74.

The methods of promoting removal of Aβ plaque, reducing neuroinflammation, treating an Aβ-related disease or disorder, and/or treating a disease or disorder disclosed herein comprise administering to the subject a therapeutically effective amount of a bispecific antibody or antigen-binding fragment thereof that specifically binds to CD22, CD33 or CD74 on one end, and specifically binds to Aβ on the other end, wherein the bispecific antibody or antigen-binding fragment (a) promotes removal of Aβ via the induction of surface receptor/antigen, namely, CD22, CD33 or CD74, internalization and/or (b) reducing neuroinflammation by microglia cells, or any combination thereof.

In some embodiments, the Aβ-related disease or disorder and/or neuroinflammation-related disease or disorder can be clinical or pre-clinical AD, prodromal AD, Down's syndrome, clinical or pre-clinical amyloid angiopathy (CAA), Parkinson's disease, multi-infarct dementia, cerebral amyloid angiopathy, glaucoma, pre-eclampsia, cognitive impairment, memory loss, or a vascular disorder caused by pathogenic Aβ peptide in blood vessel.

Subjects suitable for the present methods include human patients in whom the removal of Aβplaque and/or reduction in neuroinflammation would be desirable. In some embodiments, the subjects to be treated with the methods disclosed herein are diagnosed with an Aβ-related or neuroinflammation-related disease or disorder, which can be clinical or pre-clinical AD, prodromal AD, Down's syndrome, clinical or pre-clinical amyloid angiopathy (CAA), Parkinson's disease, multi-infarct dementia, cerebral amyloid angiopathy, glaucoma, pre-eclampsia, cognitive impairment, memory loss, or a vascular disorder caused by pathogenic Aβ peptide in blood vessel. In some embodiments, the subjects to be treated with the methods disclosed herein are at risk of developing an Aβ-related or neuroinflammation-related disease or disorder, which can be clinical or pre-clinical AD, prodromal AD, Down's syndrome, clinical or pre-clinical amyloid angiopathy (CAA), Parkinson's disease, multi-infarct dementia, cerebral amyloid angiopathy, glaucoma, pre-eclampsia, cognitive impairment, memory loss, or a vascular disorder caused by pathogenic Aβ peptide in blood vessel. The subject can be a mammal. In some embodiments, the subject is a human.

In some embodiments, the subject is diagnosed with AD. In some embodiments, the subject is at risk of developing AD. In some embodiments, the subject has pre-clinical AD. In some embodiments, the subject has clinical AD. In some embodiments, the subject has prodromal AD. In some embodiments, the subject to be treated with the methods disclosed herein have been with the standard therapy for AD. In some embodiments, the subject has not been previously treated.

The bispecific antibodies or antigen-binding fragments with one specificity against CD22, CD33 or CD74, and other specificity against Aβ or pharmaceutical compositions provided herein can be administered to a subject by any methods known in the art, including, but not limited to, intravenous administration, subcutaneous administration, intramuscular administration, intracranial administration, intrathecal administration, intraventricular administration, intraperitoneal administration, spinal administration, intranasal administration, intrapleural administration, topical administration, or intradermal administration.

In some embodiments, the bispecific antibodies or antigen-binding fragments with one specificity against CD22, CD33 or CD74, and other specificity against Aβ or pharmaceutical compositions provided herein can be administered to a subject using parenteral administration. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrastemal injection and infusion. In some embodiments, the anti-CD22, anti-CD33 or anti-CD74 bispecific antibodies or antigen-binding fragments are administered by intravenous infusion or injection. In some embodiments, anti-CD22, anti-CD33, or anti-CD74 bispecific antibodies or antigen-binding fragments are administered by intramuscular injection. In some embodiments, anti-CD22, anti-CD33, or anti-CD74 bispecific antibodies or antigen-binding fragments are administered by subcutaneous injection.

Blood brain barrier tightly regulates the substance transport in and out of the brain. As known in the art, patients with AD are associated with vascular leakage and the IgG penetration into the brain could reach approximately 0.2%. As such, therapeutical antibodies delivered systematically can cross BBB and reach the lesion site. Alternatively, in some embodiments, the antibodies or antigen-binding fragments provided herein can be delivered locally using intracranial administration. In some embodiments, intraventricular administration is adopted.

Bispecific antibodies or antigen-binding fragments with one specificity against CD22, CD33 or CD74, and other specificity against Aβ or pharmaceutical compositions provided herein can be administered with medical devices known in the art. For example, in some embodiments, a needleless hypodermic injection device can be used, such as the devices disclosed in U.S. Pat. Nos. 5,399,163; 5,383,851; 5,312,335; 5,064,413; 4,941,880; 4,790,824; or 4,596,556. Examples of well-known implants and modules for use described herein include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

In some embodiments, bispecific antibodies or antigen-binding fragments with one specificity against CD22, CD33 or CD74, and other specificity against Aβ disclosed herein can be orally administered, for example, with an inert diluent or an assimilable edible carrier. The therapeutic antibodies also be enclosed in a hard or soft shell gelatin capsule, compressed into tablets, or incorporated directly into the subject's diet. For oral therapeutic administration, the therapeutic antibodies can be incorporated with excipients and used in the form of ingestible tablets, buccal tablets, troches, capsules, elixirs, suspensions, syrups, wafers, and the like.

The methods provided herein comprise administering a therapeutically effective amount of the bispecific antibodies or antigen-binding fragments with one specificity against CD22, CD33 or CD74, and other specificity against Aβ described herein. Actual dosage levels of the therapeutic antibodies can be varied so as to obtain an amount which is effective to achieve the desired therapeutic response for a particular patient, without being toxic to the patient. The selected dosage level will depend upon a variety of pharmacokinetic factors including the activity of the particular compositions described herein, the route of administration, the time of administration, the rate of excretion, the duration of the treatment, other drugs, compounds and/or materials used in combination with the particular compositions employed, the age, sex, weight, condition, general health and prior medical history of the patient being treated, and like factors well known in the medical arts.

Generally, the dosage can range from, e.g., about 0.1 to 100 mg/kg of the host body weight for a single dose. In some embodiments, the bispecific antibodies or antigen-binding fragments with one specificity against CD22, CD33 or CD74, and other specificity against Aβ is administered at about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 20 mg/kg, about 30 mg/kg, about 40 mg/kg, about 50 mg/kg, about 60 mg/kg, about 70 mg/kg, about 80 mg/kg, about 90 mg/kg per. In some embodiments, the anti-CD22, anti-CD33 or anti-CD74 bispecific antibody or antigen-binding fragment is administered at about 1 mg/kg. In some embodiments, the anti-CD22, anti-CD33, or anti-CD74 bispecific antibody or antigen-binding fragment is administered at about 5 mg/kg. In some embodiments, the anti-CD22, anti-CD33, or anti-CD74 bispecific antibody or antigen-binding fragment is administered at about 10 mg/kg. In some embodiments, the anti-CD22, anti-CD33, or anti-CD74 bispecific antibody or antigen-binding fragment is administered at about 20 mg/kg. In some embodiments, the anti-CD22, anti-CD33, or anti-CD74 bispecific antibody or antigen-binding fragment is administered at about 40 mg/kg. In some embodiments, the anti-CD22, anti-CD33, or anti-CD74 bispecific antibody or antigen-binding fragment is administered at about 60 mg/kg. In some embodiments, the anti-CD22, anti-CD33, or anti-CD74 bispecific antibody or antigen-binding fragment is administered at about 100 mg/kg.

In some embodiments, the bispecific antibodies or antigen-binding fragments with one specificity against CD22, CD33 or CD74, and other specificity against Aβ is administered at a dose within a range of about 1 to 5 mg/kg, about 1 to 10 mg/kg, about 1 to 20 mg/kg, about 1 to 50 mg/kg, about 1 to 100 mg/kg, about 5 to 10 mg/kg, about 5 to 20 mg/kg, about 5 to 50 mg/kg, about 5 to 100 mg/kg, about 10 to 50 mg/kg, or about 10 to 100 mg/kg. In some embodiments, the anti-CD22, anti-CD33, or anti-CD74 bispecific antibody or antigen-binding fragment is administered at a dose within a range of about 1 to 5 mg/kg. In some embodiments, the anti-CD22, anti-CD33, or anti-CD74 bispecific antibody or antigen-binding fragment is administered at a dose within a range of about 1 to 10 mg/kg. In some embodiments, the anti-CD22, anti-CD33, or anti-CD74 bispecific antibody or antigen-binding fragment is administered at a dose within a range of about 1 to 50 mg/kg. In some embodiments, the anti-CD22, anti-CD33, or anti-CD74 bispecific antibody or antigen-binding fragment is administered at a dose within a range of about 10 to 50 mg/kg. In some embodiments, the anti-CD22, anti-CD33, or anti-CD74 bispecific antibody or antigen-binding fragment is administered at a dose within a range of about 10 to 100 mg/kg.

In some embodiments, methods provided herein comprise administering the bispecific antibodies or antigen-binding fragments with one specificity against CD22, CD33 or CD74, and other specificity against Aβ at a dose of about 10-2000 mg. In some embodiments, the dose is about 10 mg, about 50 mg, about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg about 1600 mg, about 1700 mg, about 1800 mg, about 1900 mg, or about 2000 mg. In some embodiments, the antibody is administered at a dose of 100 mg. In some embodiments, the antibody is administered at a dose of 300 mg. In some embodiments, the antibody is administered at a dose of 600 mg. In some embodiments, the antibody is administered at a dose of 900 mg. In some embodiments, the antibody is administered at a dose of 1200 mg.

In some embodiments, methods provided herein comprise administering the bispecific antibodies or antigen-binding fragments with one specificity against CD22, CD33 or CD74, and other specificity against Aβ at a dose within a range of about 10-50 mg, 10-100 mg, 10-200 mg, 100-300 mg, 100-500 mg, 300-600 mg, 300-900 mg, 300-1200 mg, 600-1200 mg, 600-1800 mg, or 1000-2000 mg. In some embodiments, the antibody is administered at a dose within the range of 100-500 mg. In some embodiments, the antibody is administered at a dose within the range of 300-600 mg. In some embodiments, the antibody is administered at a dose within the range of 300-900 mg. In some embodiments, the antibody is administered at a dose within the range of 600-1200 mg.

During treatment, it is common to start with a lower dose, which is later ramped up to a target dose. For illustrative purpose, in some embodiments, the methods provided herein comprise administering an anti-CD22, anti-CD33 or anti-CD74 bispecific antibody or antigen-binding fragment at a dose of about 100 mg, which is gradually ramped up to a target dose of about 600 mg.

Subjects can be administered at such doses daily, on alternative days, weekly, biweekly, monthly, or according to any other schedule determined by empirical analysis. An exemplary treatment entails administration in multiple dosages over a prolonged period, for example, of at least six months. In some embodiments, the methods provided herein comprise weekly administering an anti-CD22, anti-CD33 or anti-CD74 bispecific antibody or antigen-binding fragment. In some embodiments, the methods comprise biweekly administration. In some embodiments, the methods comprise monthly administration. In some embodiments, the bispecific antibodies or antigen-binding fragments with one specificity against CD22, CD33 or CD74, and other specificity against Aβ is subcutaneously administered weekly, biweekly, or monthly. In some embodiments, the bispecific antibodies or antigen-binding fragments with one specificity against CD22, CD33 or CD74, and other specificity against Aβ is intravenously administered weekly, biweekly, or monthly.

The bispecific antibodies or antigen-binding fragments with one specificity against CD22, CD33 or CD74, and other specificity against Aβ can be administered up to 3 months, 6 months, 9 months, 12 months, 18 months, 24 months, 30 months, or 36 months, as necessary and appropriate. In some embodiments, the treatment lasts at least 3 months. In some embodiments, the treatment lasts at least 6 months. In some embodiments, the treatment lasts at least 12 months. In some embodiments, the treatment lasts at least 24 months.

All permutations and combinations of the various embodiments of, e.g., round of administration, dosage, treatment frequency, and length of treatment are expressly contemplated herein and can be adopted in the therapeutic methods disclosed herein.

For illustrative purposes, the following treatment regimen can be adopted in the methods disclosed herein that comprise administering of bispecific antibodies or antigen-binding fragments with one specificity against CD22, CD33 or CD74, and other specificity against Aβ that is either disclosed herein or identified in methods disclosed herein:

In some embodiments, the therapeutic antibody is administered intravenously or subcutaneously at a dose of about 10 mg/kg every 4 weeks and at least 21 days apart. In some embodiments, the following titration schedule is included: Infusions 1-2:1 mg/kg IV; Infusions 3-4:3 mg/kg IV; Infusions 5-6:6 mg/kg IV; Infusion 7 and beyond: 10 mg/kg IV.

In some embodiments, the therapeutic antibody is administered intravenously or subcutaneously at a single dose of 10, 20, or 40 mg/kg, the second of 10 mg/kg every other week for 24 weeks, and the third of 10 or 20 mg/kg every month for 16 months.

In some embodiments, the therapeutic antibody is administered intravenously or subcutaneously at a dose of about 250 mg weekly, or 500 mg biweekly for up to 2 years. In some embodiments, the treatment starts with monthly shots of about 120 mg.

Dosage regimens can be adjusted to provide the optimum desired response (e.g., a therapeutic or prophylactic response). For example, a single bolus can be administered, several divided doses can be administered over time, or the dose can be proportionally reduced or increased as indicated by the exigencies of the therapeutic situation. It is especially advantageous to formulate parenteral compositions in dosage unit form for case of administration and uniformity of dosage. Dosage unit form as used herein refers to physically discrete units suited as unitary dosages for the mammalian subjects to be treated; each unit containing a predetermined quantity of therapeutic antibody calculated to produce the desired therapeutic effect in association with the required pharmaceutical carrier. It is to be noted that proper dosing varies with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

In the treatment of an Aβ-related disease or disorder or neuroinflammation related disease or disorder, sometimes the disease or disorder can be cured with methods provided herein, but any clinical improvement constitutes a benefit. In some embodiments, the methods provided herein reduce amyloid by an average of about 50 centiloids (CL), about 60 CL, about 70 CL, about 80 CL, about 90 CL, about 95 CL, or about 99 CL. In some embodiments, the methods provided herein reduce amyloid by an average of about 50 CL. In some embodiments, the methods provided herein reduce amyloid by an average of about 80 CL. In some embodiments, the methods provided herein reduce amyloid by an average of about 90 CL. In some embodiments, the methods provided herein reduce amyloid by an average of 50 to 100 CL, 60 to 100 CL, 70 to 100 CL, 80 to 100 CL, or 90 to 100 CL. In some embodiments, methods provided herein reduce amyloid by an average of 50 to 100 CL. In some embodiments, methods provided herein reduce amyloid by an average of 90 to 100 CL. In some embodiments, the methods provided herein reduce neuroinflammation. In some embodiments, methods provided herein reduce vasogenic edema. In some embodiments, methods provided herein prevent synaptic phagocytosis and neuronal death. In some embodiments, methods provided herein prevent the onset of AD, or delay or halt the progression AD. In some embodiments, methods provided herein ameliorate the symptoms of AD. In some embodiments, methods provided herein ameliorate cognitive impairment. In some embodiments, methods provided herein delay the onset of cognitive impairment.

Compared to therapeutic antibodies that cross-link FcγR, the bispecific antibodies or antigen-binding fragments with one specificity against CD22, CD33 or CD74, and other specificity against Aβdisclosed herein have no or reduced vascular side effects (i.e., ARIA-E, ARIA-H). Additionally, in some embodiments, methods provided herein reduce vasogenic edema.

The bispecific antibodies or antigen-binding fragments with one specificity against CD22, CD33 or CD74, and other specificity against Aβ disclosed herein can be administered by a variety of methods known in the art. As appreciated by those skilled in the art, the route and/or mode of administration varies depending upon the desired results. In some embodiments, the therapeutic antibody can be prepared with a carrier that protects it against rapid release, such as a controlled release formulation, including implants, transdermal patches, and microencapsulated delivery systems. Biodegradable, biocompatible polymers can be used, such as ethylene vinyl acetate, polyethylene glycol (PEG), polyanhydrides, polyglycolic acid, collagen, polyorthoesteers, and polylactic acid. Many methods for the preparation of such formulations are patented or generally known to those skilled in the art. Sec, e.g., SUSTAINED AND CONTROLLED RELEASE DRUG DELIVERY SYSTEMS, J. R. Robinson ed., Marcel Dekker, Inc., New York, 1978. In therapeutic applications, a relatively high dosage at relatively short intervals is sometimes required until progression of the disease is reduced or terminated, and until the patient shows partial or complete amelioration of symptoms of disease.

Combination therapy using agents with different mechanisms of action can result in additive or synergetic effects. Combination therapy can allow for a lower dose of each agent than is used in monotherapy, thereby reducing toxic side effects and/or increasing the therapeutic index of the agent disclosed herein. Combination therapy can decrease the likelihood that drug-resistance would develop. In some embodiments, the additional therapy results in an increase in the therapeutic index of the antibodies or antigen-binding fragments, or pharmaceutical compositions described herein. In some embodiments, the additional therapy results in a decrease in the toxicity and/or side effects of the antibodies or antigen-binding fragments or pharmaceutical compositions described herein. In some embodiments, the anti-CD22, anti-CD33 or anti-CD74 bispecific antibodies or antigen-binding fragments, or pharmaceutical compositions described herein can be administered in combination with an additional therapy.

In some embodiments, the second therapeutic agent is a Tau aggregation inhibitor, a Tau protein modulator, a cholinesterase inhibitor, an acetylcholinesterase inhibitor, an N-methyl D-aspartate (NMDA) antagonist, a β-secretase inhibitor, or an insulin sensitizer. In some embodiments, the second therapeutic agent can be a second antibody that suppresses the release of pro-inflammatory cytokines, or an agent that enhances microglia cell phagocytic activities.

In some embodiments, the second therapeutic agent is selected from the group consisting of AL003 (AbbVie), gemtuzumab, lintuzumab, ozogamicin, vadastuximab talirine, and BI836858. In some embodiments, the second therapeutic agent is AL003. In some embodiments, the second therapeutic agent is gemtuzumab. In some embodiments, the second therapeutic agent is lintuzumab.

In some embodiments, the second therapeutic agent is a Tau aggregation inhibitor. In some embodiments, the second therapeutic agent is methylene blue derivative LMTX (also known as LMTM or TRx0237), or curcumin.

In some embodiments, the second therapeutic agent is a Tau protein modulator. In some embodiments, the second therapeutic agent is memantine, sodium selenate, alvocidib, seliciclib, Tideglusib, lithium, salsalate, or MK-8719.

In some embodiments, the second therapeutic agent is a cholinesterase inhibitor or an acetylcholinesterase inhibitor. In some embodiments, the second therapeutic agent is donepezil, rivastigmine, or galantamine.

In some embodiments, the second therapeutic agent is NMDA antagonist. In some embodiments, the second therapeutic agent is memantine.

The second therapeutic agent can be administered prior to, concurrently with, or subsequent to administration of the anti-CD22, ant-CD33 or anti-CD74 bispecific antibodies or antigen-binding fragments or pharmaceutical compositions described herein. Combined administration can include co-administration, either in a single pharmaceutical formulation or using separate formulations, or consecutive administration in either order but generally within a time period such that all active agents can exert their biological activities simultaneously. A person skilled in the art can readily determine appropriate regimens for administering a pharmaceutical composition described herein and an additional therapy in combination, including the timing and dosing of an additional agent to be used in a combination therapy, based on the needs of the subject being treated.

All papers, publications and patents cited in this specification are herein incorporated by reference as if each individual paper, publication or patent were specifically and individually indicated to be incorporated by reference and are incorporated herein by reference to disclose and describe the methods and/or materials in connection with which the publications are cited. However, mention of any reference, article, publication, patent, patent publication, and patent application cited herein is not, and should not be taken as an acknowledgment or any form of suggestion that they constitute valid prior art or form part of the common general knowledge in any country in the world.

Unless the context indicates otherwise, it is specifically intended that the various features described herein can be used in any combination.

Unless otherwise defined, all technical and scientific terms used herein have the same meaning as commonly understood by one of ordinary skill in the art to which this disclosure belongs.

### 7.6 Experimental

### 7.6.1 Example 1: CD22, CD33 and CD74 Expression in Human Microglia Cell Line

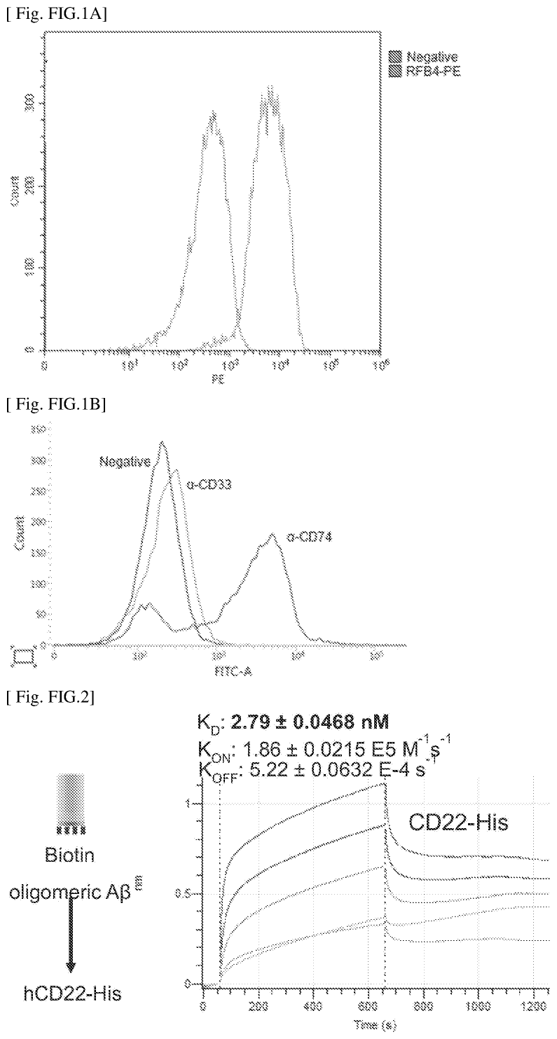

The expression levels of CD22, CD33 and CD74 on human microglia cells are examined by flow cytometry using human microglia cell line, HMC-3. Briefly, HMC-3 cells are seeded onto 6 well plate at a density of 0.03million cells/cm². Cells are detached with TrypLE™ Express enzyme and centrifuged at 300×g for 5 minutes. Cells are washed once with PBS (pH 7.4) and incubated either with anti-CD22 antibody, anti-CD33 antibody or anti-CD74 antibody for 1 hour on ice. Cell pellets are washed twice and PBS and stained with anti-human Fc antibody conjugated with AlexFluor 488 for 1 hour on ice. Cells are analyzed with flow cytometry following standard procedures known to those skilled in the art. FIGS. 1A and 1B demonstrate the typical flow cytometry graphs on the expression of CD22 (FIG. 1A), and CD33 and CD74 (FIG. 1B) on HMC-3. The comparative levels of CD22, CD33 and CD74 expression in HMC-3 cells are summarized in Table I.

TABLE I

| | HMC-3 |
|---|---|
| CD22 | ++ |
| CD33 | +++ |
| CD74 | +++ |

### 7.6.2 Example 2: Binding of CD22 to Oligomeric Aβ1-42 In Vitro

Octet 96e SPR system was used to analyze binding of CD22 to Aβ1-42 in vitro. Streptavidin Biosensor was loaded with 10 μg/mL biotinylated oligomeric Aβ1-42; and the interaction with CD22 of various concentration was detected with the biosensor. The steps were as follows: first, streptavidin biosensor was loaded into 10 μg/mL biotinylated oligomeric Aβ1-42 for 5 minutes; second, biosensor was washed with kinetic buffer (PBS+0.02% Tween20, 0.1% BSA) for 10 seconds; third, Biosensor was loaded into various concentration of extracellular domain of CD22 (Sino Biological) for 5 minutes for association; fourth, Biosensor was loaded into kinetic buffer fro dissociation for 5 mins. As shown in the FIG. 2, CD22 binds to oligomeric Aβ1-42 at a K_{D} of about 2.79 nM.

### 7.6.3 Example 3: Generation and Characterization of Anti-CD22, CD33, CD74 Bispecific Antibody

As shown in previous inventions, we showed that CD22 binds Aβ1-42 oligomer with strong binding affinity. SM03 treatment enhances phagocytosis FITC-conjugated AB1-42 by HMC-3. SM03 also modulate neuroinflammation by suppressing HMC-3 activation.

To further improve the therapeutic strategy of Alzheimer disease, we have designed bispecific antibody that on one end binds to neuromodulating &internalizing antigen and on the other end binds to Aβ. We have chosen antibodies which binds to CD22, CD33 and CD74 as they are implicated in neuromodulation and are rapidly internalizing upon binding to their respective antigens. The different anti-CD22, CD33, CD74 and Aβ antibody that are used for the current invention is listed in Table II. Table III lists out the different combinations of anti-CD22, anti-CD33 and anti-CD74 binding moieties that are fused to different anti-Aβ antibodies in the final bispecific antibody constructs.

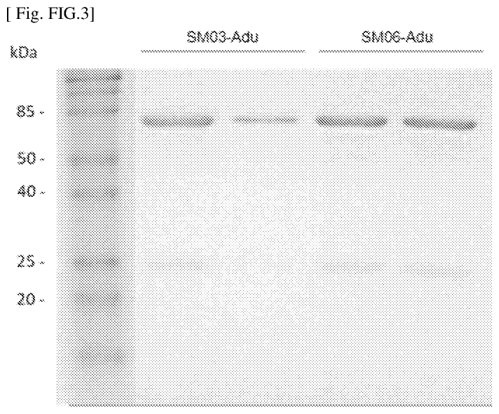

Bispecific antibodies are in the format of symmetrical design. Heavy chain of internalizing antigen was linked to Aβ-binding single chain Fv via a (G,S) x3 linker. Heavy chain-scFv and light chain were constructed in separated expression plasmid. Bispecific products were expressed by co-transfecting plasmid into Expi-CHO cells according to manufacturer's instructions. Bispecific antibody was analyzed with SDS-PAGE to examine its molecular size and ELISA to validate its antigen binding. All expressed bispecific antibodies regardless of their binding combinations exhibit a band of ˜80 kD characteristic of a heavy chain-scFv fusion protein, and a band size of ˜ 25 kD typical that of a light chain, when analyzed under reducing SDS-PAGE. An example of the reducing SDS-PAGE showing these two characteristic bands for SM03-Adu and SM06-Adu is shown in FIG. 3.

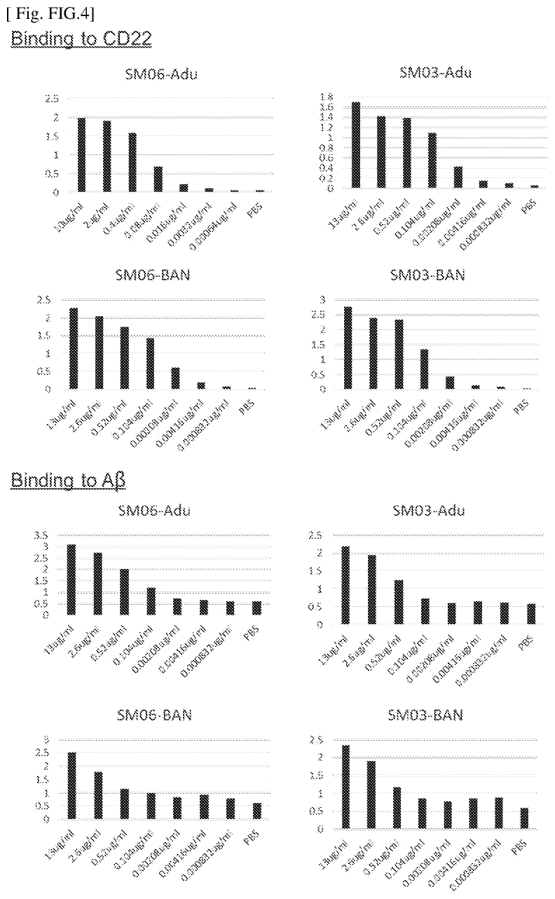

The binding of the different bispecific antibodies to their respective antigens is tested with standard ELISA method known to those skilled in the art. Using SM03-Adu as an example, 2 μg/mL of human CD22 protein (Sino Biological) or 5 μg/mL of Aβ oligomer are coated separately onto ELISA stripes (Santa Cruz) overnight at 4° C. or 1 hour at room temperature, respectively. ELISA stripes are blocked with 1% BSA in PBS. Stripes are washed 2 times with PBS. Bispecific antibodies are added in the following concentration, e.g. 10 μg/mL, 2 μg/mL, 0.4 μg/mL, 0.08 μg/mL, 0.016 μg/mL, 0.0032 μg/mL, 0.00064 μg/mL and incubated for 1 hour at room temperature. Stripes are washed 3 times with PBS. Stripes are incubated with anti-human Fc HRP-conjugated antibody diluted 1:6000 in PBS for 1 hour at room temperature. Fifty μL TMB (Invitrogem) is then added. After 30 minutes incubation at room temperature, 50 μL stop solution (2N H2SO4) is added to stop the reaction. Coloration are read by plate reader. As shown in FIG. 4, SM03-Adu, SM06-Adu, SM03-BAN, SM06-BAN bind to both CD22 and Aβ, confirming the binding specificities of the designed bispecific antibodies. Other bispecific antibodies are examined using the same protocol and the results are summarized in Table IV below.

TABLE II

| | Antibody |
|---|---|
| CD22 | SM03, SM06, hLL2 |
| CD33 | Gemtuzumab (Gem), HuMy9-6 (96), Lintuzumab (Lin) |
| CD74 | hLL1 |
| Aβ | Aducanumab (Adu), BAN2401 (BAN), Gantenerumab (Gan), |
| | Crenezumab (Cre), AD38 |

TABLE III

| Bispecific Antibody | Anti-surface Antigen Antibody | Aβ antibody |
|---|---|---|
| SM03-Adu | SM03 | Aducanumab |
| SM03-BAN | SM03 | BAN2401 |
| SM03-Gan | SM03 | Gantenerumab |
| SM03-Cre | SM03 | Crenezumab |
| SM06-Adu | SM06 | Aducanumab |
| SM06-BAN | SM06 | BAN2401 |
| SM06-Gan | SM06 | Gantenerumab |
| SM06-Cre | SM06 | Crenezumab |
| SM06-AD38 | SM06 | AD-38 |
| hLL2-Adu | hLL2 | Aducanumab |
| hLL2-Cre | hLL2 | Crenezumab |
| hLL2-Gan | hLL2 | Gantenerumab |
| hLL2-BAN | hLL2 | BAN2401 |
| Gem-Adu | Gemtuzumab | Aducanumab |
| 96-Adu | HuMy9-6 | Aducanumab |
| Lin-Adu | Lintuzumab | Aducanumab |
| hLL1-Adu | hLL1 | Aducanumab |

TABLE IV

| | Bispecific | Binding to | Binding to |
|---|---|---|---|
| Antigen | Antibody | internalizing antigen | oligomeric Aβ |
| CD22 | SM03-Adu | ++++ | ++++ |
| | SM03-BAN | ++++ | ++++ |
| | SM03-Gan | ++++ | ++++ |
| | SM03-Cre | ++++ | +++++ |
| | SM06-Adu | ++++ | ++++ |
| | SM06-BAN | ++++ | ++++ |
| | SM06-Gan | ++++ | ++++ |
| | SM06-Cre | ++++ | +++++ |
| | SM06-AD38 | ++++ | +++ |
| | hLL2-Adu | +++ | ++++ |
| | hLL2-Cre | +++ | +++++ |
| | hLL2-Gan | +++ | ++++ |
| | hLL2-BAN | +++ | ++++ |
| CD33 | Gem-Adu | +++++ | ++++ |
| | 96-Adu | ++++ | ++++ |
| | Lin-Adu | ++++ | ++++ |
| CD74 | hLL1-Adu | +++++ | ++++ |

Note:
“+++++” indicates highest binding while “+” indicates the weakest binding

### 7.6.4 Example 4: Binding of Bispecific Antibody to HMC-3

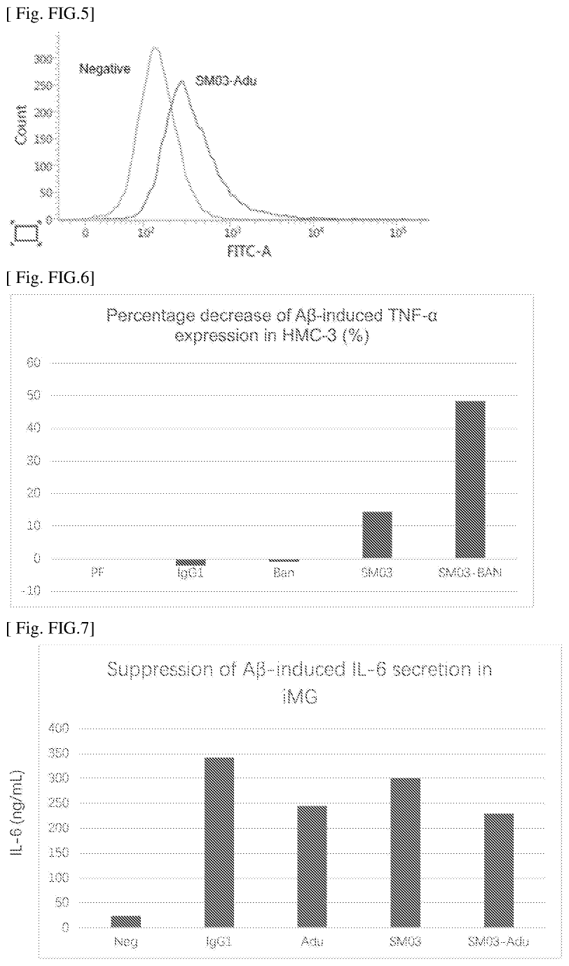

The binding of different bispecific antibodies to HMC-3 cells is evaluated. Briefly, HMC-3 cells are seeded onto 6 well plate at a density of 0.52 million cells/cm². After plating and culture for overnight, cells are detached with TrypLE™ Express enzyme. Cells are centrifuged at 300 g for 5 minutes. Cell pellets are washed once with phosphate buffer saline (pH7.4). Cells are fixed with 4% PFA in PBS for 5 minutes at room temperature. Cells are washed once with PBS. Bispecifc antibodies at a concentration of10 μg/mL are incubated with HMC-3 for 1 hour at room temperature. Cells are then washed twice with PBS. Cells are incubated with anti-human Fc Alexafluor 488-conjugated antibody diluted 1:1000 in PBS for 1 hour at room temperature. Cells are washed twice with PBS. Cell are centrifuged at 300 g for 5 minutes. Cell pellet is resuspended with 1% BSA in PBS and proceed to flow cytometry. FIG. 5 illustrates a representative result of HMC-3 cell binding by SM03-Adu. Other bispecific antibodies are examined using the above protocol and their results are summarized in Table V.

TABLE V

| Antigen | Bispecific Antibody | Expression level |
|---|---|---|
| CD22 | SM03-Adu | +++ |
| | SM03-BAN | +++ |
| | SM03-Gan | +++ |
| | SM03-Cre | +++ |
| | SM06-Adu | +++ |
| | SM06-BAN | +++ |
| | SM06-Gan | +++ |
| | SM06-Cre | +++ |
| | SM06-AD38 | +++ |
| | hLL2-Adu | +++ |
| | hLL2-Cre | +++ |
| | hLL2-Gan | +++ |
| | hLL2-BAN | +++ |
| CD33 | Gem-Adu | ++ |
| | 96-Adu | ++ |
| | Lin-Adu | ++ |
| CD74 | hLL1-Adu | ++++ |

Note:
“+++++” indicates highest binding while “+” indicates the weakest binding

### 7.6.5 Example 5: Effect of CD22-AB Bispecific Antibodies on Aβ-Induced TNF-α Expression in Human Microglia Cell Line

Briefly, HMC-3 cells are seeded at a density of 0.52 million cells/cm². After plating, cells are incubated with 5 μg/mL Aβ and cells are co-treated with antibodies. After 4 hours incubation, cells are lysed, and RNA are collected with RNAzol. Purified RNA is reversed transcribed to complementary DNA. The mRNA expression of TNF-α are examined by quantitative PCR. As shown in FIG. 6, SM03-BAN shows enhanced inhibition of TNF-α expression when compared with anti-Aβ antibody (BAN 2401) and anti-CD22 antibody (SM03).

### 7.6.6 Example 6: Effects of CD22-AB Bispecific Antibody on Aβ-Induced IL-6 Secretion in Human Induced Microglia Cell

iMG are generated by seeding 1.25 million human peripheral blood mononuclear cells onto 0.02% Geltrex-coated well in 24 well plate. Twenty-four hours after plating, the suspension cells are removed and the adhere monocyte are washed with PBS once. The adhered monocytes are differentiated with 0.1 μg/ml IL-34 and 0.01 ng/ml GM-CSF for 14 days. Fresh differentiation medium are change every 2-3 days. For the treatment, iMG are incubated with 5 μg/mL Aβ for 24 hours. iMGare co-treated with antibody. After 24 hour incubation, the supernatant are collect and Il-6 level are examined with IL-6 ELISA kit (R&D systems). FIG. 7 illustrates an enhanced suppressive effect of SM03-Adu on IL-6 release when compared to anti Aβ antibody (Aducanumab) and anti-CD22 antibody (SM03).

### 7.6.7 Example 7: Effects of CD22-AB Bispecific Antibodies on Aβ-Induced IL-1B Release in Human Peripheral Mononuclear Cells (PBMC)

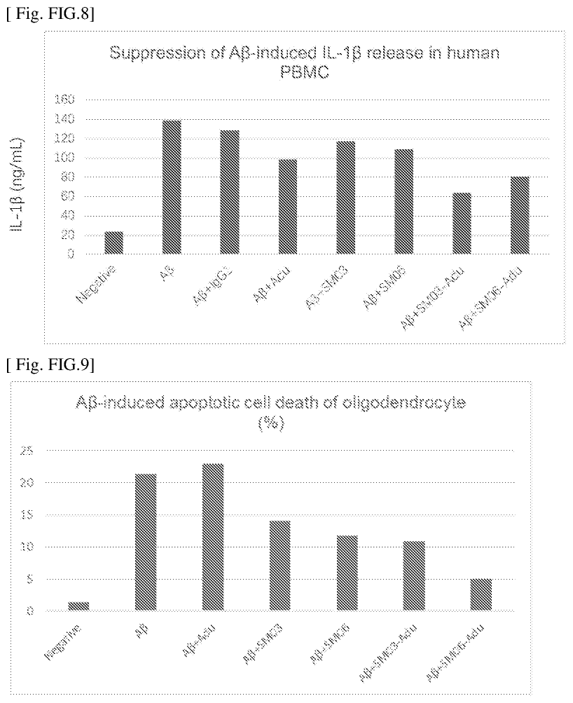

Briefly, human PBMC are seeded onto 96 well plate at a density of 5×10⁵ cell per well. PBMC are incubated with 20 μg/mL Aβ for 2 days. PBMC are co-incubated with antibodies. After 2 days of incubation, supernatant are collected and examined with IL-13 level with ELISA kit (R&D systems). FIG. 8 illustrates a representative result when SM03-Adu and SM06-Adu bispecific antibody are used in the study. It is found that PBMC cells treated with SM03-Adu and SM06-Adu has stronger suppressive effect on IL-1B release in PBMC when compared with anti-Aβa antibody (Aducanumab) and anti-CD22 antibodies (SM03 and SM06). less PSD-95 presented in the cells, suggesting that it reduces phagocytosis of synaptic structure by HMC-3 cells.

### 7.6.8 Example 8: Effect of CD22-Aβ Bispecific Antibody on Aβ-Induced Apoptotic Cell Death on Human Oligodendrocyte

CD22 are expressed in human oligodendrocyte. Briefly, human oligodendrocyte cell line MO3.13 are seeded onto 24 well plate at a density of 0.026 million cells/cm². MO3.13 cells are then activated with 5 μM amyloid β and treated with CD22-AB bispecific antibody for 1 day. Apoptotic cell death of MO3.13 are examined with Annexin V apoptosis kit (Invitrogen). FIG. 9 illustrates a representative result when SM03-Adu and SM06-Adu bispecific antibody are used in the study. It is found that SM03-Adu and SM06-Adu have better suppressive effect on Aβ-induced apoptotic cell death in MO3.13.

### 7.6.9 Example 9: Comparing the Rate of Internalization of CD22, CD33 or CD74-Aβ Bispecific Antibody into Human Microglia Cell Line

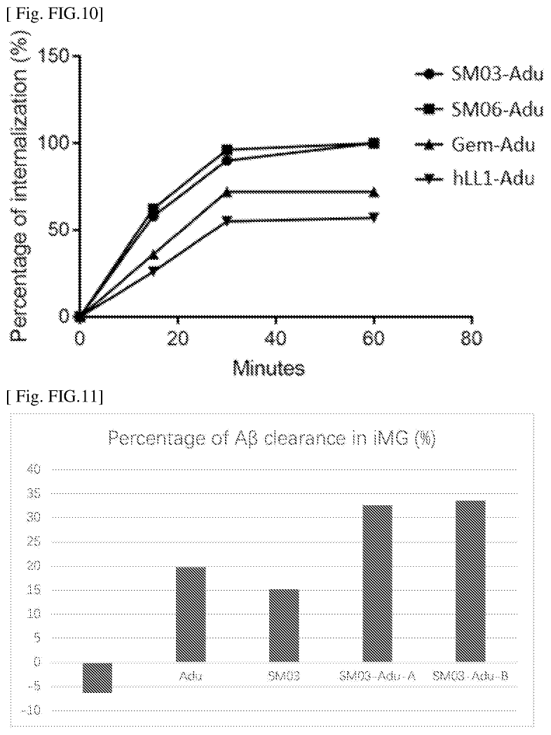

Antibodies that bind to CD22, CD33 or CD74 are internalized in B cells as well as microglia cells. To examine the rate of internalization of these bispecific antibodies into human microglia cells, HMC3 cells are seeded onto 1% gelatin-coated coverslip at a density of 0.026 million cell/cm². Cells are treated with 10 μg/mL CD22, CD33 or CD74-Aβ bispecific antibody and incubate on ice for 1 hour. Then cells are incubated at 37° C. to induce internalization. At designated time points, medium is removed, and cells are fixed with 4% PFA. Surface bispecific antibody are each detected with AlexaFlour 488-conjugated anti Human IgG antibody (Invitrogen). As shown in FIG. 10, it is found that SM03-Adu and hLL1-Adu have the fastest rate of internalization. SM06-Adu demonstrates a slightly slower rate of internalization. The rate of internalization of Gem-Adu bispecific antibody is slower than SM06-Adu and has 50% internalization rate after 30 minutes. Other bispecific antibodies are examined using the above protocol and the result is summarized in Table VI.

TABLE VI

| Antigen | Bispecific Antibody | Rate of internalization |
|---|---|---|
| CD22 | SM03-Adu | ++++ |
| | SM03-BAN | ++++ |
| | SM03-Gan | ++++ |
| | SM03-Cre | ++++ |
| | SM06-Adu | +++ |
| | SM06-BAN | +++ |
| | SM06-Gan | +++ |
| | SM06-Cre | +++ |
| | SM06-AD38 | +++ |
| | hLL2-Adu | ++ |
| | hLL2-Cre | ++ |
| | hLL2-Gan | ++ |
| | hLL2-BAN | ++ |
| CD33 | Gem-Adu | ++ |
| | 96-Adu | ++ |
| | Lin-Adu | ++ |
| CD74 | hLL1-Adu | +++++ |

Note:
“+++++” indicates highest rate while “+” indicates the slowest rate

### 7.6.10 Example 10: Aβ Clearance in Induced Human Microglia by CD22, CD33 or CD74-Aβ Bispecific Antibodies

One of the embodiments of the present invention is to exploit the internalizing characteristics of anti-CD22, anti-CD33 and anti-CD74 moieties of the bispecific antibodies to expedite internalization or phagocytosis of toxic oligomeric Aβ protein captured by the anti-Aβ moieties of the bispecific antibodies. The induction of oligomeric Aβ phagocytosis in induced human microglia cells (iMG) is examined as follows. Briefly, iMG are generated by seeding 1.25 million human peripheral blood mononuclear cells onto 0.02% Geltrex-coated well in 24 well plate. Twenty-four hours after plating, the suspension cells are removed and the adhere monocyte are washed with PBS once. The adhered monocytes are differentiated with 0.1 μg/ml IL-34 and 0.01 μg/ml GM-CSF for 14 days. Fresh differentiation medium are change every 2-3 days. For the study of Aβ clearance by iMG, iMG are incubated with 0.5 μg/mL biotinated Aβ for 30 minutes. Antibodies are co-treated for the same period of time. After 30 minutes of treatment, the supernatant are collected and the amount of biotinated Aβare quantified by ELISA. ELISA stripes are first coated with anti-Aβ antibody (Clone: 6E10) overnight. Coated stripes are wash once with PBS and blocked with 1% BSA in PBS for 2 hours. Stirpes are washed twice with PBS. Supernatant are added and are incubated for 2 hours. Stripes are then wash with PBS for 3 times. Stripes are incubated with Streptavidin-HRP for 1 hour. Stripes are then wash with PBS for 4 times and TMB are added for coloration development. The signal are read by Varioskan LUX plate reader (ThermoScientific). FIG. 11 illustrates that 2 different version of SM03-Adu, bispecific antibodies exhibit enhanced Aβ clearance when compared to anti-Aβ monoclonal antibody (Aducanumab) and SM03. Other bispecific antibodies are examined using the same protocol and the result are summarized in Table VII.

TABLE VII

| Antigen | Bispecific Antibody | Rate of Aβ phagocytosis |
|---|---|---|
| CD22 | SM03-Adu | ++++ |
| | SM03-BAN | ++++ |
| | SM03-Gan | ++++ |
| | SM03-Cre | ++++ |
| | SM06-Adu | +++ |
| | SM06-BAN | +++ |
| | SM06-Gan | +++ |
| | SM06-Cre | +++ |
| | SM06-AD38 | +++ |
| | hLL2-Adu | ++ |
| | hLL2-Cre | ++ |
| | hLL2-Gan | ++ |
| | hLL2-BAN | ++ |
| CD33 | Gem-Adu | ++ |
| | 96-Adu | ++ |
| | Lin-Adu | ++ |
| CD74 | hLL1-Adu | +++++ |

Note:
“+++++” indicates highest rate while “+” indicates the slowest rate

While the invention has been described in detail and with reference to specific embodiments thereof, it will be apparent to one skilled in the art that various changes and modifications can be made therein without departing from the spirit and scope of the invention.

1. A method of promoting removal of beta-amyloid (Aβ) plaque in a subject in need thereof, said method comprising the step of administering to the subject a therapeutically effective amount of a bispecific antibody or antigen-binding fragment thereof that specifically binds on one end to an internalizing antigen expressed on the surface of cells in the neuroimmune system and to a toxic form of Aβ at the other end.

2. The method of claim 1 wherein the binding of the bispecific antibody or antigen-binding fragment to the internalizing antigen expressed on the surface of cells in the neuroimmune system (a) induces internalization of the surface receptors/antigens and/or (b) suppresses microglia, astrocyte, oligodendrocyte, pericyte brain endothelial cell or other neuro-immunological cell activation which promote neuro-inflammation and/or (c) reduces pathogenic synaptic pruning by microglia, astrocyte or other neuro-immunological cell and/or (d) modulate antigen presentation by microglia, astrocyte, endothelial cell, or other neuro-immunological cell to infiltrating T cells.

3. The method of claim 1, wherein the bispecific antibody or antigen-binding fragment binds to a toxic form of Aβ selected from the group consisting of (a) a monomeric form; (b) an oligomeric form; (c) a protofibril; (d) a fibril; or (e) a pyroglutamate-modified form.

4. The method of claim 1, wherein said step of administering to the subject the therapeutically effective amount of said bispecific neuroinflammation in said subject, further wherein the binding of the bispecific antibody or antigen-binding fragment to the internalizing antigen expressed on the surface of cells in the neuroimmune system (a) induces internalization of the surface receptors/antigens and/or (b) suppresses microglia, astrocyte, oligodendrocyte, pericyte brain endothelial cell or other neuro-immunological cell activation which promote neuro-inflammation and/or (c) reduces pathogenic synaptic pruning by microglia, astrocyte or other neuro-immunological cell and/or (d) modulate antigen presentation by microglia, astrocyte, endothelial cell, or other neuro-immunological cell to infiltrating T cells.

5. The method of claim 1, wherein the internalizing antigen is CD22, CD33 or CD74 and the cells in the neuroimmune system are selected from among microglia cells, oligodendrocytes and astrocytes.

6. The methods of claim 1, wherein the subject has been diagnosed with clinical or pre-clinical Alzheimer's disease, prodromal Alzheimer's disease, Down's syndrome, clinical or pre-clinical amyloid angiopathy (CAA), Parkinson's disease, multi-infarct dementia, cerebral amyloid angiopathy, glaucoma, pre-eclampsia, cognitive impairment, memory loss, or a vascular disorder caused by pathogenic Aβ peptide in blood vessels.

7. The method of claim 1, wherein said step of administering to the subject the therapeutically effective amount of said bispecific antibody or antigen-binding fragment thereof further serves to treat an Aβ-related disease or disorder in said subject, wherein said bispecific antibody or antigen-binding fragment thereof binds to CD22, CD33 or CD74 on one end and back to Aβ on the other end, further wherein the bispecific antibody or antigen-binding fragment (a) induces internalization of the surface receptors/antigens and/or (b) suppresses microglia, astrocyte, oligodendrocyte, pericyte brain endothelial cell or other neuro-immunological cell activation which promote neuro-inflammation and/or (c) reduces pathogenic synaptic pruning by microglia, astrocyte or other neuro-immunological cell and/or (d) modulate antigen presentation by microglia, astrocyte, endothelial cell, or other neuro-immunological cell to infiltrating T cells.

8. The method of claim 1, wherein said step of administering to the subject the therapeutically effective amount of said bispecific antibody or antigen-binding fragment thereof further serves to treat a disease or disorder associated with neuroinflammation in said subject, wherein said bispecific antibody or antigen-binding fragment thereof binds to CD22, CD33 or CD74 on one end and to Aβ on the other end, further wherein the antibody or antigen-binding fragment (a) induces internalization of the surface receptors/antigens and/or (b) suppresses microglia, astrocyte, oligodendrocyte, pericyte brain endothelial cell or other neuro-immunological cell activation which promote neuro-inflammation and/or (c) reduces pathogenic synaptic pruning by microglia, astrocyte or other neuro-immunological cell and/or (d) modulate antigen presentation by microglia, astrocyte, endothelial cell, or other neuro-immunological cell to infiltrating T cells.

9. The method of claim 8, wherein the neuroinflammation-related disease or disorder is selected from among clinical or pre-clinical Alzheimer's disease, prodromal Alzheimer's disease, Down's syndrome, clinical or pre-clinical amyloid angiopathy (CAA), Parkinson's disease, multi-infarct dementia, cerebral amyloid angiopathy, glaucoma, pre-eclampsia, cognitive impairment, memory loss, and a vascular disorder caused by pathogenic Aβ peptide in blood vessels.

10. The method of claim 7, wherein the Aβ-related disease or disorder is Alzheimer's disease.

11. The method of claim 1, wherein the bispecific antibody or antigen-binding fragment is an antibody selected from the group consisting of an IgG1 antibody, an IgG2 antibody, an IgG3 antibody, and an IgG4 antibody.

12. (canceled)

13. The method of claim 1, wherein the bispecific antibody or antigen-binding fragment is selected from the group consisting of a Fab, a Fab′, a F(ab′)₂, a Fv, a scFv, a (scFv)₂, a single domain antibody (sdAb), and a heavy chain antibody (HCAb).

14. The method of claim 1, wherein the bispecific antibody or antigen-binding fragment has specificity against CD22 and comprises either:
a. a light chain variable region (VL) comprising a VL CDR1, VL CDR2, and VL CDR3 that have the amino acid sequences of SEQ ID NO:1, SEQ ID NO:2, and SEQ ID NO:3, respectively; and a heavy chain variable region (VH) comprising a VH CDR1, VH CDR2, and VH CDR3 that have the amino acid sequence of SEQ ID NO: 4, SEQ ID NO:5, and SEQ ID NO:6, respectively, or
b. a light chain variable region (VL) comprising a VL CDR1, VL CDR2, and VL CDR3 that have the amino acid sequences of SEQ ID NO: 11, SEQ ID NO:12, and SEQ ID NO:13, respectively; and a heavy chain variable region (VH) comprising a VH CDR1, VH CDR2, and VH CDR3 that have the amino acid sequence of SEQ ID NO: 14, SEQ ID NO: 15, and SEQ ID NO: 16, respectively.

15. The method of claim 14, wherein the VL and VH of the anti-CD22 moiety of the bispecific antibody or antibody fragment have the amino acid sequence of SEQ ID NO: 7 and SEQ ID NO: 8, respectively.

16. The method of claim 15, wherein the anti-CD22 binding moiety of the bispecific antibody or antibody fragment is SM03.

17. The method of claim 14, wherein the VL and VH of the anti-CD22 moiety of the bispecific antibody or antibody fragment have the amino acid sequence of SEQ ID NO: 9 and SEQ ID NO: 10, respectively.

18. The method of claim 17, wherein the anti-CD22 binding moiety of the bispecific antibody or antibody fragment is SM06.

19. The method of claim 14, wherein the VL and VH of the anti-CD22 moiety of the bispecific antibody or antibody fragment have the amino acid sequence of SEQ ID No: 17 and SEQ ID NO: 18, respectively.

20. The method of claim 19, wherein the anti-CD22 binding moiety of the bispecific antibody or antibody fragment is LL2.

21. The method of claim 14, wherein the VL and VH of the anti-CD22 moiety of the bispecific antibody or antibody fragment have the amino acid sequence of SEQ ID No: 19 and SEQ ID NO: 20, respectively.
